(19) United States
(12) Patent Application Publication
(10) Pub. No.: US 2025/0120928 A1
(43) Pub. Date: Apr. 17, 2025

(54) SUBEROYLANILIDE HYDROXAMIC ACID (SAHA) DRUGS, CONJUGATES, AND NANOPARTICLES, AND METHODS OF USE THEREOF

(71) Applicant: University of Maryland, Baltimore, Baltimore, MA (US)

(72) Inventors: Ryan M. Pearson, Baltimore, MD (US); Steven Fletcher, Baltimore, MD (US); Nhu Truong, Allentown, PA (US)

(21) Appl. No.: 18/578,606

(22) PCT Filed: Jul. 14, 2022
(86) PCT No.: PCT/US2022/037135

§ 371 Date: Jan. 11, 2024

Related U.S. Application Data
(60) Provisional application No. 63/221,521, filed on Jul. 14, 2021.

(51) Int. Cl. A61K 31/167; A61K 47/69; C07C 235/74

(52) U.S. Cl. CPC A61K 31/167; A61K 47/6937; C07C 235/74

(57) ABSTRACT

The present disclosure provides polymer-drug conjugates comprising suberoylanilide hydroxamic acid (SAHA), derivatives thereof, and salts thereof. The disclosure further provides nanoparticles comprising the polymer-drug conjugate. The disclosure provides methods of treating a disease or disorder in a subject by administering the conjugate or a plurality of nanoparticles comprising the conjugate to the subject. In some embodiments, the disease or disorder is sepsis, septic shock, hemorrhagic shock, or poly-trauma.

### CROSS-REFERENCE TO RELATED APPLICATIONS

The present application is a national stage entry of PCT International Application No. PCT/US2022/037135 filed on Jul. 14, 2022, which claims priority to U.S. Provisional Application No. 63/221,521, filed Jul. 14, 2021, the entirety of each of which is incorporated herein by reference.

### GOVERNMENT RIGHTS

This invention was made with government support under GM142752, AI095190, and HL007698, awarded by the National Institutes of Health. The Government has certain rights in this invention.

### REFERENCE TO SEQUENCE LISTING SUBMITTED ELECTRONICALLY

This application contains a Sequence Listing which has been submitted electronically in XML file format and is hereby incorporated by reference in its entirety. The XML file, named 115834-5032-WO.xml, was created on Jul. 13, 2022 and is 3.93 kilobytes in size.

### BACKGROUND

Sepsis is a life-threatening multifactorial organ dysfunction caused by a dysregulated host response to infection. In 2017, an estimated 48.9 million incident cases of sepsis were recorded worldwide, and sepsis-related deaths represent one-fifth of all global deaths. Despite the gravity of sepsis and septic shock, poor understanding of its pathophysiology has resulted in stagnant improvements in standard of care where treatment mainly consists of supportive care, including early administration of broad-spectrum antibiotics to eliminate infection, early administration of intravenous fluids to offset vascular leakage and endothelial damage, and resuscitation to limit multiorgan damage and augment pulmonary capacity. Therefore, an urgent need exists to develop more effective therapeutics for the treatment of sepsis.

The inflammatory response is initiated by the recognition of pathogens and damaged tissues by pattern recognition receptors (PRRs) that are expressed on innate immune cells in addition to many types of somatic tissues. Toll-like receptors (TLRs) are a family of PRRs, among others, that recognize specific components of microorganisms called pathogen-associated molecular patterns (PAMPs). Similarly, proteins and cellular products released due to tissue damage are recognized as damage-associated molecular patterns (DAMPs). Systemic overactivation of the innate immune system by PAMPs and DAMPs results in a severe inflammatory response characterized by the excessive release of proinflammatory cytokines including TL-6, TNFα, and IFNγ, called the cytokine storm. Specifically, lipopolysaccharide (LPS), a type of PAMP, can induce a dose dependent production of proinflammatory cytokines, which can be toxic at high concentrations. LPS activates innate immune cells through the PRR TLR4 and subsequent initiation of nuclear factor-kB (NF-kB) signaling. This dysregulated immune response results in the potentiation of tissue damage, cellular and molecular dysfunction, and ultimately multi-organ failure and/or death.

During the progression of septic shock, there is an imbalance of histone acetyl transferase (HAT) and histone deacetylase (HDAC) activity, yet higher HDAC activity supersedes and results in a global cellular hypoacetylation. Histone acetylation is a key epigenetic modification that regulates the expression of genes involved in cellular processes including survival, repair/healing, signaling, and proliferation. Dysregulated inflammatory responses in response to infection are characterized by an acute immune activation phase that involves HATs to promote chromatin accessibility and inflammatory gene transcription. This allows for an excessive production and release of proinflammatory cytokines, termed the cytokine storm, resulting in the potentiation of tissue damage, cellular and molecular dysfunction, multi-organ failure, and death. During this progression that can lead to shock, global cellular hypoacetylation is observed where decreases in acetylation of nuclear and cytoplasmic proteins may be attributed to the upregulation of HDAC enzymes. Normalization of acetylation prevents cell death, decreases inflammation, attenuates activation of pro-apoptotic pathways, and augments pro-survival pathways.

HDAC inhibitors (HDACi) are a class of drugs that aim to regulate the HAT/HDAC imbalance through the inhibition of HDAC enzymes, thus normalizing acetylation profiles. Notably, HDACis can be used to restore the HAT/HDAC imbalance and has shown to significantly improve survival in lethal models of septic shock, hemorrhagic shock, and poly-trauma. Improvement in survival is not due to better resuscitation, but due to enhanced ability of cells to tolerate lethal insults. The reduction in proinflammatory cytokine secretions and the regulation of gene expression serves as an attractive strategy to be targeted therapeutically. Excessive immune activation and immunosuppression are critical competing elements of the dysregulated innate and adaptive immune responses in sepsis. A poly-pharmacological strategy is vital to reduce complex drug regimens by exploiting one therapeutic to target the multiple inflammatory mechanisms underlying sepsis.

### SUMMARY OF THE DISCLOSURE

In one aspect, the present disclosure relates to a compound of Formula (I)

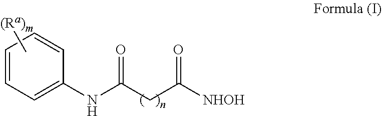

or a pharmaceutically acceptable salt thereof, wherein in Formula (I): m is 0 or 1; n is an integer selected from 1, 2, 3, 4, 5, 6, 7, and 8; and R^{a}, when present, comprises at least one of —OH, C₁-C₃ alkyl, C₁-C₃ alkoxy, —C(O)H, —C(O)OH, —C(O)X; and X is halogen selected from F, Br, Cl, and I. In one embodiment, n is 6. In one embodiment, suberoylanilide hydroxamic acid (SAHA):

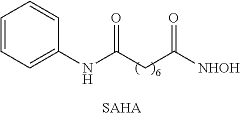

is excluded from Formula (I). In one embodiment, m is 1 and n is 6. In one embodiment, the compound of Formula (I is N¹-hydroxy-N⁸-(4-(hydroxymethyl)phenyl)octanediamide (SAHA-OH):

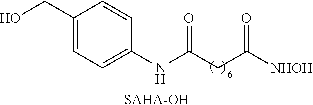

or a pharmaceutically acceptable salt thereof.

In another aspect, the present disclosure provides a conjugate of Formula (10), comprising a polymer moiety (POLY) covalently linked to a drug moiety comprising the compound of Formula (I) or salt thereof:

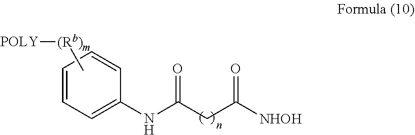

or a pharmaceutically acceptable salt thereof, wherein in Formula (10): m is 0 or 1; n is an integer selected from 1, 2, 3, 4, 5, 6, 7, and 8; and R^{b}, when present, comprises at least one of —O—, —C₁-C₃ alkyl-, —C₁-C₃ alkoxy-, —C(O)—, —C(O)O—. In one embodiment, m is 0 and n is 6. In one the conjugate of Formula (10) is

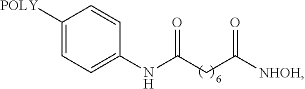

or a pharmaceutically acceptable salt thereof. In one embodiment, m is 1 and n is 6. In one embodiment, the conjugate of Formula (10) is

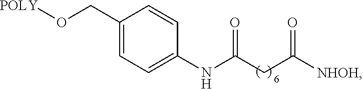

or a pharmaceutically acceptable salt thereof. In one embodiment, the conjugate of Formula (10) is

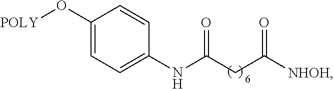

or a pharmaceutically acceptable salt thereof. In one embodiment, POLY is a biodegradable polymer. In one embodiment, POLY is selected from polyglycolic acid (PGA), poly(DL-lactide) (PLA), and poly(D,L-lactide-co-glycolide) (PLGA). In one embodiment, POLY is PLGA. In one embodiment, PLGA comprises lactic acid monomer subunits and glycolic acid monomer subunits in a ratio from about 25:75 to about 75:25 lactic acid monomer subunits:glycolic acid monomer subunits. In one embodiment, PLGA comprises lactic acid monomer subunits and glycolic acid monomer subunits in a ratio of from about 25:75 to about 50:50 lactic acid monomer subunits:glycolic acid monomer subunits. In one embodiment, PLGA comprises lactic acid monomer subunits and glycolic acid monomer subunits in a ratio of from about 50:50 to about 75:25 lactic acid monomer subunits:glycolic acid monomer subunits. In one embodiment, PLGA comprises lactic acid monomer subunits and glycolic acid monomer subunits in a ratio of about 25:75, about 50:50, or about 75:25 lactic acid monomer subunits:glycolic acid monomer subunits. In one embodiment, the PLGA comprises an acid terminus which is covalently linked to the drug moiety to form the conjugate of Formula (10). In one embodiment, POLY is a random copolymer, or has a formula

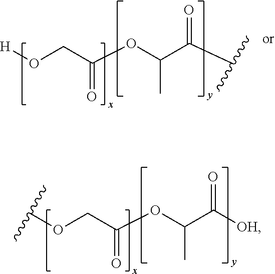

wherein each x and y is independently an integer from 1 to 10,000. In one embodiment, POLY has a number average molecular weight from about 3 kD to about 50 kD.

In yet another aspect, the present disclosure provides a nanoparticle comprising a conjugate of Formula (10) or salt thereof described elsewhere herein. In one embodiment, the nanoparticle further comprises poly(DL-lactide) (PLA). In one embodiment, the PLA is acid-terminated. In one embodiment, the number average molecular weight of PLA is from about 5 kD to about 50 kD. In one embodiment, the nanoparticle further comprises a surfactant. In one embodiment, the surfactant comprises a poloxamer, a poloaxamine, PEG, Tween-80, gelatin, dextran, pluronic L-63, PVA, methylcellulose, lecithin, DMAB, sodium deoxycholate, poly(acrylic acid), hyaluronic acid, vitamin E TPGS (D-α-tocopheryl polyethylene glycol 1000 succinate), or poly(ethylene-alt-maleic anhydride) (PEMA). In one embodiment, the surfactant is PEMA. In one embodiment, the number average molecular weight of PEMA is from about 30 kD to about 500 kD. In one embodiment, the weight average molecular weight of PEMA is about 400 kD. In one embodiment, the conjugate and PLA form a core. In one embodiment, the core is partially or completely coated by poly(ethylene-alt-maleic anhydride) (PEMA). In one embodiment, the loading of the drug moiety in the nanoparticle is from about 0.5 μg/mg to about 70 μg/mg. In one embodiment, the loading of the drug moiety in the nanoparticle is from about 2.5 μg/mg to about 5.0 μg/mg. In one embodiment, the loading of the drug moiety in the nanoparticle is from about 8.0 μg/mg to about 12 μg/mg. In one embodiment, the loading of the drug moiety in the nanoparticle is from about 25 μg/mg to about 35 μg/mg. In one embodiment, the loading of the drug moiety in the nanoparticle is from about 55 μg/mg to about 65 μg/mg. In one embodiment, the average particle size of the plurality of nanoparticles is from about 50 nm to about 800 nm. In one embodiment, the polydispersity index (PDI) of the plurality of nanoparticles is from about 0.05 to about 0.45. In one embodiment, the plurality of nanoparticles have a zeta potential from about 0 mV to about −70 mV.

In yet another aspect, the present disclosure provides a pharmaceutical composition comprising the compound of Formula (I) or salt thereof described elsewhere herein, the conjugate of Formula (10) or salt thereof described elsewhere herein, or the plurality of nanoparticles described elsewhere herein, and a pharmaceutically acceptable carrier.

In yet another aspect, the present disclosure provides a method of treating a disease or disorder in a subject, the method comprising administering to the subject the compound of Formula (I) or salt thereof described elsewhere herein, the conjugate of Formula (10) or salt thereof described elsewhere herein, the plurality of nanoparticles described elsewhere herein, or the pharmaceutical composition described above. In one embodiment, the disease or disorder is an inflammatory disease or disorder or a disease or disorder with a significant inflammatory component. In one embodiment, the disease or disorder is selected from psoriasis, autoimmunity, endometriosis, inflammatory bowel disease, sepsis, septic shock, hemorrhagic shock, and poly-trauma. In one embodiment, the disease or disorder is sepsis. In one embodiment, each of the compound, the conjugate, the plurality of nanoparticles, and the pharmaceutical composition is independently formulated in a solution. In one embodiment, the solution is administered to the subject by injection. In one embodiment, the compound, the conjugate, the plurality of nanoparticles, or the pharmaceutical composition administered to the subject in combination with one or more further therapeutic agents. In one embodiment, the one or more further therapeutic agents are selected from antibiotics, analgesics, steroids, vasopressors, and vasoconstrictors. In one embodiment, the subject is a human subject.

### BRIEF DESCRIPTION OF THE DRAWINGS

FIGS. 1A-1B depict NMR spectra of suberoylanilide hydroxamic acid (SAHA) precursor methyl 8-oxo-8-(phenylamino)octanoate (SF-7-277) measured in CDCl₃-d. FIG. 1A: ¹H-NMR spectrum (500 MHz, CDCl₃-d): δ (ppm) 7.77 (s, 1H), 7.52 (d, J=8.5 Hz, 2H), 7.28 (t, J=7.8 Hz, 2H), 7.07 (t, J=7.3 Hz, 1H), 3.65 (s, 3H), 2.31 (m, J=7.6 Hz, 4H), 1.70 (t, J=7 Hz, 2H), 1.61 (t, J=7.3 Hz, 2H), 1.34 (t, J=3 Hz, 4H). FIG. 1B: ¹³C NMR spectrum (500 MHz, CDCl₃-d): δ (ppm) 174.5, 171.8. 138.3, 129.1, 124.2, 120.0, 77.5, 77.0, 51.7, 37.6, 34.1, 28.9 (2), 25.5, 24.8.

FIGS. 2A-2B depict NMR spectra of SAHA-OH precursor methyl 8-((4-(hydroxymethyl)phenyl)amino)-8-oxooctanoate (SF-7-258) measured in CDCl₃-d. FIG. 2B: ¹H NMR spectrum (400 MHz, CDCl₃-d): δ (ppm) 7.62 (s, 1H), 7.49 (d, J=8.8 Hz, 2H), 7.29 (d, J=7.6 Hz, 2H), 4.64 (s, 2H), 3.67 (s, 3H), 2.36-2.29 (m, 4H), 1.72 (t, J=6 Hz, 2H), 1.63 (t, J=6.8 Hz, 2H). FIG. 2B: ¹³C NMR spectrum (400 MHz, CDCl₃-d): δ (ppm) 174.4, 171.5, 137.4, 136.8, 127.7, 120.0, 64.8, 51.6, 37.5, 33.9, 28.7 (2), 25.3, 24.7.

FIG. 3 is an ¹H-NMR spectrum of SAHA N¹-hydroxy-N⁸-phenyloctanediamide (SF-7-278) measured in DMSO-d₆ (calibrated at 2.5 ppm). ¹H NMR (400 MHz, DMSO-d₆): δ (ppm) 10.30 (s, 1H), 9.81 (s, 1H), 8.63 (s, 1H), 7.56 (d, J=8.8 Hz, 2H), 7.26 (t, J=6.8 Hz, 2H), 6.99 (t, J=6.8 Hz, 1H), 2.27 (t, J=7.2 Hz, 2H), 1.92 (t, J=7.6 Hz, 2H), 1.55 (t, J=6 Hz, 2H), 1.47 (t, J=6.8 Hz, 2H).

FIGS. 4A-4B depict NMR spectra of SAHA-OH N¹-hydroxy-N⁸-(4-(hydroxymethyl)phenyl)octanediamide (SF-7-279) measured in DMSO-d₆ (calibrated at 2.5 ppm). FIG. 4A: ¹H NMR spectrum (400 MHz, DMSO-d₆): δ (ppm) 10.34 (s, 1H), 9.84 (s, 1H), 7.52 (d, J=8 Hz, 2H), 7.19 (d, J=8.8 Hz, 2H), 4.40 (s, 2H), 2.26 (t, J=7.2 Hz, 2H), 1.92 (t, J=6.8 Hz, 2H), 1.55 (t, J=6.4 Hz, 2H), 1.47 (t, J=6.8 Hz, 2H). FIG. 4B: ¹³C NMR spectrum (400 MHz, DMSO-d₆): δ (ppm) 171.5, 169.6, 138.4, 137.5, 127.3, 119.2, 63.0, 40.0 (2 signals obsc by solvent), 36.8, 32.7, 28.9, 25.5.

FIGS. 5A-5B depict the chemical synthesis of SAHA-OH and IC₅₀ values of SAHA and SAHA-OH. FIG. 5A: Reaction schematic showing the introduction of a methine alcohol group at the para position of the phenyl ring to produce SAHA-OH. FIG. 5B: IC₅₀ values of SAHA and SAHA-OH tested against HDAC1, 2, 3, 6, and 8.

FIGS. 6A-6C demonstrate that modification to SAHA maintained similar anti-inflammatory properties. FIGS. 6A-6B: IL-6 (FIG. 6A) and TNFα (FIG. 6B) cytokine measurements of bone marrow macrophages (BMMØs) treated with SAHA or SAHA-OH at various concentrations for 3 hours, then lipopolysaccharide (LPS) treated for 48 hours. FIG. 6C: IC₅₀ curves of unstimulated BMMØs treated with SAHA and SAHA-OH for 48 hours. MTS assay assessed for cellular proliferation. One-way ANOVA and Tukey's multiple comparisons test were performed to determine statistical differences. All groups were compared to each other, and matching letters indicate no statistical differences (p>0.05). All non-matching letters are considered significantly different (p<0.05). Data is expressed as means±SD (n=3) and 3 independent experiments.

FIG. 7 is a heat map depicting the differential cytokine secretion profiles of SAHA and SAHA-OH treated BMMØs under LPS stimulation. Data is plotted as Log 2 fold change relative to LPS.

FIGS. 8A-8F depict the cytokine secretion profile of SAHA and SAHA-OH. IL-6 (FIG. 8A), IFNβ (FIG. 8B), TNFα (FIG. 8C), IL-1β (FIG. 8D), GROα (FIG. 8E), and IL-1β (FIG. 8F) measurements of BMMØs pre-treated with SAHA (10 μM) or SAHA-OH (10 μM) for 3 hours prior to 48-hour LPS stimulation, obtained via multiplex Luminex assay. One-way ANOVA and Tukey's multiple comparisons test were performed to determine statistical differences. All groups were compared to each other, and matching letters indicate no statistical differences (p>0.05). All non-matching letters are considered significantly different (p<0.05). Data is expressed as means±SD (n=3) and 3 independent experiments. NT=no treatment.

FIG. 9 is a chart of MCP-1 cytokine measurement of BMMØs treated with SAHA (10 μM) or SAHA-OH (10 μM). SAHA or SAHA-OH were pre-treated for 3 hours and subjected to LPS treatment for 48 hours. One-way ANOVA and Tukey's multiple comparisons test were performed to determine statistical differences. All groups were compared to each other, and matching letters indicate no statistical differences (p>0.05). All non-matching letters are considered significantly different (p<0.05). All data is expressed as mean±SD (n=3) and 3 independent experiments. NT=no treatment.

FIG. 10 is a Western blot analysis using antibodies against acetyl-histone H3 and acetyl-α-tubulin in unstimulated BMMØs cultured for 4 or 9 hours with 10 μM SAHA or 10 μM SAHA-OH which demonstrates that SAHA-OH retained its ability to acetylate histone H3 and α-tubulin. Histone H3, α-tubulin, and 3-actin was used as loading controls. Data is representative of 3 independent experiments. NT=no treatment.

FIGS. 11A-11E demonstrate that SAHA-OH improved cellular viability and reduced macrophage apoptosis. Unstimulated BMMØs or LPS-challenged BMMØs were pre-treated with varying concentrations of SAHA or SAHA-OH and stained with FITC Annexin V (AV) and propidium iodide (PI). FIGS. 11A and 11C: Flow cytometry analysis defined the percentage of viability (AV−/PI−). LPS-challenged BMMØs were pre-treated with 10 μM SAHA or 10 μM SAHA-OH (FIG. 11C). FIGS. 11B and 11D: Flow cytometry analysis defined the percentage of apoptosis (AV+/PI−). LPS-challenged BMMØs were pre-treated with 10 μM SAHA or 10 μM SAHA-OH (FIG. 11D). FIG. 11E: Representative flow cytometry plots. One-way ANOVA and Tukey's multiple comparisons test were performed to determine statistical differences. All groups were compared to each other, and matching letters indicate no statistical differences (p>0.05). All non-matching letters are considered significantly different (p<0.05). Data is expressed as means±SD (n=3) and 3 independent experiments. NT=no treatment.

FIGS. 12A-12G demonstrate that SAHA-OH improved toxicity profile in B cells isolated from splenocytes. Unstimulated or LPS-challenged splenocytes were pre-treated with varying concentrations of SAHA or SAHA-OH and stained with anti-B220-PE-Cy7 and FITC-Annexin V (AV) and propidium iodide (PI). FIGS. 12A and 12D: Flow cytometry analysis defined the percentage of viability (AV−/PI−). Splenocytes were pre-treated with 10 μM SAHA or SAHA-OH, with or without LPS stimulation (FIG. 12D). FIGS. 12B and 12E: Flow cytometry analysis defined the percentage of apoptosis (AV+/PI−). Splenocytes were pre-treated with 10 μM SAHA or SAHA-OH, with or without LPS stimulation (FIG. 12E). FIGS. 12C and 12F: Flow cytometry analysis defined the percentage of dead cells (AV+/PI+). Splenocytes were pre-treated with 10 μM SAHA or SAHA-OH, with or without LPS stimulation (FIG. 12F). FIG. 12G: Representative flow cytometry plots of B cells. One-way ANOVA and Tukey's multiple comparisons test were performed to determine statistical differences. All groups were compared to each other, and matching letters indicate no statistical differences (p>0.05). All non-matching letters are considered significantly different (p<0.05). Data is expressed as means±SD (n=3) and 3 independent experiments.

FIGS. 13A-13C demonstrate the toxicity profile of SAHA-OH at 30 μM. Unstimulated BMMØ or LPS-challenged BMMØ were pre-treated with 30 μM of SAHA or 30 μM of SAHA-OH and stained with FITC-annexin V (AV) and propidium iodide (PI). FIG. 13A: Flow cytometry analysis defined the percentage of viability (AV−/PI−). FIG. 13B: Flow cytometry analysis defined the percentage of apoptosis (AV+/PI−). FIG. 13C: Representative flow cytometry plots of AV/PI staining on BMMØ. One-way ANOVA and Tukey's multiple comparisons test were performed to determine statistical differences. All groups were compared to each other, and matching letters indicate no statistical differences (p>0.05). All non-matching letters are considered significantly different (p<0.05). All data is expressed as mean±SD (n=3) and 3 independent experiments. NT=no treatment.

FIGS. 14A-14C demonstrate that SAHA-OH reduced SAHA-associated splenic damage and maintained similar suppression of proinflammatory cytokine secretions in mouse plasma. FIG. 14A: In vivo i.p. dosing regimen of a prophylactic LPS-induced endotoxemia C57BL/6 male mice model. FIGS. 14B-14C: IL-6 (FIG. 14B) and TNFα (FIG. 14C) cytokine measurements on mouse plasma. FIG. 14D: Representative hematoxylin and eosin (H&E) stains of spleen and liver slices extracted from the same cohort of mice. Scale bars for spleen slices are 520 μm. Scale bars for liver slices are 100 μm. One-way ANOVA and Tukey's multiple comparisons test were performed to determine statistical differences. All groups were compared to each other, and matching letters indicate no statistical differences (p>0.05). All non-matching letters are considered significantly different (p<0.05). Data is expressed as means±SD (n=6 mice per group). NT=no treatment.

FIG. 15 is a graphical schematic of the formulation and proposed multimodal mechanism of action of immunomodulatory nanoparticle (iNP)-SAHA via metabolic and epigenetic reprogramming of inflammatory responses. (A) SAHA was modified to SAHA-OH and (B) precisely conjugated with poly(lactic-co-glycolic acid) (PLGA) to form a PLGA-SAHA prodrug. (C) PLGA-SAHA was used to form iNP-SAHA nanoparticles at precise nanoformulations. (D) The treatment of macrophages with iNP-SAHA led to its internalization through endocytosis, where endosomal degradation led to SAHA-OH and lactic acid as cleavage products. Both components play a role in metabolic and epigenetic reprogramming that occurs within the nucleus. Lactic acid and SAHA-OH has demonstrated inhibitory effects on the NF-KB P38 MAPK signaling pathway that ultimately resulted in inhibited inflammatory responses. SAHA-OH inhibits HDACs, thus allowing HATs to transcriptionally activate gene expression for a pro-survival response.

FIGS. 16A-16B depict the synthesis and characterization of PLGA-SAHA conjugates. FIG. 16A: EDC/NHS reaction of PLGA and SAHA-OH to synthesize PLGA-SAHA conjugates. FIG. 16B: ¹H-NMR spectrum of PLGA, SAHA-OH, and PLGA-SAHA measured in DMSO-d₆ (calibrated at 2.5 ppm). Coupling efficiency was measured to be 77.5%. ¹H NMR (400 MHz, DMSO-d₆) of PLGA: δ (ppm) 5.20 (m, 1H), 4.91 (s, 2H), 1.47 (m, 3H). ¹H NMR (400 MHz, DMSO-d₆) of SAHA-OH: δ (ppm) 7.52 (d, J=8 Hz, 2H), 7.19 (d, J=8.8 Hz, 2H), 4.40 (s, 2H), 2.26 (t, J=7.2 Hz, 2H), 1.92 (t, J=6.8 Hz, 2H), 1.55 (t, J=6.4 Hz, 2H), 1.47 (t, J=6.8 Hz, 2H). ¹H NMR (400 MHz, DMSO-d₆) of PLGA-SAHA: δ (ppm) 7.52 (d, J=8 Hz, 2H), 7.19 (d, J=8.8 Hz, 2H), 5.20 (m, 1H), 4.91 (s, 2H), 4.40 (s, 2H), 2.26 (t, J=7.2 Hz, 2H), 1.92 (t, J=6.8 Hz, 2H), 1.55 (t, J=6.4 Hz, 2H), 1.47 (m, 2H).

FIGS. 17A-17B depict the synthesis of iNPs. FIG. 17A: Schematic of the precise nanoformulation of PLGA-SAHA prodrug and unmodified PLA. FIG. 17B: Specific stoichiometric ratios to synthesize two loadings of iNP-SAHA via o/w single emulsion technique.

FIG. 18 demonstrates that iNPs do not acetylate histone H3 or α-Tubulin. BMMØs were pre-treated for 3 hours with cargo-less iNPs at 300 μg/mL. Excess iNPs were washed with PBS and allowed to incubate for either 1, 6, or 24 hours (4 hours, 9 hours, and 27 hours, respectively). Cell lysates were collected, and Western blot analysis used antibodies against acetylated histone H3 and acetylated α-tubulin. Histone H3 and α-Tubulin was used as loading controls.

FIG. 19 demonstrates that iNP-SAHA_{High} effectively acetylates histone H3 and α-Tubulin. BMMØs were pre-treated for 3 hours with iNP-SAHA_{Low} or iNP-SAHA_{High} at 300 μg/mL. Excess iNPs were washed with PBS and allowed to incubate for either 6 or 45 hours (9 and 48 hours total, respectively). Cell lysates were collected, and western blot analysis used antibodies against acetylated histone H3 and acetylated α-tubulin. Histone H3, α-Tubulin, and β-Actin was used as loading controls.

FIG. 20 demonstrates that iNP-SAHA delivers SAHA-OH to BMMØs and acetylates histone H3. The immunocytochemistry (ICC) of BMMØs stained for DAPI and acetylated histone H3 was obtained. BMMØs were cultured for 3 hours with iNP-SAHA_{Low}-Cy5.5, and iNP-SAHA_{High}-Cy5.5, excess NPs were washed off, and imaged 48 hours later via confocal microscopy. Scale bars are 10 μm.

FIG. 21 depicts that iNP-SAHA is internalized in BMMØs. The immunocytochemistry (ICC) of BMMØs stained for DAPI and acetylated histone H3 was obtained. BMMØs were cultured for 3 hours with iNP-SAHA_{Low}-Cy5.5, excess NPs were washed off, and imaged 48 hours later via confocal microscopy. Z-stacked images demonstrate internalization of iNP-SAHA_{Low} within the cell.

FIG. 22 demonstrates that the uptake of iNP-SAHA in BMMØs behaved similarly under inflammatory-like conditions. The immunocytochemistry (ICC) of BMMØs stained for DAPI and acetylated histone H3 was obtained. BMMØs were cultured for 3 hours with iNP-SAHA_{Low}-Cy5.5, excess NPs were washed off, and imaged 48 hours later via confocal microscopy. To simulate an inflammatory state, BMMØs were cultured for 3 hours with iNP-SAHA_{Low}-Cy5.5, excess NPs were washed off, subjected to 300 ng/mL LPS stimulation, and imaged 48 hours later via confocal microscopy. Scale bars are 10 μm.

FIGS. 23A-23H demonstrate that iNP-SAHA can suppress pro-inflammatory cytokine secretions and apoptosis and modify gene expression profiles in vitro. FIG. 23A: Heat map depicting the differential cytokine secretion profiles of BMMØs treated with iNPs (300 μg/mL), iNP-SAHA_{Low} (300 μg/mL), or iNP-SAHA_{High} (300 μg/mL) under LPS stimulation. FIG. 23B: IL-6 measurements of BMMØs treated with SAHA (10 μM), SAHA-OH (10 μM), iNPs, iNP-SAHA_{Low}, or iNP-SAHA_{High}. SAHA or SAHA-OH were pre-treated for 3 hours and subjected to LPS treatment for 48 hours. iNP, iNP-SAHA_{Low}, and iNP-SAHA_{High} were incubated for 3 hours, excess NPs were washed with PBS, and subjected to LPS treatment for 48 hours. FIG. 23C: Flow cytometry analysis defined the percentage of live (AV−/PI−), apoptosis (AV+/PI−), and dead (AV+/PI+) BMMØs cells treated with iNP, iNP-SAHA_{Low}, or iNP-SAHA_{High}. FIG. 23D: Representative flow cytometry plots of AV/PI staining. FIGS. 23E-23H: Nanostring analysis of 248 inflammatory genes, plotted as volcano plots comparing LPS to NT (FIG. 23E), iNP (+LPS) to LPS (FIG. 23F), iNP-SAHA (+LPS) to LPS (FIG. 23G), and iNP-SAHA to iNP (+LPS) (FIG. 23H). BMMØs were treated with iNP or iNP-SAHA_{High} and LPS stimulated for 48 hours. All two gradient colored heat maps represents log 2 fold change relative to LPS control. One-way ANOVA and Tukey's multiple comparisons test was performed to determine statistical differences. All groups were compared to each other, and matching letters indicate no statistical differences (p>0.05). All non-matching letters are considered significantly different (p<0.05). All data is expressed as means±SD (n=3). NT=no treatment.

FIGS. 24A-24F are charts of cytokine secretion by BMMØs. FIG. 24A: GROα measurements of BMMØs treated with SAHA (10 μM), SAHA-OH (10 μM), iNPs (300 μg/mL), iNP-SAHA_{Low}(300 μg/mL), or iNP-SAHA_{High} (300 μg/mL). FIG. 24B: IL-10 measurements of BMMØs treated with SAHA (10 μM), SAHA-OH (10 μM), iNPs (300 μg/mL), iNP-SAHA_{L}ow (300 μg/mL), or iNP-SAHA_{High} (300 μg/mL). FIG. 24C: IL-10 measurements of BMMØs treated with SAHA (10 μM), SAHA-OH (10 μM), iNPs (300 μg/mL), iNP-SAHA_{Low} (300 μg/mL), or iNP-SAHA_{High} (300 μg/mL). FIG. 24D: MCP-1 measurements of BMMØs treated with SAHA (10 μM), SAHA-OH (10 μM), iNPs (300 μg/mL), iNP-SAHA_{Low} (300 μg/mL), or iNP-SAHA_{High} (300 μg/mL). FIG. 24E: TNFα measurements of BMMØs treated with SAHA (10 μM), SAHA-OH (10 μM), iNPs (300 μg/mL), iNP-SAHA_{L}ow (300 μg/mL), or iNP-SAHA_{High} (300 μg/mL). FIG. 24F: IFNβ measurements of BMMØs treated with SAHA (10 μM), SAHA-OH (10 μM), iNPs (300 μg/mL), iNP-SAHA_{Low} (300 μg/mL), or iNP-SAHA_{High} (300 μg/mL). SAHA or SAHA-OH were pre-treated for 3 hours and subjected to LPS treatment for 48 hours. iNP, iNP-SAHA_{Low}, and iNP-SAHA_{High} were incubated for 3 hours, excess NPs were washed with PBS, and subjected to LPS treatment for 48 hours.

FIGS. 25A-25E depict biodistribution and survival rate of prophylactic LPS-induced endotoxemia C57BL/6 male mice. FIG. 25A: Mice were pre-treated with either saline, soluble PLGA-Cy5.5 conjugates (Cy5.5), or iNP containing Cy5.5 (iNP-Cy5.5) for 3 hours. Mice were then subjected to either saline or 20 mg/kg LPS challenge for 3 hours. Spleen, left and right kidneys, liver, heart, lung, and GI tract were isolated and analyzed via in vivo imaging systems (IVIS). FIG. 25B: Average fluorescence intensity (FL) was quantified from the region of interest (ROI). No fluorescence intensity of hearts was detectable. Bars represent an average of 3 mice per treatment group. FIG. 25C: Dose escalation studies of i.p. administered 2 mg iNP-SAHA_{Med} (n=15), 1 mg iNP-SAHA_{Med} (n=10), or 0.5 mg iNP-SAHA_{Med} (n=10) 3 hours prior to 30 mg/kg LPS challenge. FIG. 25D: Dose escalation studies of i.p. administered 2 mg iNP (n=15), 1 mg iNP (n=5), and 0.5 mg iNP (n=5) 3 hours prior to 30 mg/kg LPS challenge. FIG. 25E: Mice were pre-treated with i.p. administered 2 mg/mouse iNP-SAHA (n=15) or 2 mg/mouse iNP (n=15) 3 hours prior to 30 mg/kg LPS treatment. Control LPS mice (n=15) subjected to only 30 mg/kg LPS treatment and pre-treated with saline (20 μL/g body weight). Mice were monitored for 7 days to assess survival. Kaplan-Meier curves and log-rank (Mantel-Cox) tests were performed to compare the survival rates to each survival group. *p<0.05, **p<0.01, ***p<0.001, and ****p<0.0001.

FIG. 26 is an LPS mortality study analysis to establish lethality. Dose escalation studies of i.p. administered 20 mg/kg, 30 mg/kg, or 40 mg/kg LPS (n=5 each) were obtained. Kaplan-Meier curves and log-rank (Mantel-Cox) tests were performed to compare the survival rates to each survival group.

FIGS. 27A-27D demonstrate that iNP-SAHA reduces plasma levels of proinflammatory cytokines and is biocompatible in mouse organ systems. FIG. 27A: In vivo i.p. dosing regimen of a prophylactic LPS-induced endotoxemia C57BL/6 male mice model. LPS control group (n=4) was pretreated with saline (20 μL/g body weight) for 3 hours and subjected to 20 mg/kg LPS i.p. injections. FIG. 27B: Multiplex Luminex analysis of 26 cytokines and chemokines were performed on mouse plasma. Log 2 fold change relative to LPS is represented in a two gradient color heat map. FIG. 27C: Differential suppression of IL-18, GM-CSF, TNFα, MCP-1, MIP-1α, RANTES, TL-4, GROα, and MIP-10 were plotted to compare LPS, iNP, and iNP-SAHA treated mice. FIG. 27D: H&E stains of spleen and liver slices extracted from iNP or iNP-SAHA treated mice. Scale bars for spleen slices are 520 μm. Scale bars for liver slices are 50 μm. One-way ANOVA and Tukey's multiple comparisons test was performed to determine statistical differences. *p<0.05. All data is expressed as mean±SD (n=4 or 5).

FIGS. 28A-28B depict the synthesis and preparation of immunomodulatory SAHA nanoparticles (iNP-SAHA) and the release of SAHA from iNP-SAHA. FIG. 28A: Three different loadings of iNP-SAHAs were prepared by mixing the PLGA-SAHA conjugate with unmodified PLA polymer at precise stoichiometric ratios via single oil-in-water (o/w) emulsion technique. FIG. 28B: The release of SAHA from iNP-SAHA_{Med} was measured over 96 hours in phosphate buffered saline (PBS) and acetate buffer (AB).

FIG. 29 is a chart of iNP-SAHA loading and physiochemical characterization, as measured using DLS.

FIGS. 30A-30D: demonstrate the anti-inflammatory properties of iNP-SAHA through the suppression of pro-inflammatory cytokine mediators along with improving cellular viability upon LPS challenge. FIGS. 30A-30B: iNP-SAHA formulations performed significantly better than cargo-less iNPs at suppressing proinflammatory cytokine secretions from BMMØs stimulated with LPS. FIGS. 30C-30D: iNP-SAHA_{Med} significantly improved viability and almost completely reversed apoptosis and necrosis in BMMØs treated with LPS.

FIGS. 31A-31C demonstrate the concentration-dependent effects of iNP-SAHA treatment on the reduction of pro-inflammatory cytokine secretions. The data show that BMMØs treated with iNP-SAHA at 300 μg/mL LPS was most effective.

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include, but are not limited to, the compounds described herein and, more specifically, compounds of any of formula (I), formula (10), formula (11), formula (12), formulas 1001-1240, and their features and limitations as described herein.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., increased sensitivity to apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs disclosed herein, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule.

As used herein, the term “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of a compound of any of formula (I), formula (10), formula (11), formula (12), formulas 1001-1240. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone.

Additional types of prodrugs are also encompassed. For instance, free carboxyl groups can be derivatized as amides or alkyl esters (e.g., methyl esters and acetoxy methyl esters).

Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of any of formula (I), formula (10), formula (11), formula (12), formulas 1001-1240) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H.

Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)R^{a} (where t is 1 or 2), —S(O)R^{a} (where t is 1 or 2), —S(O)ₜOR^{a} (where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O) OR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)—and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)₂—N(R^{a})—C(═O)—radical, where R^{a} is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)R^{a} (where t is 1 or 2), —S(O) R^{a} (where t is 1 or 2), —S(O)ₜOR^{a} (where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)R^{a} (where t is 1 or 2), —S(O)ₜOR^{a}(where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O) OR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O) OR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” or “Het” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10α-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)R^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)—(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus, such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the disclosure. All embodiments of the disclosure can, in the alternative, be more specifically defined by any of the transitional terms “comprising,” “consisting essentially of,” and “consisting of”

### Compounds

In one embodiment, the present disclosure relates to a compound of Formula (I):

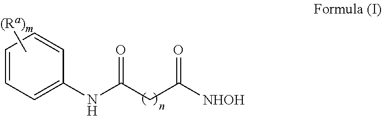

or a pharmaceutically acceptable salt thereof, wherein in Formula (I):
m is 0 or 1;
n is an integer selected from 1, 2, 3, 4, 5, 6, 7, and 8;
R^{a}, when present, comprises at least one of —OH, —C(O)H, —C(O)OH, —C(O)X, —NR¹₂, —SR¹, —S(O)OH, —S(O)R¹, —S(O)₂OH, —CR¹₃—, —C(O)SR¹, —SC(O)OH, —SC(O)X, —C(O)NR¹₂, —NR¹C(O)OH, —NR¹C(O)X, —C(O)NR¹S(O)₂OH, —SO₂NR¹C(O)OH, —SO₂NR¹C(O)X, —OC(O)OH, —OC(O)SR¹, —SC(O)OH, —OC(O)NR¹₂, —NR¹C(O)OH, —S(O)ₜN(R¹)₂ (where t is 1 or 2), —N(R¹)₂S(O)ₜOH (where t is 1 or 2), optionally substituted heteroalkyl, optionally substituted alkyl, optionally substituted alkoxy, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, and optionally substituted heteroarylalkyl;
wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR¹, —SR¹, —OC(O)—R¹, —SC(O)—R¹, —N(R¹)₂, —C(O)R¹, —C(O)OR¹, —C(O)SR¹, —OC(O)N(R¹)₂, —C(O)N(R¹)₂, —N(R¹)C(O)OR¹, —N(R¹)C(O)R¹, —N(R¹)C(O)N(R¹)₂, —N(R¹)C(NR¹)N(R¹)₂, —N(R¹)S(O)ₜR¹ (where t is 1 or 2), —S(O)ₜR¹ (where t is 1 or 2), —S(O) OR¹ (where t is 1 or 2), —S(O)N(R¹)₂ (where t is 1 or 2), and PO₃(R¹)₂; and
R¹ is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
X is halogen selected from F, Br, Cl, and I.

In one embodiment, the compound of Formula (I) is a drug moiety.

In one embodiment, R^{a}, when present, comprises at least one of —OH, —C(O)H, —C(O)OH, —C(O)X, optionally substituted alkyl, and optionally substituted alkoxy. In one embodiment, R^{a}, when present, comprises at least one of —OH, —C(O)H, —C(O)OH, —C(O)X, C₁-C₃ alkyl, and C₁-C₃ alkoxy. In one embodiment, R^{a}, when present, is selected from —OH, —C(O)H, —C(O)OH, —C(O)X, C₁-C₃ alkyl, C₁-C₃ alkoxy, and combinations thereof.

In one embodiment, m is 0 and n is 6. In one embodiment, the compound of Formula (I) is suberoylanilide hydroxamic acid (SAHA) having the following structure:

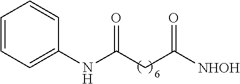

or a pharmaceutically acceptable salt thereof.

In another embodiment, m is 1 and n is 6. In one embodiment, R^{a} is —CH₂OH. In one embodiment, the compound of Formula (I) is N¹-hydroxy-N⁸-(4-(hydroxymethyl)phenyl)octanediamide (SAHA-OH) having the following structure:

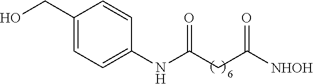

or a pharmaceutically acceptable salt thereof.

In one embodiment, the compound of Formula (I) is a histone deacetylase HDAC inhibitor. In one embodiment, the compound inhibits at least one of HDAC1, HDAC2, HDAC3, HDAC6, and HDAC8. In one embodiment, the compound is a pan-HDAC inhibitor and non-specifically inhibits HDAC enzymes. In another embodiment, the compound is a selective HDAC inhibitor. In one embodiment, the compound selectively inhibits one HDAC enzyme. In one embodiment, the drug moiety selectively inhibits HDAC6.

### Conjugates

In another aspect, the present disclosure provides a conjugate comprising a drug moiety of Formula (I) covalently linked to a polymer. In one embodiment, R^{a} of Formula (I) is present (m=1) and R^{a} reacts with a terminus of the polymer, forming a covalent bond between the drug moiety and the polymer. In one embodiment, R^{a} or a portion of R^{a} acts as a leaving group, providing an open valence for the polymer to bond. In one embodiment, a portion of the polymer acts as a leaving group, providing an open valence for the drug moiety to bond. In one embodiment, the polymer is a biodegradable polymer.

In one embodiment, a polymer moiety (POLY) is covalently linked to a drug moiety comprising a compound of Formula (I), to form a conjugate of Formula (10):

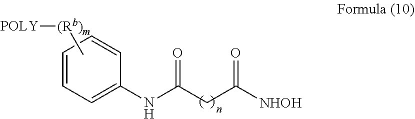

or a pharmaceutically acceptable salt thereof, wherein in Formula (10):
m is 0 or 1;
n is an integer selected from 1, 2, 3, 4, 5, 6, 7, and 8; and
R^{b}, when present, comprises at least one —NR²—, —S—, —S(O)—, —S(O)₂—, —O—, —C(R²)₂—, —C(O)O—, —OC(O)—, —C(O)S—, —SC(O)—, —C(O)NR²—, —NR²C(O)—, —C(O)NR²SO₂—, —SO₂NR₂C(O)—, —OC(O)O—, —OC(O)S—, —SC(O)O—, —OC(O)NR²—, —NR²C(O)O—, —S(O)ᵤN(R²)—(where u is 1 or 2), —N(R²)S(O)ᵤ— (where u is 1 or 2), disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;
wherein any optional substituent is independently selected at each occurrence from optionally substituted alkyl, optionally substituted heteroalkyl, optionally substituted alkenyl, optionally substituted alkynyl, optionally substituted cycloalkyl, optionally substituted heterocycloalkyl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR², —SR², —OC(O)—R², —SC(O)—R², —N(R²)₂, —C(O)R², —C(O)OR², —C(O)SR², —OC(O)N(R²)₂, —C(O)N(R²)₂, —N(R²)C(O)OR², —N(R²)C(O)R², —N(R²)C(O)N(R²)₂, —N(R²)C(NR²)N(R²)₂, —N(R²)S(O)ᵤR² (where u is 1 or 2), —S(O)ᵤR² (where u is 1 or 2), —S(O)ᵤOR² (where u is 1 or 2), —S(O)ᵤN(R²)₂ (where u is 1 or 2), and PO₃(R²)₂; and
R² is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl.

In one embodiment, POLY is a biodegradable polymer.

In one embodiment, R^{a} of Formula (I) forms R^{b} of Formula (10) following a reaction between the drug moiety and the biodegradable polymer. In one embodiment, a portion of R^{a} acts as a leaving group during a reaction between the drug moiety and the biodegradable polymer, providing an open valence on R^{b} which forms the covalent bond to POLY.

In one embodiment, R^{b}, when present, comprises at least one —O—, disubstituted alkyl, disubstituted alkoxy, —C(O)—, and —C(O)O—. In one embodiment, R^{b}, when present, is selected from —O—, —C₁-C₃ alkyl-, —C₁-C₃ alkoxy-, —C(O)—, —C(O)O—, and combinations thereof.

In one embodiment, m is 0 and n is 6. In one embodiment, the conjugate of Formula (10) is:

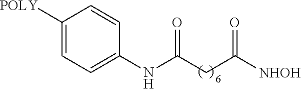

or a pharmaceutically acceptable salt thereof.

In another embodiment, m is 1 and n is 6. In one embodiment, R^{b} is —O—. In one embodiment, the conjugate of Formula (10) is:

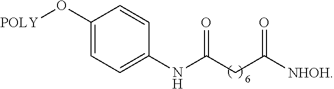

In one embodiment, R^{b} is —OCH₂—. In one embodiment, the conjugate of Formula (10) is:

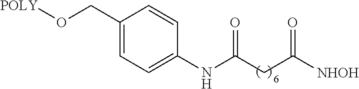

or a pharmaceutically acceptable salt thereof.

POLY can be any polymer known to a person of skill in the art. In one embodiment, POLY comprises polyesters of hydroxy carboxylic acids, polyanhydrides of dicarboxylic acids, or copolymers of hydroxy carboxylic acids and dicarboxylic acids. More generally, POLY may comprise polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Polymers comprising ester and anhydride bonds (e.g., copolymers of glycolic and sebacic acid) may also be employed. For example, POLY may comprise polystyrene, polyglycolic acid polymers (PGA), polylactic acid polymers (PLA), polysebacic acid polymers (PSA), polylactic-co-glycolic) acid copolymers (PLGA or PLG; the terms are interchangeable), [rho]oly(lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers comprise copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, POLY may comprise biologically important amino acids with reactive side chain groups, such as lysine, arginine, aspartic acid, glutamic acid, serine, threonine, tyrosine and cysteine, or their enantiomers, may be included in copolymers with any of the above. POLY may also comprise non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons.

In one embodiment, POLY comprises PLA. In one embodiment, POLY is PLA or a salt thereof. In one embodiment, POLY comprises PGA. In one embodiment, POLY is PGA or a salt thereof.

In one embodiment, POLY is poly(D,L-lactide-co-glycolide) (PLGA) or a salt thereof. In one embodiment, PLGA is a random copolymer of lactic acid monomer subunits and glycolic acid monomer subunits. In another embodiment, PLGA is a block copolymer of lactic acid monomer subunits and glycolic acid monomer subunits. In one embodiment, the ratio of lactic acid monomer subunits to glycolic acid monomer subunits in the PLGA is about 5:95, about 15:85, about 25:75, about 35:65, about 45:55, about 50:50, about 55:45, about 65:35, about 75:25, about 85:15, or about 95:5. In one embodiment, the PLGA comprises an acid terminus. In one embodiment, the acid terminus is covalently linked to the drug moiety to form the conjugate of Formula (10), with —OH leaving as H₂O. In one embodiment, POLY has a formula:

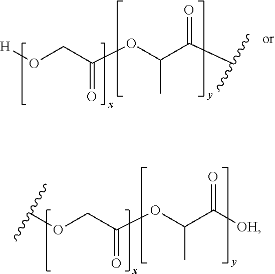

or a salt of either thereof, wherein each x and y is independently an integer from 1 to 10,000. In one embodiment, POLY has a number average molecular weight from about 0.1 kD to about 100 kD, about 0.1 kD to about 90 kD, about 0.1 kD to about 80 kD, about 0.1 kD to about 70 kD, about 0.1 kD to about 60 kD, about 0.1 kD to about 50 kD, about 0.1 kD to about 45 kD, about 0.1 kD to about 40 kD, about 0.1 kD to about 35 kD, about 0.1 kD to about 30 kD, about 0.1 kD to about 25 kD, about 0.1 kD to about 20 kD, about 0.1 kD to about 15 kD, about 0.1 kD to about 10 kD, about 0.5 kD to about 10 kD, about 0.5 kD to about 5 kD, or about 3 kD to about 5 kD.

### Particles

In another aspect, the present disclosure provides a particle comprising a compound of Formula (I), a conjugate of Formula (10), or a pharmaceutically acceptable salt of either one thereof. In one embodiment, the particle is a nanoparticle, a glass particle, a silica particle, a diamond particle, a lipid particle, or a quantum dot. In one embodiment, the surface of the particle is modified with a compound of Formula (I) or a conjugate of Formula (10). In one embodiment, a compound of Formula (I) or a conjugate of Formula (10) is covalently bonded to the surface of the particle. In another embodiment, a compound of Formula (I) or a conjugate of Formula (10) is electrostatically or ionically associated with the surface of the particle. In another embodiment, a compound of Formula (I) or a conjugate of Formula (10) is non-covalently encapsulated within the particle using a suitable method in the art (e.g., double emulsion). In another embodiment, a compound of Formula (I) or a conjugate of Formula (10) is incorporated in a lipid or modifies the surface of a lipid, wherein the lipid is then contained in a particle such as a nanoparticle.

In one embodiment, the particle is a nanoparticle. In one embodiment, the nanoparticle is a therapeutic nanoparticle. In one embodiment, the nanoparticle comprises a polymer. In one embodiment, the nanoparticle comprises a biodegradable polymer. In one embodiment, the polymer comprises polyesters of hydroxy carboxylic acids, polyanhydrides of dicarboxylic acids, or copolymers of hydroxy carboxylic acids and dicarboxylic acids. More generally, the polymer may comprise polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Polymers comprising ester and anhydride bonds (e.g., copolymers of glycolic and sebacic acid) may also be employed. For example, the polymer may comprise polystyrene, polyglycolic acid polymers (PGA), polylactic acid polymers (PLA), polysebacic acid polymers (PSA), polylactic-co-glycolic) acid copolymers (PLGA or PLG; the terms are interchangeable), [rho]oly(lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers comprise copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the polymer may comprise biologically important amino acids with reactive side chain groups, such as lysine, arginine, aspartic acid, glutamic acid, serine, threonine, tyrosine and cysteine, or their enantiomers, may be included in copolymers with any of the above. The polymer may also comprise non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons.

In one embodiment, the polymer comprises poly(DL-lactide) (PLA). In one embodiment, the PLA is acid-terminated. In one embodiment, the number molecular weight of PLA is from about 0.1 kD to about 75 kD, about 0.1 kD to about 70 kD, about 0.1 kD to about 65 kD, about 0.1 kD to about 60 kD, about 0.1 kD to about 55 kD, about 0.1 kD to about 50 kD, about 0.1 kD to about 45 kD, about 0.1 kD to about 40 kD, about 0.1 kD to about 35 kD, about 0.1 kD to about 30 kD, about 0.1 kD to about 25 kD, about 0.1 kD to about 20 kD, about 1 kD to about 20 kD, or about 5 kD to about 15 kD.

In one embodiment, the conjugate and the polymer form a core of the nanoparticle.

In one embodiment, the nanoparticle comprises a surfactant. The surfactant can be any surfactant known to a person of skill in the art. In one embodiment, the surfactant comprises a poloxamer, a poloaxamine, PEG, Tween-80, gelatin, dextran, pluronic L-63, PVA, methylcellulose, lecithin, DMAB, sodium deoxycholate, poly(acrylic acid), hyaluronic acid, vitamin E TPGS (D-α-tocopheryl polyethylene glycol 1000 succinate), or poly(ethylene-alt-maleic anhydride) (PEMA). In one embodiment, the surfactant is an anionic surfactant.

In one embodiment, the surfactant comprises PEMA. In one embodiment, the surfactant is PEMA. In one embodiment, the weight average molecular weight of PEMA is from about 10 kD to about 500 kD, about 30 kD to about 500 kD, about 50 kD to about 500 kD, about 70 kD to about 500 kD, about 90 kD to about 500 kD, about 110 kD to about 500 kD, about 130 kD to about 500 kD, about 150 kD to about 500 kD, about 200 kD to about 500 kD, about 250 kD to about 500 kD, about 300 kD to about 500 kD, about 300 kD to about 450 kD, about 350 kD to about 450 kD, or about 400 kD. In one embodiment, the PEMA is Vertellus E60 and/or E400.

In one embodiment, conjugate forms a core of the nanoparticle which is partially or completely coated by PEMA. In one embodiment, the conjugate and the PLA form a core which is partially or completely coated by PEMA.

In one embodiment, the loading of the drug moiety in the nanoparticle is from about 0.5 μg/mg to about 70 μg/mg. In one embodiment, the loading of the drug moiety in the nanoparticle is from about 2.5 μg/mg to about 5.0 μg/mg or from about 3.0 μg/mg to about 3.5 μg/mg. In another embodiment, the loading of the drug moiety in the nanoparticle is from about 8.0 μg/mg to about 12 μg/mg or from about 9.5 μg/mg to about 10.5 μg/mg. In another embodiment, the loading of the drug moiety in the nanoparticle is from about 25 μg/mg to about 35 μg/mg or about 29 μg/mg to 30 μg/mg. In another embodiment, the loading of the drug moiety in the nanoparticle is from about 55 μg/mg to about 65 μg/mg or about 62 μg/mg to 63 μg/mg. In one embodiment, the loading of the drug moiety was determined from the coupling efficiency via ¹H NMR. In one embodiment, the coupling efficiency was determined by calculating the ratio of methylene protons of PLGA to those of the phenyl protons associated with the compound of Formula (I).

In one embodiment, the average particle size of a plurality of nanoparticles is from about 50 nm to about 900 nm, about 150 nm to about 900 nm, about 300 nm to about 900 nm, about 450 nm to about 900 nm, about 550 nm to about 900 nm, about 600 nm to about 900 nm, about 600 nm to about 850 nm, about 650 nm to about 850 nm, or about 650 nm to about 800 nm.

In one embodiment, the polydispersity index (PDI) of a plurality of nanoparticles is from about 0.05 to about 0.6, about 0.1 to about 0.6, about 0.15 to about 0.55, about 0.15 to about 0.45, about 0.2 to about 0.4, about 0.2 to about 0.35, or about 0.2 to about 0.32. In one embodiment, PDI describes the width or spread of the size distribution of the plurality of nanoparticles. In one embodiment, the numerical value of PDI ranges from 0.0 (for a perfectly uniform sample with respect to the particle size) to 1.0 (for a highly polydisperse sample with multiple particle size populations). In one embodiment, PDI=the square of the standard deviation÷by the mean diameter of the nanoparticles.

In one embodiment, the nanoparticle has a zeta potential from about −20 mV to about −100 mV, about −20 mV to about −70 mV, about 0 mV to about −70 mV, or about −20 mV to about −49 mV.

### Compositions

In yet another aspect, the present disclosure provides a composition comprising a compound of Formula (I) or a salt thereof, a conjugate of Formula (10) or a salt thereof, or a nanoparticle comprising a conjugate of Formula (10) or a salt thereof. In one embodiment, the composition comprises a pharmaceutically acceptable carrier. In one embodiment, the pharmaceutically acceptable carrier comprises a solvent. In one embodiment, the solvent is an aqueous solvent. In one embodiment, the solvent is saline.

### Methods of Treatment

In another aspect, the present disclosure provides a method of treating a disease or disorder in a subject, the method comprising administering to the subject a compound of Formula (I) or a salt thereof, a conjugate of Formula (10) or a salt thereof, or a nanoparticle comprising a conjugate of Formula (10) or a salt thereof. In one embodiment, the disease or disorder is an inflammatory disease or disorder or a disease or disorder with a significant inflammatory component. In one embodiment, the disease or disorder is selected from psoriasis, autoimmunity, endometriosis, inflammatory bowel disease, sepsis, septic shock, hemorrhagic shock, or poly-trauma. In one embodiment, the disease or disorder is sepsis.

In one embodiment, the disease or disorder is treated by reducing an inflammatory response in the subject. In one embodiment, the disease or disorder is treated by inhibition of the NF-kB P38 MAPK signaling pathway in the subject. In one embodiment, inhibition of the NF-kB P38 MAPK signaling pathway treats the disease or disorder by decreasing proinflammatory cytokine levels in the subject, resulting in a reduced inflammatory response. In one embodiment, the disease or disorder is treated by inhibition of one or more HDACs in the subject. In one embodiment, HDACis at least one of HDAC1, HDAC2, HDAC3, HDAC6, and HDAC8.

In another aspect, the disclosure provides a method for delivering a compound of Formula (I) or a salt thereof to a subject, the method comprising administering to the subject a compound of Formula (I) or a salt thereof, a conjugate of Formula (10) or a salt thereof, or a nanoparticle comprising a conjugate of Formula (10) or a salt thereof. In one embodiment, the administration of the compound of Formula (I) or a salt thereof, the conjugate of Formula (10) or a salt thereof, or the nanoparticle comprising a conjugate of Formula (10) treats a disease or disorder in the subject. In one embodiment, the disease or disorder is selected from sepsis, septic shock, hemorrhagic shock, or poly-trauma. In one embodiment, the disease or disorder is sepsis.

In one embodiment, the compound of Formula (I) or salt thereof is a drug moiety. In one embodiment, the conjugate or nanoparticle releases a drug moiety of Formula (I) or a pharmaceutically acceptable salt thereof. In one embodiment, the drug moiety comprises SAHA or a pharmaceutically acceptable salt thereof. In one embodiment, SAHA is a non-selective HDAC inhibitor. In one embodiment, SAHA inhibits HDAC1, HDAC2, HDAC3, and HDAC6 in the subject. In another embodiment, the drug moiety comprises SAHA-OH or a pharmaceutically acceptable salt thereof. In one embodiment, SAHA-OH is a selective inhibitor of one HDAC. In one embodiment, SAHA-OH inhibits HDAC6.

In one embodiment, the drug moiety treats the disease or disorder by reducing an inflammatory response in the subject. In one embodiment, the drug moiety inhibits the NF-kB P38 MAPK signaling pathway in the subject. In one embodiment, the inhibition of the NF-kB P38 MAPK signaling pathway treats the disease or disorder by decreasing proinflammatory cytokine levels in the subject, resulting in a reduced inflammatory response. In one embodiment, the drug moiety inhibits one or more histone deacetylases (HDACs) in the subject. In one embodiment, HDACis at least one of HDAC1, HDAC2, HDAC3, HDAC6, and HDAC8.

In one embodiment, the compound of Formula (I) or salt thereof is formulated in a solution for administration to the subject. In another embodiment, the conjugate comprising a compound of Formula (10) or a salt thereof is formulated in a solution for administration to the subject. In another embodiment, the nanoparticle comprising the conjugate comprising a compound of Formula (10) or a salt thereof is formulated in a solution for administration to the subject. In one embodiment, the solution is an aqueous solution. In one embodiment, the solution comprises a pharmaceutically acceptable solvent. In one embodiment, the solvent is saline.

In one embodiment, the compound of Formula (I) or salt thereof is administered to the subject by injection. In another embodiment, the conjugate comprising a compound of Formula (10) or a salt thereof is administered to the subject by injection. In another embodiment, the nanoparticle comprising the conjugate comprising a compound of Formula (10) or a salt thereof is administered to the subject by injection.

In one embodiment, the compound of Formula (I) or salt thereof, the conjugate comprising a compound of Formula (10) or a salt thereof, or the conjugate comprising a compound of Formula (10) or a salt thereof is administered to the subject in combination with one or more further therapeutic agents. In one embodiment, the one or more further therapeutic agents are selected from antibiotics, analgesics, steroids, vasopressors, and vasoconstrictors.

In one embodiment, the subject is a human subject.

### Clauses of the Disclosure

The following clauses describe certain embodiments.

Clause 1. A compound of Formula (I)

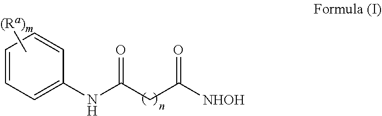

or a pharmaceutically acceptable salt thereof, wherein in Formula (I):
m is 0 or 1;
n is an integer selected from 1, 2, 3, 4, 5, 6, 7, and 8; and
R^{a}, when present, comprises one or more of —OH, C₁-C₃ alkyl, C₁-C₃ alkoxy, —C(O)H, —C(O)OH, —C(O)X; and
X is halogen selected from F, Br, Cl, and I.

Clause 2. The compound of clause 1, wherein n is 6.

Clause 3. The compound of clause 1 or 2, wherein suberoylanilide hydroxamic acid (SAHA):

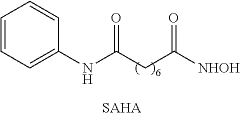

is excluded from Formula (I).

Clause 4. The compound of clause 1, wherein m is 1 and n is 6.

Clause 5. The compound of clause 4, wherein the compound of Formula (I) is N¹-hydroxy-N⁸-(4-(hydroxymethyl)phenyl)octanediamide (SAHA-OH):

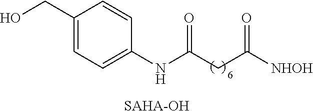

or a pharmaceutically acceptable salt thereof.

Clause 6. A conjugate of Formula (10), comprising a polymer moiety (POLY) covalently linked to a drug moiety comprising the compound of any one of clauses 1-5:

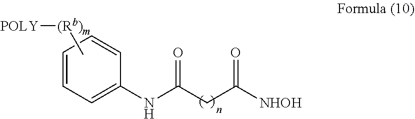

or a pharmaceutically acceptable salt thereof, wherein in Formula (10):
m is 0 or 1;
n is an integer selected from 1, 2, 3, 4, 5, 6, 7, and 8; and
R^{b}, when present, comprises one or more of —O—, —C₁-C₃ alkyl-, —C₁-C₃ alkoxy-, —C(O)—, —C(O)O—.

Clause 7. The conjugate of clause 6, wherein m is 0 and n is 6.

Clause 8. The conjugate of clause 6, wherein the conjugate of Formula (10) is

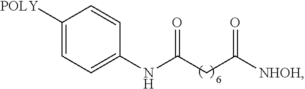

or a pharmaceutically acceptable salt thereof.

Clause 9. The conjugate of clause 6, wherein m is 1 and n is 6.

Clause 10. The conjugate of clause 6, wherein the conjugate of Formula (10) is

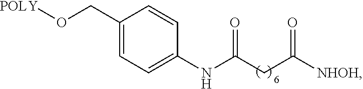

or a pharmaceutically acceptable salt thereof.

Clause 11. The conjugate of clause 6, wherein the conjugate of Formula (10) is

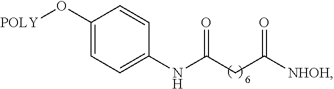

or a pharmaceutically acceptable salt thereof.

Clause 12. The conjugate of any one of clauses 6-11, wherein POLY is a biodegradable polymer.

Clause 13. The conjugate of any one of clauses 6-11, wherein POLY is selected from polyglycolic acid (PGA), poly(DL-lactide) (PLA), and poly(D,L-lactide-co-glycolide) (PLGA).

Clause 14. The conjugate of clause 13, wherein POLY is PLGA.

Clause 15. The conjugate of clause 14, wherein PLGA comprises lactic acid monomer subunits and glycolic acid monomer subunits in a ratio from about 25:75 to about 75:25 lactic acid monomer subunits:glycolic acid monomer subunits.

Clause 16. The conjugate of clause 14, wherein PLGA comprises lactic acid monomer subunits and glycolic acid monomer subunits in a ratio of from about 25:75 to about 50:50 lactic acid monomer subunits:glycolic acid monomer subunits.

Clause 17. The conjugate of clause 14, wherein PLGA comprises lactic acid monomer subunits and glycolic acid monomer subunits in a ratio of from about 50:50 to about 75:25 lactic acid monomer subunits:glycolic acid monomer subunits.

Clause 18. The conjugate of clause 14, wherein PLGA comprises lactic acid monomer subunits and glycolic acid monomer subunits in a ratio of about 25:75, about 50:50, or about 75:25 lactic acid monomer subunits:glycolic acid monomer subunits.

Clause 19. The conjugate of any one of clauses 13-18, wherein the PLGA comprises an acid terminus which is covalently linked to the drug moiety to form the conjugate of Formula (10).

Clause 20. The conjugate of any one of clauses 14-19, wherein POLY is a random copolymer, or has a formula

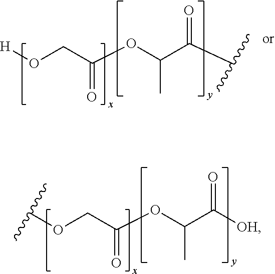

wherein each x and y is independently an integer from 1 to 10,000.

Clause 21. The conjugate of any one of clauses 6-20, wherein POLY has a number average molecular weight from about 3 kD to about 50 kD.

Clause 22. A nanoparticle comprising a conjugate of any one of clauses 6-21.

Clause 23. The nanoparticle of clause 22, further comprising poly(DL-lactide) (PLA).

Clause 24. The nanoparticle of clause 23, wherein the PLA is acid-terminated.

Clause 25. The nanoparticle of clause 23 or 24, wherein the number average molecular weight of PLA is from about 5 kD to about 50 kD.

Clause 26. The nanoparticle of any one of clauses 22-25, further comprising a surfactant.

Clause 27. The nanoparticle of clause 26, wherein the surfactant comprises a poloxamer, a poloaxamine, PEG, Tween-80, gelatin, dextran, pluronic L-63, PVA, methylcellulose, lecithin, DMAB, sodium deoxycholate, poly(acrylic acid), hyaluronic acid, vitamin E TPGS (D-α-tocopheryl polyethylene glycol 1000 succinate), or poly(ethylene-alt-maleic anhydride) (PEMA).

Clause 28. The nanoparticle of clause 27, wherein the surfactant is PEMA.

Clause 29. The nanoparticle of clause 28, wherein the weight average molecular weight of PEMA is from about 30 kD to about 500 kD.

Clause 30. The nanoparticle of clause 28 or 29, wherein the number average molecular weight of PEMA is about 400 kD.

Clause 31. The nanoparticle of any one of clauses 23-30, wherein the conjugate and PLA form a core.

Clause 32. The nanoparticle of clause 31, wherein the core is partially or completely coated by poly(ethylene-alt-maleic anhydride) (PEMA).

Clause 33. The nanoparticle of any one of clauses 22-32, wherein the loading of the drug moiety in the nanoparticle is from about 0.5 μg/mg to about 70 μg/mg.

Clause 34. The nanoparticle of any one of clauses 22-32, wherein the loading of the drug moiety in the nanoparticle is from about 2.5 μg/mg to about 5.0 μg/mg.

Clause 35. The nanoparticle of any one of clauses 22-32, wherein the loading of the drug moiety in the nanoparticle is from about 8.0 μg/mg to about 12 μg/mg.

Clause 36. The nanoparticle of any one of clauses 22-32, wherein the loading of the drug moiety in the nanoparticle is from about 25 μg/mg to about 35 μg/mg.

Clause 37. The nanoparticle of any one of clauses 22-32, wherein the loading of the drug moiety in the nanoparticle is from about 55 μg/mg to about 65 μg/mg.

Clause 38. A plurality of the nanoparticles of any one of clauses 22-37, wherein the average particle size of the plurality of nanoparticles is from about 50 nm to about 800 nm.

Clause 39. A plurality of the nanoparticles of any one of clauses 22-38, wherein the polydispersity index (PDI) of the plurality of nanoparticles is from about 0.05 to about 0.45.

Clause 40. A plurality of the nanoparticles of any one of clauses 22-39, having a zeta potential from about 0 mV to about −70 mV.

Clause 41. A pharmaceutical composition comprising the compound of any one of clauses 1-5, the conjugate of any one of clauses 6-21, or the plurality of nanoparticles of any one of clauses 22-40, and a pharmaceutically acceptable carrier.

Clause 42. A method of treating a disease or disorder in a subject, the method comprising administering to the subject the compound of any one of clauses 1-5, the conjugate of any one of clauses 6-21, the plurality of nanoparticles of any one of clauses 22-40, or the pharmaceutical composition of clause 41.

Clause 43. The method of clause 42, wherein the disease or disorder is an inflammatory disease or disorder or a disease or disorder with a significant inflammatory component.

Clause 44. The method of clause 42 or 43, wherein the disease or disorder is selected from psoriasis, autoimmunity, endometriosis, inflammatory bowel disease, sepsis, septic shock, hemorrhagic shock, and poly-trauma.

Clause 45. The method of clause 44, wherein the disease or disorder is sepsis.

Clause 46. The method of any one of clauses 42-45, wherein each of the compound, the conjugate, the plurality of nanoparticles, and the pharmaceutical composition is independently formulated in a solution.

Clause 47. The method of clause 46, wherein the solution is administered to the subject by injection.

Clause 48. The method of any one of clauses 42-47, wherein the compound, the conjugate, the plurality of nanoparticles, or the pharmaceutical composition administered to the subject in combination with one or more further therapeutic agents.

Clause 49. The method of clause 48, wherein the one or more further therapeutic agents are selected from antibiotics, analgesics, steroids, vasopressors, and vasoconstrictors.

Clause 50. The method of any one of clauses 42-49, wherein the subject is a human subject.

### EXAMPLES

The following non-limiting examples are provided to further illustrate embodiments of the disclosure disclosed herein. It should be appreciated by those of skill in the art that the techniques disclosed in the examples that follow represent approaches that have been found to function well in the practice of the disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the disclosure.

### Example 1. Modified Suberoylanilide Hydroxamic Acid Reduced Drug-Associated Immune Cell Death and Organ Damage Under Lipopolysaccharide Inflammatory Challenge

### Introduction

Suberoylanilide hydroxamic acid (SAHA; vorinostat), a pan-HDACi, has received approval from the Food and Drug Administration (FDA) for the treatment of cutaneous T cell lymphoma. The mechanism by which SAHA induces tumor cell death is via the regulation of proapoptotic and anti-apoptotic genes that results in activated proapoptotic pathways. While beneficial in a tumor environment, this apoptosis-mediated cytotoxicity is also their Achilles' heel, causing growth arrest and cell death in healthy immune cells due to lack of targeted delivery. The pleiotropic effects of pan-HDACis, like SAHA, are due non-specific inhibition of multiple classes of HDAC enzymes and non-histone proteins. Consequentially, this can often result in off-target cytotoxicity, including thrombocytopenia and neutropenia, as well as other adverse effects like cardiac abnormalities. The cytotoxicity of SAHA has served as a deterrent for its use to treat diseases where long-term therapy will be required.

Designing isoform-selective therapeutics could potentially mitigate drug-associated toxicity to promote its clinical use for applications outside of cancer. In fact, several studies have established the anti-inflammatory activity of SAHA through the suppression of proinflammatory cytokine production following lipopolysaccharide (LPS) challenge in vitro and in vivo. The reduction in proinflammatory cytokine secretions and the epigenetic regulation of gene expression serve as an attractive strategy to be targeted therapeutically. SAHA, as a sepsis therapeutic, is less favorable due to its potent immune cell cytotoxicity, despite its beneficial anti-inflammatory effects. Thus, strategies to reduce the off-target toxicity of SAHA, while promoting its anti-inflammatory properties are of significant clinical interest.

Several investigators have structurally modified HDACis in either the zinc-binding group (ZBG), the capping group, or the linker group to investigate its effect on drug selectivity and toxicity. Modifications at the capping group, such as bulky cap groups in compounds like Tubastatin A, can result in isoform-selective HDAC6 inhibition. Regarding substitutions to the capping groups of benzohydroxamic acids, Heimburg et al. showed that metabenzamidobenzohydroxamic acids resulted in HDAC8 selectivity due to binding at the surface ‘side pocket’ of HDAC8. Further, Goehringer et al. synthesized a parasubstitution to the capping region of SAHA, specifically generating a pentafluorothio analog, and observed a reduction in apoptosis when evaluated in tumor cell lines. The clinical implication of utilizing pan-HDACis is non-specific inhibition of HDACs, which could result in increased toxicity and undesirable side effects, thus limiting its effective long-term clinical use. Thus, it is anticipated that the development of improved HDACis can reduce drug cytotoxicity and lower drug dosing requirements, while eliciting similar efficacy.

In this study, a modified form of SAHA, termed SAHA-OH, was synthesized and characterized. SAHA-OH displayed a reduction in HDAC1, 2, 3, 8 isoform selectivity and a slightly increased HDAC6 isoform selectivity compared to SAHA. Using primary macrophages, the effects of SAHA versus SAHA-OH were directly compared on modulating LPS-stimulated primary macrophage cytokine secretions. Although similar reductions in proinflammatory cytokines were measured, distinct differences were revealed that further highlighted the improved biological properties of SAHA-OH for the modulation of inflammation. SAHA-OH displayed a significantly improved toxicity profile using primary macrophages and splenic B cells compared to SAHA, which was highly toxic. Using an LPS-induced endotoxemia mouse model of severe inflammation, similar significant reductions in plasma proinflammatory cytokine levels were achieved for SAHA and SAHA-OH. Importantly, SAHA-OH treatment mitigated LPS-induced organ damage, whereas profound disruption of organ architecture was observed for SAHA. The improved toxicity profile of SAHA-OH in addition to the retention of its anti-inflammatory properties in vitro and in vivo compared to SAHA provides evidence for the further evaluation of SAHA-OH for the treatment of severe inflammatory diseases and conditions.

### Methods

### Materials

LPS from Escherichia Coli serotype 0111:B4 (Cat #L2630) and RPMI 1640 supplemented with L-glutamine (Cat #R8758) was obtained from Sigma-Aldrich (St. Louis, MO). Penicillin-streptomycin, Versene, and NuPAGE™ 12% Bis-Tris 1.0 mm Mini Protein Gel (Cat #NPO343BOX) were purchased from Thermo Fisher Scientific (Waltham, MA). Fetal bovine serum (FBS) was purchased from VWR (Radnor, PA). L929 cells were purchased from ATCC (Manassas, VA). Acetyl-α-Tubulin (Lys40) (D20G3) Rabbit mAb (#5335), α-Tubulin (11H10) Rabbit mAb (#2125), Acetyl-histone H3 (Lys 9/Lys 14) (#9677) Rabbit mAb, Histone H3 (D1H2) (#4499) Rabbit mAb, and β-Actin (D6A8) (#8457) were purchased from Cell Signaling Technology (Danvers, MA). 2×SDS/PAGE sample buffer was produced using 4% SDS, 5.7 M P-mercaptoethanol, 0.2 M Tris/HCl, pH 6.8, 20% glycerol, and 5 mM EDTA. All chemical supplies were of analytical grade and obtained from Sigma-Aldrich (St. Louis, MO) and CombiBlocks (San Diego, CA) if not mentioned otherwise.

SAHA Precursor: Methyl 8-oxo-8-(phenyiamino)octanoate (SF-7-277)

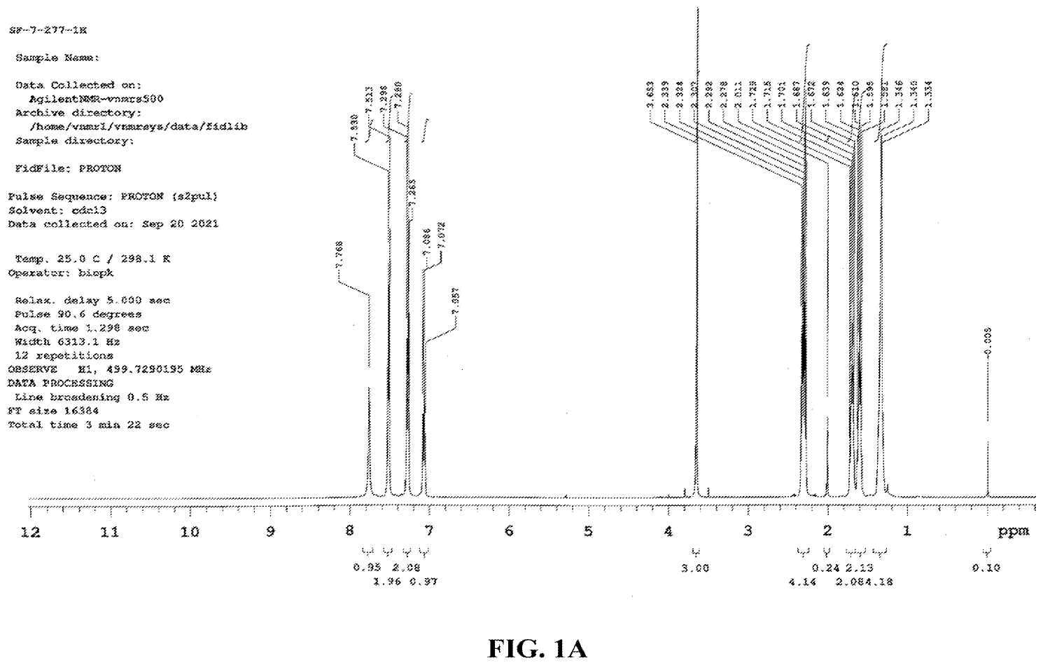

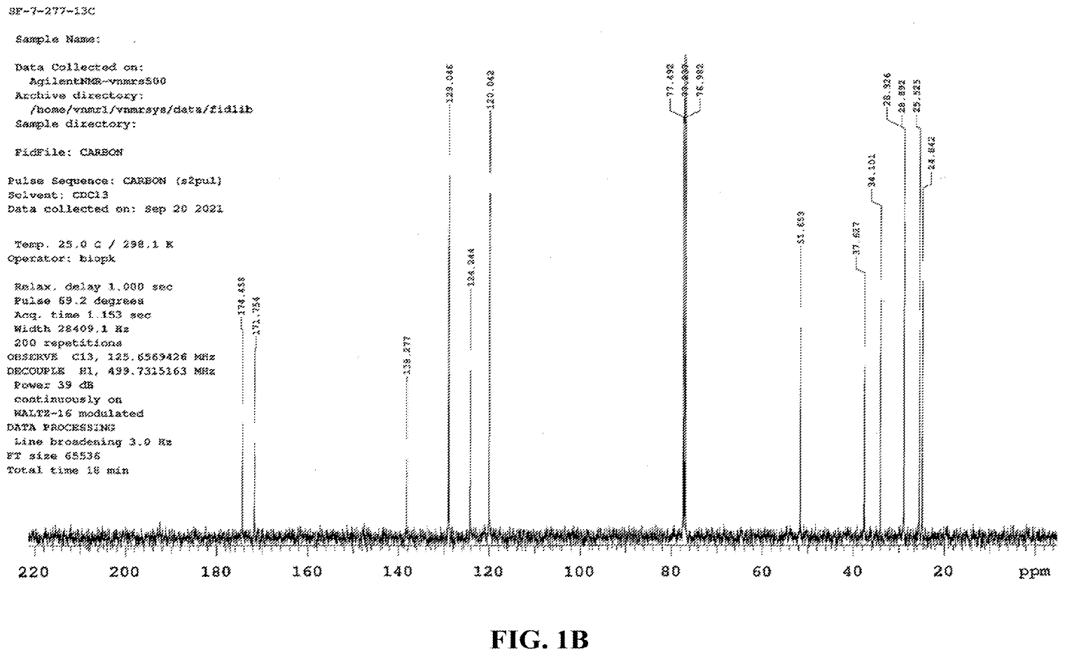

Aniline (132 mL, 1.45 mmol, 1 eq) and monomethyl suberate (299 mg, 1.59 mmol, 1.1 eq) were dissolved in anhydrous CH₂Cl₂ (15 mL). Subsequently, EDCI (305 mg, 1.59 mmol, 1.1 eq) and DMAP (18 mg, 0.145 mmol, 0.1 eq) were added. The reaction was allowed to stir at room temperature for 16 h. The next day, TLC confirmed the reaction was complete. The CH₂Cl₂ was further diluted, washed with 1 M HCl, saturated NaHCO₃, brine, dried (Na₂SO₄), filtered, and concentrated. The crude residue was adsorbed to silica gel, then purified by flash column chromatography (gradient of ethyl acetate in hexanes) to deliver the title compound as a white solid (291 mg, 79%). ¹H NMR (500 MHz, CDCl₃-d): δ (ppm) 7.77 (s, 1H), 7.52 (d, J=8.5 Hz, 2H), 7.28 (t, J=7.8 Hz, 2H), 7.07 (t, J=7.3 Hz, 1H), 3.65 (s, 3H), 2.31 (m, J=7.6 Hz, 4H), 1.70 (t, J=7 Hz, 2H), 1.61 (t, J=7.3 Hz, 2H), 1.34 (t, J=3 Hz, 4H) (FIG. 1A). ¹³C NMR (500 MHz, CDCl₃-d): δ (ppm) 174.5, 171.8. 138.3, 129.1, 124.2, 120.0, 77.5, 77.0, 51.7, 37.6, 34.1, 28.9 (2), 25.5, 24.8. (FIG. 1i).

SAHA-OH Precursor: Methyl 8-((4-(hydroxymethyl)-phenyl)amino)-8-oxooctanoate (SF-7-258)

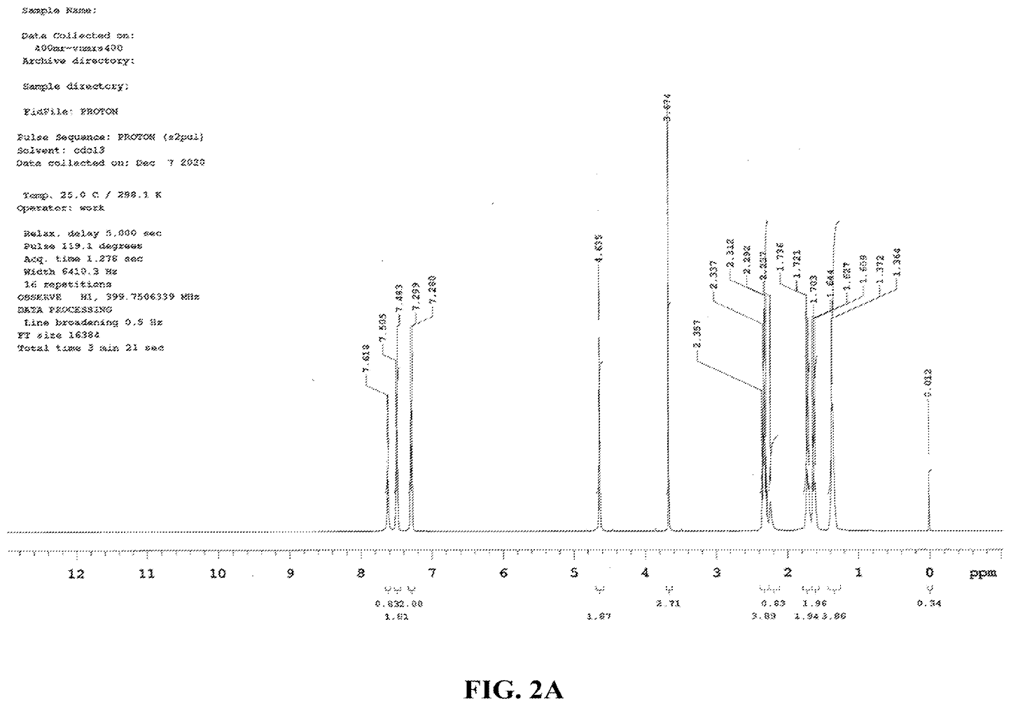

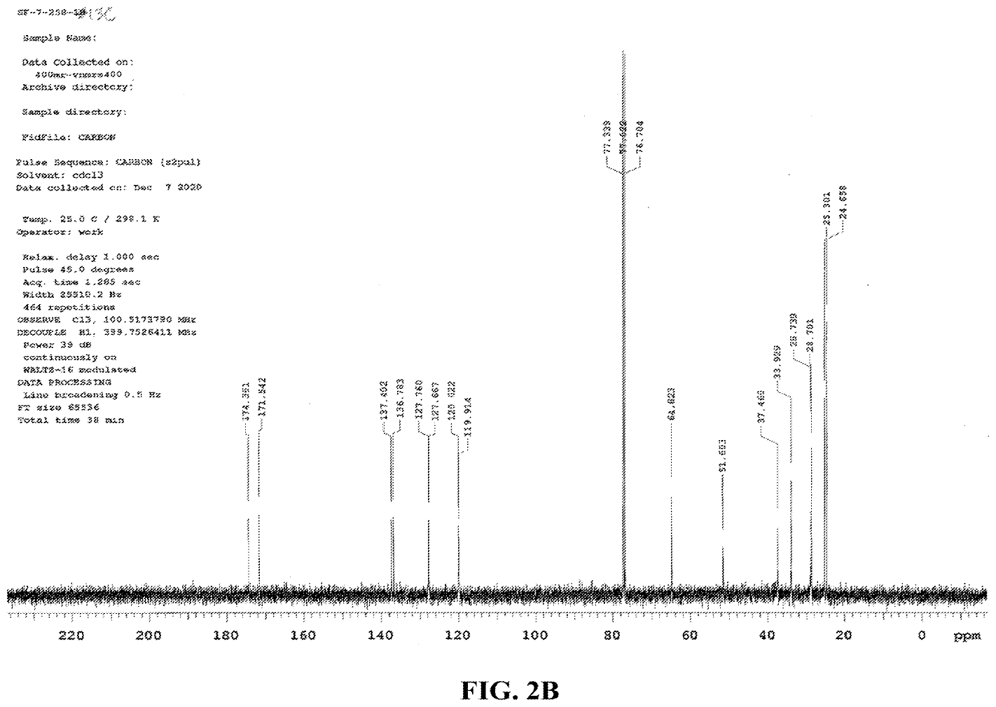

To a stirred solution of 4-aminobenzyl alcohol (327 mg, 2.66 mmol) in dimethylformamide (DMF) (13.0 mL), EDCI (586 mg, 3.06 mmol), hydroxybenzotriazole monohydrate (413 mg, 3.06 mmol), and monomethyl suberate (500 mg, 2.66 mmol) were added. The reaction mixture was stirred for 16 hours at 20° C. after which a thin layer chromatography (TLC) confirmed the reaction was complete. The reaction mixture was partitioned between water and ethyl acetate. The layers were separated, and the aqueous layer was extracted once more with ethyl acetate. Both organic layers were combined and then washed once using saturated sodium bicarbonate, three times using water, and a final time using brine. The organic layer was dried with anhydrous sodium sulfate, filtered, and concentrated under vacuum. The crude material was adsorbed to silica gel then and purified by flash column chromatography using a gradient of ethyl acetate in hexanes (6:1) to yield the product as a white solid (500 mg, 1.70 mmol, 64%). ¹H NMR (400 MHz, CDCl₃-d): δ (ppm) 7.62 (s, 1H), 7.49 (d, J=8.8 Hz, 2H), 7.29 (d, J=7.6 Hz, 2H), 4.64 (s, 2H), 3.67 (s, 3H), 2.36-2.29 (m, 4H), 1.72 (t, J=6 Hz, 2H), 1.63 (t, J=6.8 Hz, 2H) (FIG. 2A). ¹³C NMR (400 MHz, CDCl₃-d): δ (ppm) 174.4, 171.5, 137.4, 136.8, 127.7, 120.0, 64.8, 51.6, 37.5, 33.9, 28.7 (2), 25.3, 24.7 (FIG. 2B).

SAHA: N¹-hydroxy-N⁸-phenyloctanediamide (SF-7-278)

To a stirred 0° C. solution of hydroxylamine hydrochloride (158 mg, 2.27 mmol) and 5 M sodium methoxide solution (682 L, 3.41 mmol) in anhydrous methanol (2.0 mL) was added SF-7-277 (60 mg, 0.227 mmol). After 1 h, the reaction was complete which was confirmed by TLC. The reaction was gently acidified with 1 M HCl and then concentrated to dryness. The resulting crude product was adsorbed to silica gel and purified by flash column chromatography (10% methanol in dichloromethane) to yield the product as a beige solid (45 mg, 0.170 mmol, 75%).

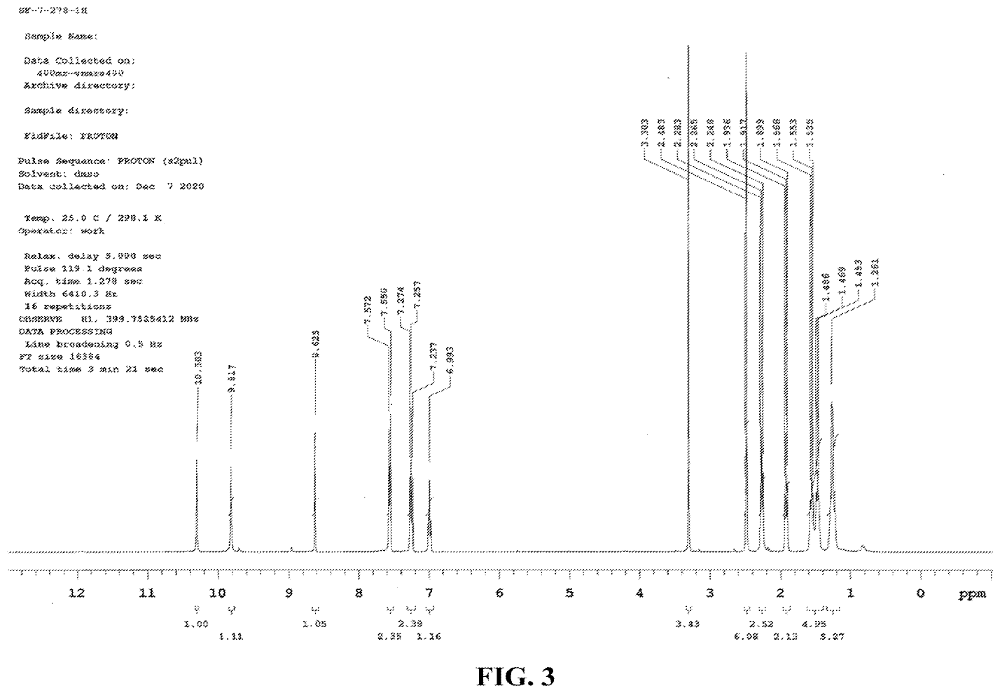

¹H NMR (400 MHz, DMSO-d₆): δ (ppm) 10.30 (s, 1H), 9.81 (s, 1H), 8.63 (s, 1H), 7.56 (d, J=8.8 Hz, 2H), 7.26 (t, J=6.8 Hz, 2H), 6.99 (t, J=6.8 Hz, 1H), 2.27 (t, J=7.2 Hz, 2H), 1.92 (t, J=7.6 Hz, 2H), 1.55 (t, J=6 Hz, 2H), 1.47 (t, J=6.8 Hz, 2H) (FIG. 3).

SAHA-OH: N¹-hydroxy-N⁸-(4-(hydroxymethyl)phenyl)-octanediamide (SF-7-279)

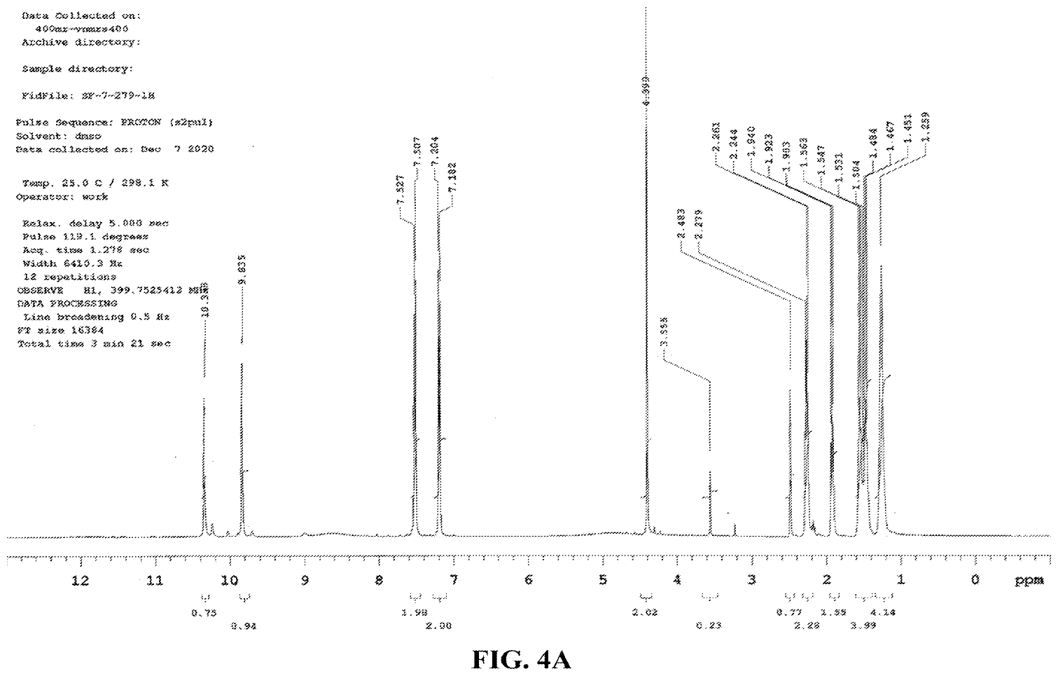

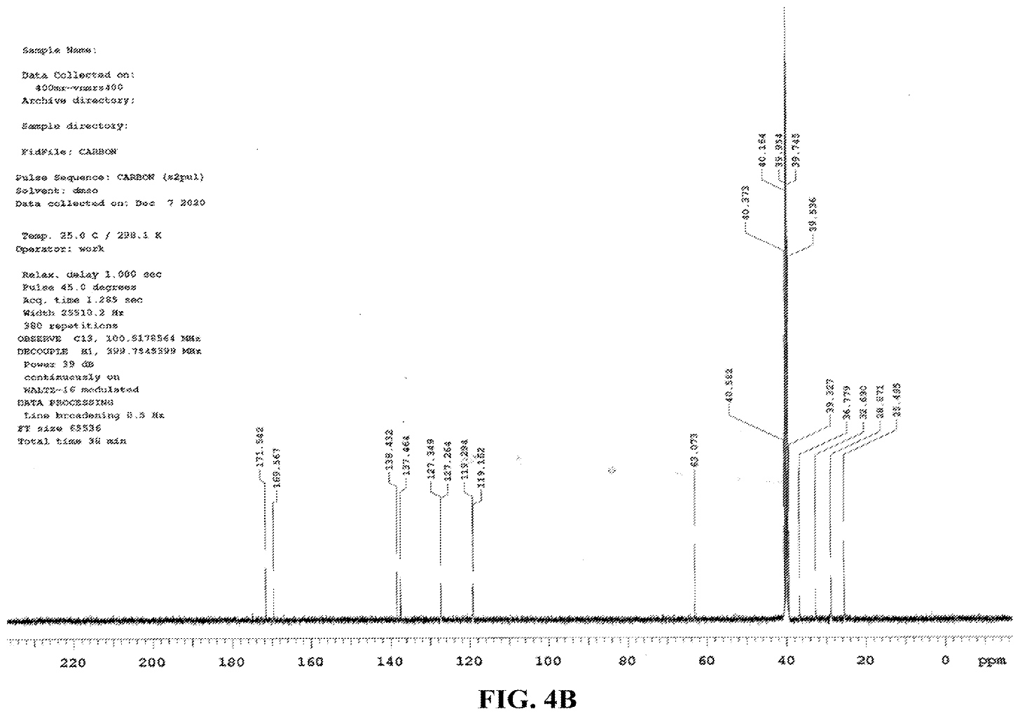

To a stirred solution of SF-2-258 (100 mg, 0.341 mmol) in methanol (5.0 mL) was added hydroxylamine hydrochloride (237 mg, 3.41 mmol) and 5 M sodium methoxide solution (1 mL, 5.12 mmol). The reaction mixture was stirred for 16 h at 20° C. after which a TLC confirmed the reaction was complete. The reaction was gently acidified with 1 M HCl and then concentrated to dryness. The resulting crude product was re-suspended in water (5 mL) and isolated as a white powder (50 mg, 0.170 mmol, 50%) by vacuum filtration while washing with water. ¹H NMR (400 MHz, DMSO-d₆): δ (ppm) 10.34 (s, 1H), 9.84 (s, 1H), 7.52 (d, J=8 Hz, 2H), 7.19 (d, J=8.8 Hz, 2H), 4.40 (s, 2H), 2.26 (t, J=7.2 Hz, 2H), 1.92 (t, J=6.8 Hz, 2H), 1.55 (t, J=6.4 Hz, 2H), 1.47 (t, J=6.8 Hz, 2H) (FIG. 4A). ¹³C NMR (400 MHz, DMSO-d₆): δ (ppm) 171.5, 169.6, 138.4, 137.5, 127.3, 119.2, 63.0, 40.0 (2 signals obsc by solvent), 36.8, 32.7, 28.9, 25.5 (FIG. 4B).

### IC₅₀ HDAC Selectivity

Fluorescence-based activity assays using fluorogenic peptides as substrates for HDAC1, 2, 3, 6, and 8 were performed by Reaction Biology Corporation (reactionbiology.com). A singlet 10 dose IC₅₀ mode with 2.5-fold serial dilution starting at 100 μM was prepared. The substrate for HDACs 1, 2, 3, and 6 was a fluorogenic peptide from p53 residues 379-382 (RHKK(Ac)AMC) (SEQ ID NO: 1). The substrate for HDAC8 was a fluorogenic peptide from p53 residues 379-382 (RHK(Ac)K(Ac)AMC) (SEQ ID NO: 2). SAHA-OH IC₅₀ values were calculated using GraphPad Prism 4 program based on a sigmoidal dose-response equation. The blank (DMSO) value was entered as 1.00×10⁻¹² of concentration for curve fitting. SAHA IC₅₀ values were obtained from Negmedin et al.

### Mice

Male and female C57BL/6J (5-7 weeks old) were purchased from The Jackson Laboratories (Bar Harbor, ME). The mice were housed under specific pathogen-free conditions in a facility. All mouse procedures and experiments were compliant with the protocols of the Institutional Animal Care and Use Committee (IACUC).

### Isolation and Generation of Bone Marrow-Derived Macrophages (BAIIØs)

BMMØs were generated from isolated bone marrow as previously described. Briefly, 5-12-week C57BL/6J female mice were euthanized and the femurs and tibias were isolated and flushed with BMMØ complete media (RPMI 1640 supplemented with L-glutamine, penicillin (100 units/mL), streptomycin (100 μg/mL), 10% heat-inactivated FBS, and 20% M-CSF in L929 cell-conditioned media) using a 1 mL syringe and a 25-gauge needle. Once isolated, the cells were pipetted and filtered through a 40 μm cell strainer and then plated in uncoated 10 cm non-tissue culture treated Petri dishes. The cells were incubated at 37° C. at 5% CO₂ and the BMMØ complete media was replaced on days 0, 3, 6, and 8. BMMØs were used for experiments between days 8-10 and were lifted using Versene. Cell number and viability were determined using trypan blue solution and EVE™ Automated Cell Counter (NanoEntek, Waltham, MA).

### BMMØ Cytokine Secretion Following LPS Stimulation

To evaluate BMMØ cytokine secretion, day 8 BMMØs were seeded at 1×10⁵ cells/well in complete BMMØ media in sterile 24-well plates incubated at 37° C. and 5% CO₂ overnight to allow for cell adherence. The following day, media was replaced with fresh complete media supplemented with the desired concentration of SAHA or SAHA-OH. Three hours later, cells were stimulated with 100 ng/mL LPS. After 48 hours, cell culture supernatants were collected and analyzed by enzyme-linked immunosorbent assays (ELISA) (BioLegend, San Diego, CA) to measure murine IL-6 and TNFα. MAGPIX Luminex bead-based multiplex ELISA (Thermo Fisher Scientific, Waltham, MA) measured for 7 various cytokines and chemokines, and data were analyzed using the Luminex xPONENT software (Millipore) as per manufacturer's instructions. The 7-plex panel included murine IFNβ, IL-10, IL-6, IL-1β, MCP-1 (CCL2), TNFα, and GROα (CXCL1).

### MTS Assay

The cytotoxicity of SAHA and SAHA-OH was evaluated using an MTS assay (Abcam; Cambridge, MA). Day 8 BMMØs were seeded at 1×10⁵ cells/well in complete BMMØ media in sterile 24-well plates incubated at 37° C. and 5% CO₂ overnight to allow for cell adherence. BMMØs were pre-treated with various concentrations of SAHA or SAHA-OH for 48 hours. Cells were then washed with PBS and 50 μL of MTS solution was added to each well and incubated for 1 hour. The optical density (O.D.) of the solution was measured using a SpectraMax iD3 microplate reader at 570 nm. The percentage of cell proliferation was measured as the ratio of O.D. at 570 nm to no treatment control. IC₅₀ values were calculated using GraphPad Prism 9 program based on a sigmoidal dose-response equation.

### Western Blot Analysis

Day 8 BMMØs were seeded at 1×10⁶ cells/well in sterile 6-well plates incubated at 37° C. and 5% CO₂ overnight to allow for cell adherence. BMMØs were treated with 10 μM of SAHA or 10 μM of SAHA-OH and incubated for 4 or 9 hours. Cells were lysed with Halt™ Protease Inhibitor Cocktail (Cat #78429) (Thermo Fisher, Waltham MA) in RIPA Buffer (Cat #R0278) (Sigma-Aldrich, St. Louis, MO) and scraped with a cell scraper (VWR, Radnor, PA). Lysates were sonicated with a ⅛^{th} needle tip using a Cole-Parmer 500-Watt Ultrasonic Homogenizer at 40% amplitude for 10 seconds on ice. Lysates were then centrifuged at 4° C. for 20 minutes at 12,000×g. The supernatant was collected and frozen at −80° C. until ready for western blot analysis. Protein lysates were produced using 50/50 sample to 2×SDS/PAGE sample buffer. Proteins were then separated by SDS/PAGE and immunoblotted using Histone H3 (D1H2) XP, Acetyl-Histone H3 (lys9/Lys14), α-Tubulin (11H10), Acetyl-α-Tubulin (Lys40) (D20G3) XP and β-Actin as primary antibodies. ECL was used for detection.

### BMMØ FITC Annexin V (AV) Apoptosis Detection Via Flow Cytometry

Day 8 BMMØs were seeded at 1×10⁵ cells/well in complete BMMØ media in sterile 24-well plates incubated at 37° C. and 5% CO₂ overnight to allow for cell adherence. The following day, media was replaced with fresh BMMØ complete media supplemented with SAHA or SAHA-OH (0-30 μM). Three hours later, cells were stimulated with 100 ng/mL LPS. After 48 hours, cells were lifted using Versene and washed with cold PBS supplemented with 1% FBS and 0.4% 0.5 M EDTA (Quality Biological, Gaithersburg, MD). FcR blocking was performed with purified anti-mouse CD16/32 antibody (#101301) (Biolegend, San Diego, CA) prior to staining with Annexin V-FITC and propidium iodide kit (#640914) (BioLegend, San Diego, CA) for apoptosis and cell death. Cell staining was conducted according to BioLegend protocols for flow cytometry. Flow cytometry data were collected using a Becton Dickinson LSR II or Becton Dickinson Canto II flow cytometer. Flow plots and data processing was done using FCS Express 7 Flow Cytometry De Novo Software (De Novo, Glendale, CA).

### Isolation and Culturing of Splenocytes for Apoptosis and Cell Death

Splenocytes were isolated from spleens as previously described. Briefly, 5-12-week C57BL/6J female mice were euthanized and the spleen was isolated. The spleen was processed into single-cell suspensions using a 40 μm cell strainer and flushed with cold PBS supplemented with 1% FBS and 0.4% 0.5 M EDTA (Quality Biological, Gaithersburg, MD). Erythrocytes were lysed with ACK lysis buffer (Quality Biological, Gaithersburg, MD). Splenocytes were cultured with splenocyte complete media (RPMI 1640 supplemented with L-glutamine, penicillin (100 units/mL), streptomycin (100 μg/mL), 10% heat-inactivated FBS, 1.7 μL β-mercaptoethanol (VWR Radnor, PA), concanavalin A (2 g/mL) (Sigma-Aldrich St. Louis, MO) and IL-7 (10 ng/mL) (BioLegend, San Diego, CA). Splenocytes were seeded at 2×10⁶ cells/well in a sterile 96 well plate. Cell number and viability were determined using trypan blue solution and EVE™ Automated Cell Counter (NanoEntek, Waltham, MA). The same day, media was replaced with fresh splenocyte complete media supplemented with SAHA or SAHA-OH (0-M) for 3 hours. Subsequently, splenocytes were stimulated with 100 ng/mL LPS for 24 hours. Splenocytes were then washed with cold PBS supplemented with 1% FBS and 0.4% 0.5 M EDTA and FcR blocked, as previously mentioned. Splenocytes were then stained with purified anti-mouse PE/Cyanine7 CD45R/B220 antibody (#103222) (BioLegend, San Diego, CA) for the B cell population. SAHA and SAHA-OH apoptosis and cell death were then assessed using the FITC-AV Apoptosis Detection Kit with PI via flow cytometry, as previously mentioned. Flow cytometry data were collected using a Cytex Aurora flow cytometer. Flow plots and data processing was done using FCS Express 7 Flow Cytometry De Novo Software (De Novo, Glendale, CA).

### Cytokine Analysis on the LPS-Induced Endotoxemia Mouse Model

LPS endotoxemia was induced using male C57BL/6J mice (5-7 weeks) (n=6) by i.p. injection with 50 mg/kg SAHA or SAHA-OH three hours prior to i.p. LPS injection (20 mg/kg). Terminal cardiac blood draws from the left ventricle occurred 3 hours post LPS injections using a 25 g needle and 1 mL syringe into an EDTA coated lavender blood collection tube (BD Medical, Franklin Lakes, NJ). Blood samples were centrifuged at 1000×g for 10 minutes within 30 minutes of collection. Plasma was extracted and diluted 10,000-fold for ELISA IL-6 measurement and diluted 10-fold for TNFα measurements.

### Hematoxylin and Eosin (H&E) Histological Sectioning

The differences in tissue architecture of liver and spleen were assessed using the same cohort of mice as the cytokine analysis at 3 hours post-LPS administration. Prior to tissue harvest, whole-body perfusion was performed post-cardiac blood draw by slowly flushing 10 mL of 37° C. PBS through the left ventricle of the heart while a cut in the right atrium allowed for the flow of blood. The liver and spleen were harvested, placed into tissue cassettes (VWR, Radnor, PA), and fixed overnight in 10% buffered formalin (Sigma-Aldrich, St. Louis, MO). The following day, the organs were paraffin-embedded and sectioned into 5 μm slices. Slides were stained for H&E to define histological architecture using standard procedures.

### Statistical Analysis

Statistical analyses were performed using Prism 9 (GraphPad, San Diego, CA). Results are reported as mean±standard deviation (SD). Student's t-test was used to determine the significance of parametric data between two groups. Significant differences between multiple treatment groups were determined by one-way ANOVA along with Tukey's multiple comparison test. Heatmap was plotted as Log 2 Fold change relative to LPS control. In all cases, unless otherwise noted, p<0.05 was considered to be statistically significant.

### Results

### Synthetic Modification of SAHA to Generate SAHA-OH

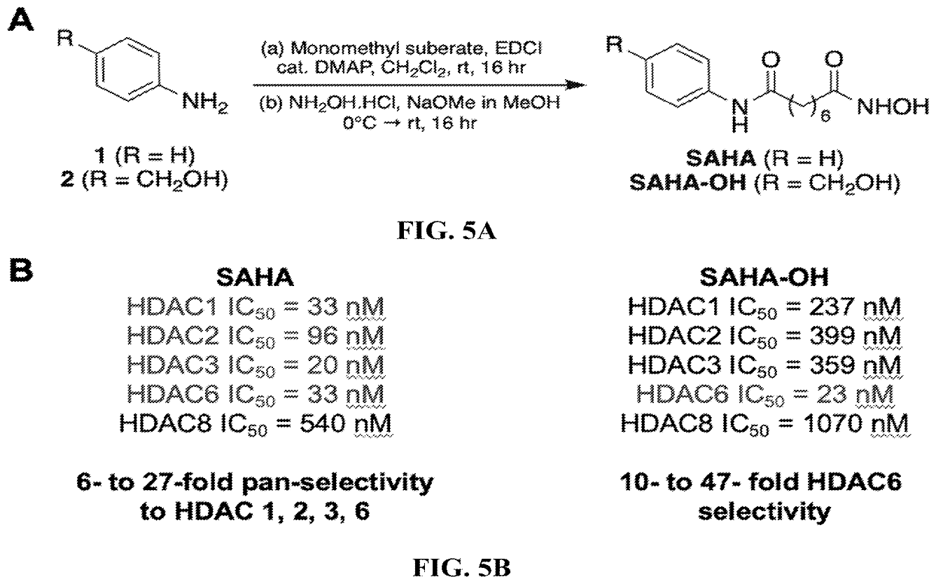

The HDACi pharmacophore comprises three segments: the capping group, the linker and the zinc-binding group (ZBG). Previous research into the expansion of HDACi libraries has indicated that the capping group is the most tolerant to modification, and the location of choice when grafting on a secondary pharmacophore to convert a selective HDACis into a dual inhibitor. Thus, the most studied HDACi, SAHA, was selected and a methine alcohol functionality (CH₂OH) was introduced into the para position of its phenyl ring as the grafting point to tether the HDACi via an ester linkage to the carboxylic acid of PLGA. SAHA was hydroxylated at its “capping” region, the part of the pharmacophore most tolerant to modifications, to generate SAHA-OH (FIG. 5A). A methine alcohol functionality (CH₂OH) was introduced onto the para position of the phenyl ring of SAHA. Briefly, SAHA and SAHA-OH were prepared similarly by first acylating 1 (FIGS. 1A-1B) or 2 (FIGS. 2A-2B), respectively, with monomethyl suberate. This was followed by a conversion of the methyl esters to hydroxamic acids with a solution of NH₂OH and NaOMe in MeOH to generate SAHA (FIG. 3) and SAHA-OH (FIGS. 4A-4B), respectively.

### HDAC Enzyme Binding Assays

To investigate how the modification to the capping group of SAHA affected its inhibition of HDAC enzymes, a series of fluorescence-based activity assays were performed against various classes of HDAC enzymes. IC₅₀ values against HDAC1, 2, 3, 6, and 8 were determined for SAHA-OH. Interestingly, SAHA-OH demonstrated a 10- to 47-fold selectivity against HDAC6, while SAHA demonstrated 6- to 27-fold pan-selectivity against HDAC1, 2, 3, 6 (FIG. 5B).

### SAHA-OH Retained Similar Anti-Inflammatory Properties as SAHA

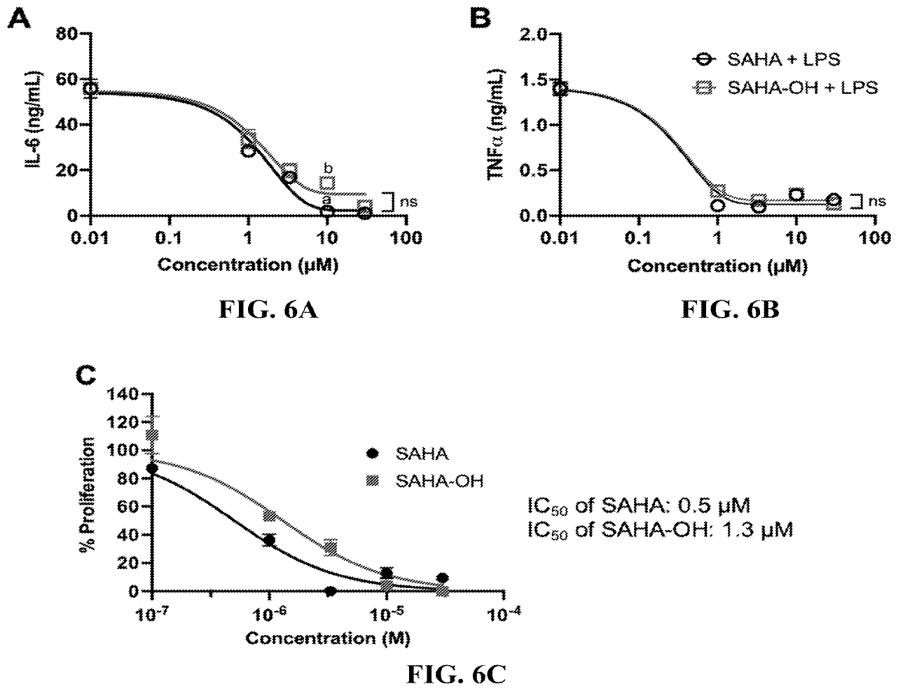

To determine if the modification of SAHA affected its anti-inflammatory properties, bone marrow macrophages (BMMØ) were treated with SAHA or SAHA-OH (0-30 μM) for 3 hours before the addition of LPS (100 ng/mL) for 48 hours. Subsequently, cell culture supernatants were collected and assayed using ELISA to measure cytokine secretions. Severe inflammation and sepsis results in immunosuppression brought on by reduced responsiveness to inflammatory signals and significant immune cell apoptosis. As described above, HDACis increase nuclear and cytoplasmic acetylation to reverse proinflammatory responses and protect cells from apoptosis. SAHA has demonstrated its ability to inhibit NF-κB activity in a dose-dependent manner. NF-κB inhibition can result in decreased proinflammatory cytokine generation to reduce the acute cytokine storm. FIGS. 6A-6B showed a dose response curve of SAHA and SAHA-OH treated bone marrow macrophages (BMMØ) resulted in decreased IL-6 and TNFα proinflammatory cytokine secretions after LPS stimulation. A similar concentration-dependent reduction in IL-6 and TNFα cytokine secretions was observed for SAHA and SAHA-OH (FIGS. 6A-6B). Interestingly, SAHA-OH treated at 10 μM was less effective than SAHA at suppressing IL-6 secretions. At all other concentrations tested, IL-6 and TNFα levels were not significantly different, demonstrating that the biological activity of SAHA was not affected appreciably by hydroxylation. SAHA and SAHA-OH IC₅₀ values in unstimulated BMMØs treated for 48 hours were determined utilizing an MTS assay, where an IC₅₀ of 0.5 μM and 1.3 μM were determined for SAHA and SAHA-OH, respectively (FIG. 6C). The increased IC₅₀ value for SAHA-OH indicated it was less cytostatic than SAHA.

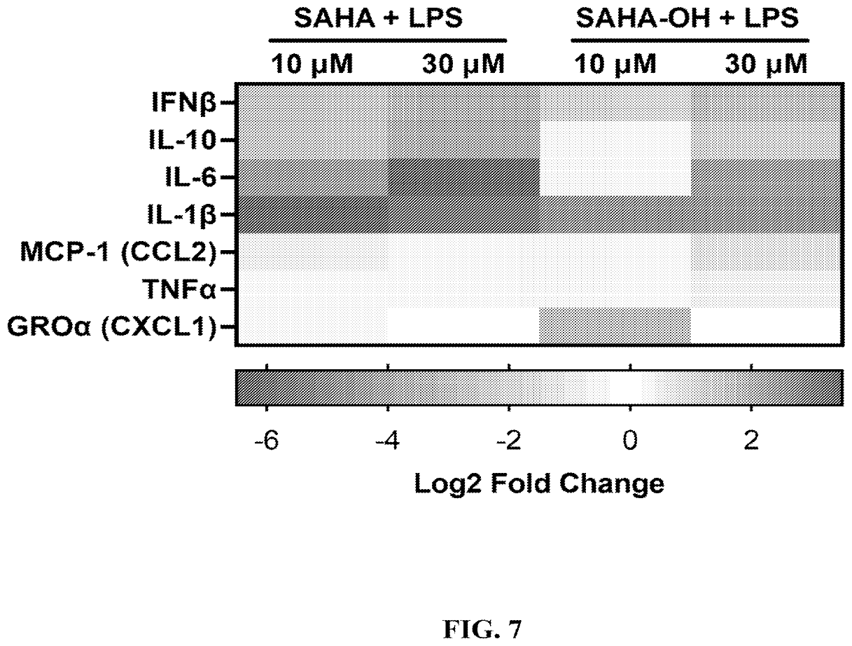

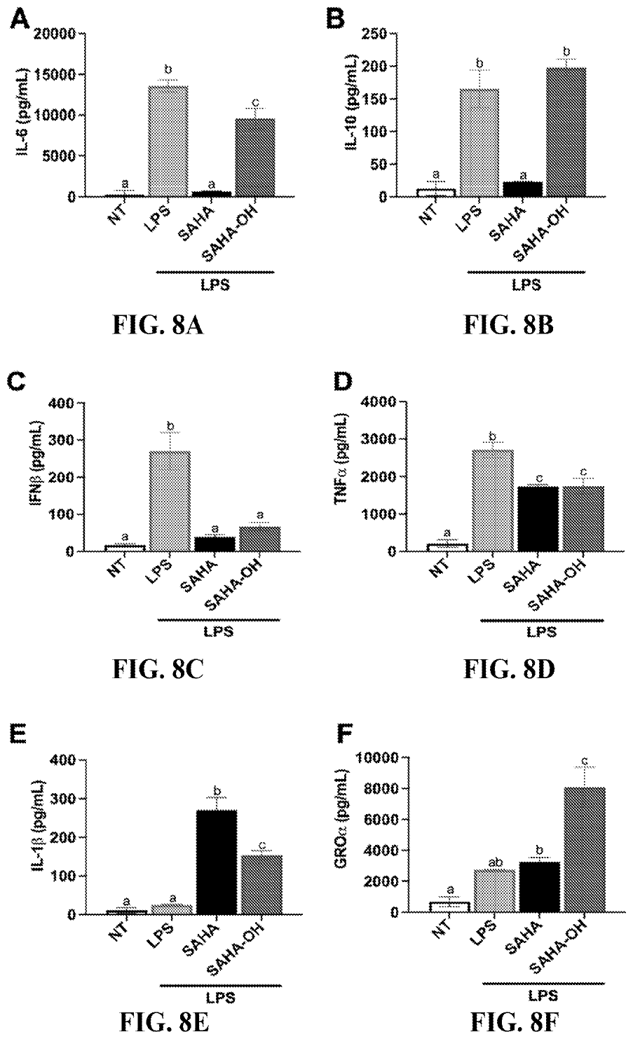

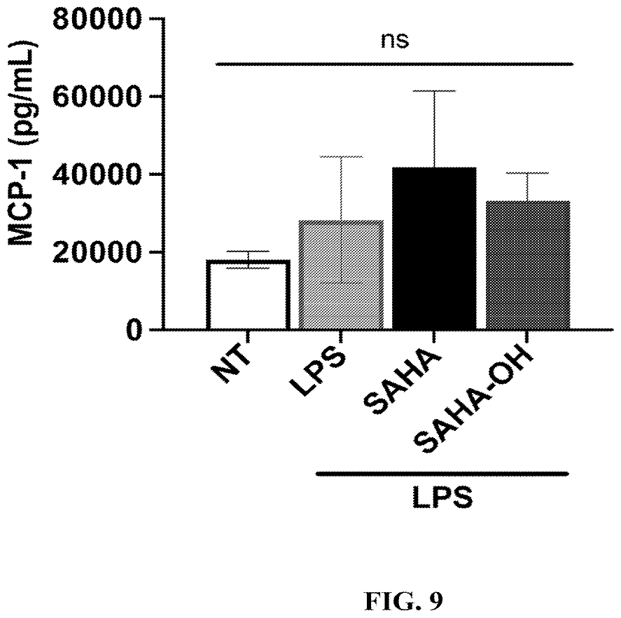

Further examination of additional cytokines and chemokines using a Luminex bead-based immunoassay was performed to identify other potential differences between SAHA and SAHA-OH cytokine profiles at two concentrations (10 μM and 30 μM) under LPS stimulation. SAHA and SAHA-OH at 30 μM demonstrated similar cytokine secretion profiles for all the proinflammatory cytokines (FIG. 7). Notably, 10 μM SAHA-OH only moderately suppressed IL-6 (FIG. 8A) and did not affect IL-1β (FIG. 8B) cytokine levels as effectively as 10 μM SAHA, relative to LPS control. For IFNβ (FIG. 8C) and TNFα (FIG. 8D), SAHA-OH similarly suppressed cytokine levels compared to SAHA, relative to LPS control. Previous investigations have established that reductions of IL-6, IL-10, IFNβ, and TNFα are associated with improved survival and therapeutic outcomes in mouse models of sepsis. The increased secretion of IL-10 (FIG. 8E) for SAHA compared to SAHA-OH treated BMMØs further complements the increased IC₅₀ values of SAHA-OH and its reduced cytostatic properties (FIG. 6C), as Bode et al. has demonstrated that HDACis promote IL-10 cleavage via a caspase-8-dependent mechanism in LPS-stimulated BMMØs. Interestingly, treatment with SAHA-OH led to a significant upregulation in GROα (CXCL1) compared to LPS or SAHA-treated BMMØs. Jin et al. demonstrated the role of GROα in increasing bacterial clearance through the recruitment of neutrophils at the site of infection to attenuate the effects of polymicrobial sepsis. SAHA and SAHA-OH non-significantly affected MCP-1 cytokine secretions (FIG. 9).

### SAHA-OH Treatment Induced Acetylation of Cytoplasmic α-Tubulin and to a Lesser Extent, Nuclear Histone H3

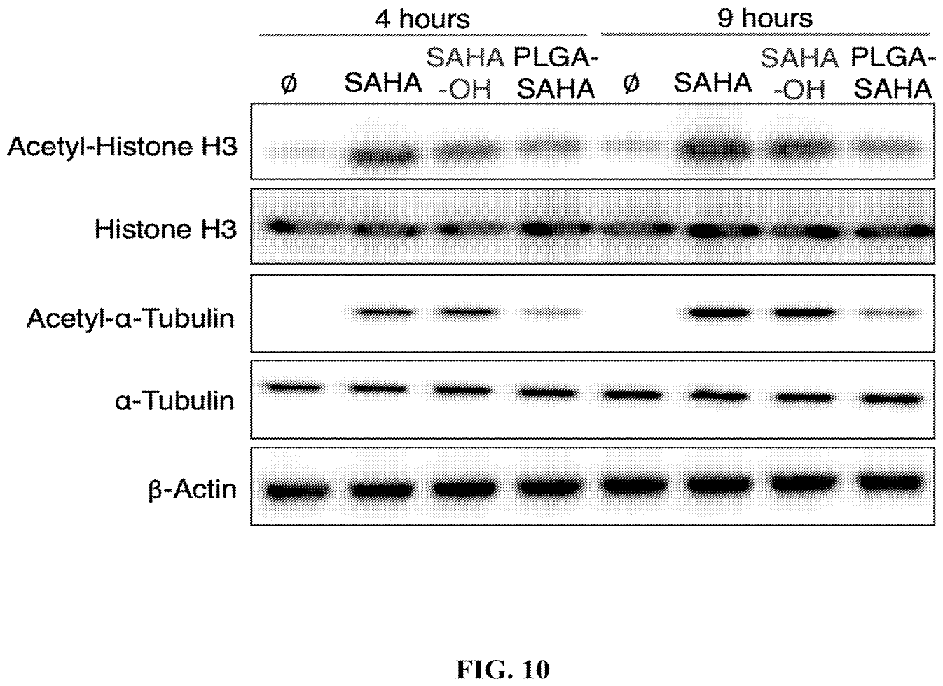

HDAC enzymes regulate the acetylation of histone proteins, such as histone H3, and non-histone proteins, like α-tubulin. To confirm that SAHA-OH inhibits HDAC enzymes, the acetylation of nuclear histone H3 and cytoplasmic α-tubulin were analyzed via western blotting (FIG. 10). Unstimulated BMMØs were treated with 10 μM SAHA or SAHA-OH at two time points, 4 and 9 hours. Cell lysates were collected and analyzed using antibodies against acetyl-histone H3 and acetyl-α-tubulin. Histone H3, α-tubulin, and 3-actin were used as internal controls to account for equal protein loading onto the gel. SAHA and SAHA-OH treatment similarly resulted in the acetylation of α-tubulin. Interestingly, SAHA-OH treatment led to a slight decrease in histone H3 acetylation compared to SAHA, but still induced the acetylation of histone H3 compared to no treatment (NT). These results indicate that SAHA-OH retains its HDACi functionality through the observed acetylation of histone H3 and α-tubulin.

### SAHA-OH Treatment Improved Cellular Viability and Attenuated Macrophage Apoptosis and B Cell Death

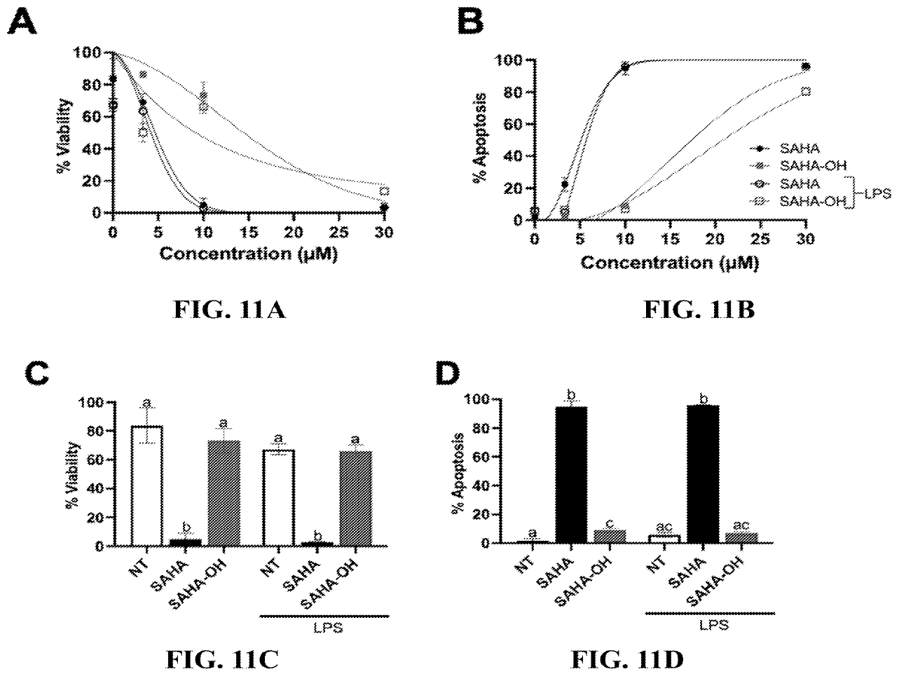

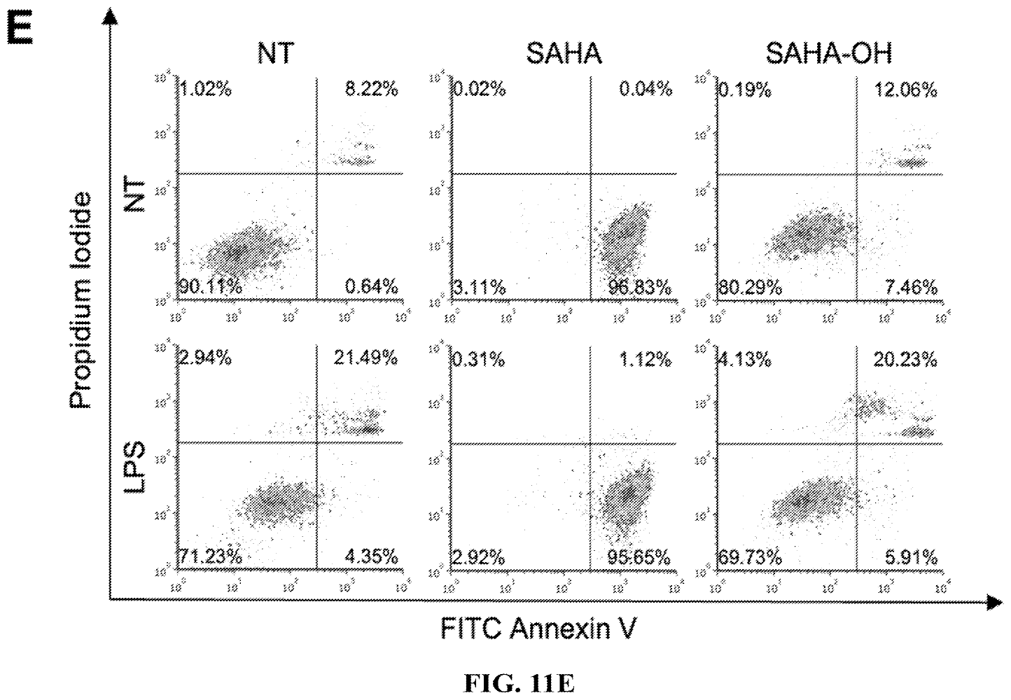

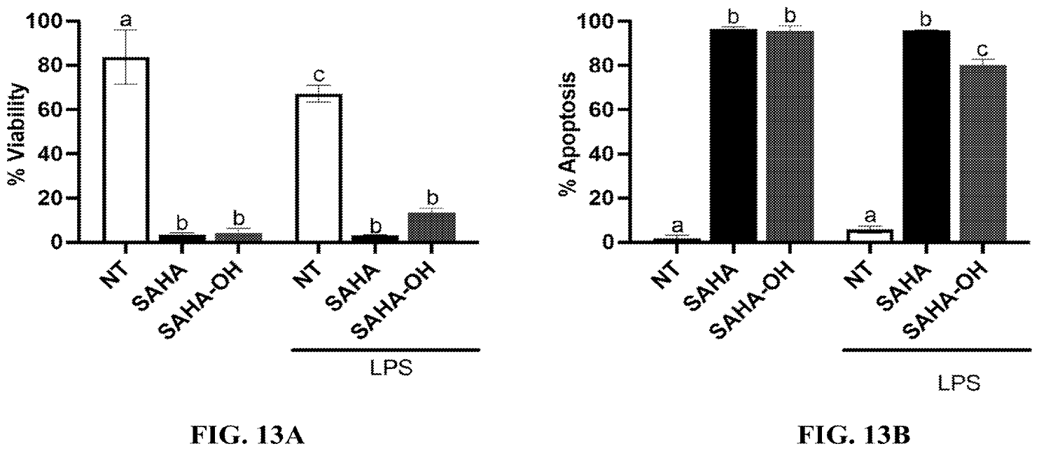

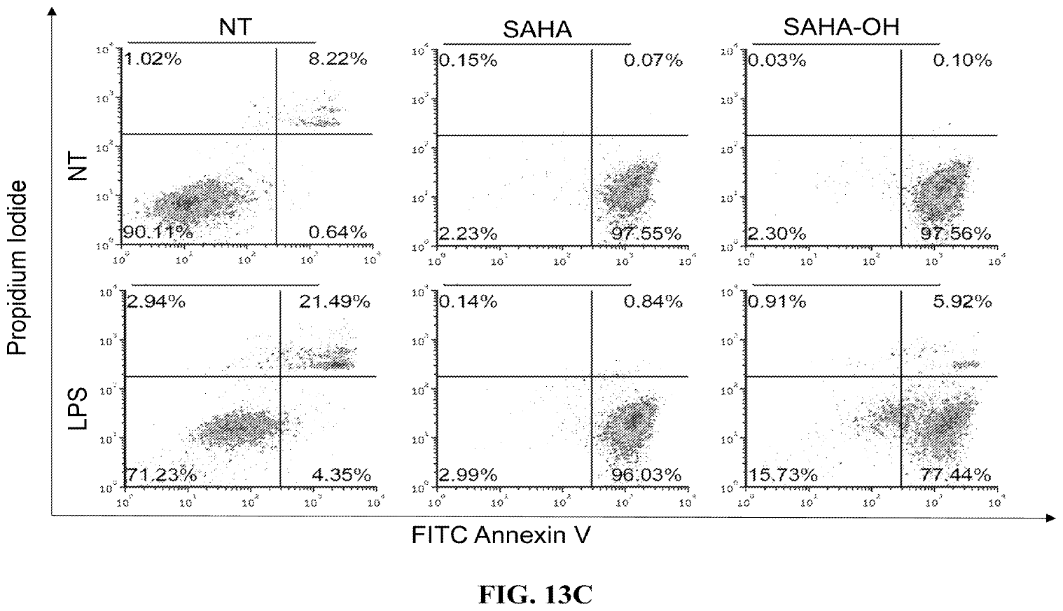

To quantify the differences in toxicity profiles between SAHA and SAHA-OH, flow cytometry was performed using FITC Annexin V (AV) and propidium iodide (PI) staining. FITC-AV and PI staining allowed for the identification of viable cells, apoptotic cells, and dead cells (FIGS. 11A-11E, 12A-12G, and 13A-13C). BMMØs were treated with SAHA and SAHA-OH (0-30 μM) for 3 hours in the presence or absence of LPS stimulation for 48 hours. SAHA and SAHA-OH treated BMMØs elicited a concentration-dependent viability reduction (FIG. 11A) and apoptosis induction (FIG. 11B). The most striking differences between the groups were observed at 10 μM. Treatment with 10 μM SAHA resulted in cellular viability of 3.1% (FIG. 11C) and almost complete induction of apoptosis (96.8%) (FIG. 11D). Conversely, SAHA-OH treatment led to significant improvements in cellular viability through the reduction of apoptosis. No remarkable differences were observed between LPS or no LPS stimulation groups. SAHA-OH treatment resulted in a 24- to 26-fold increase in cellular viability (not significant from NT control) as compared to SAHA treatment, in the absence or presence of LPS stimulation. Representative flow cytometry plots depict the population of SAHA-induced apoptotic cells that are AV⁺/PI⁻ while SAHA-OH-treated cells remained viable (AV⁻/PI⁻) (FIG. 11E).

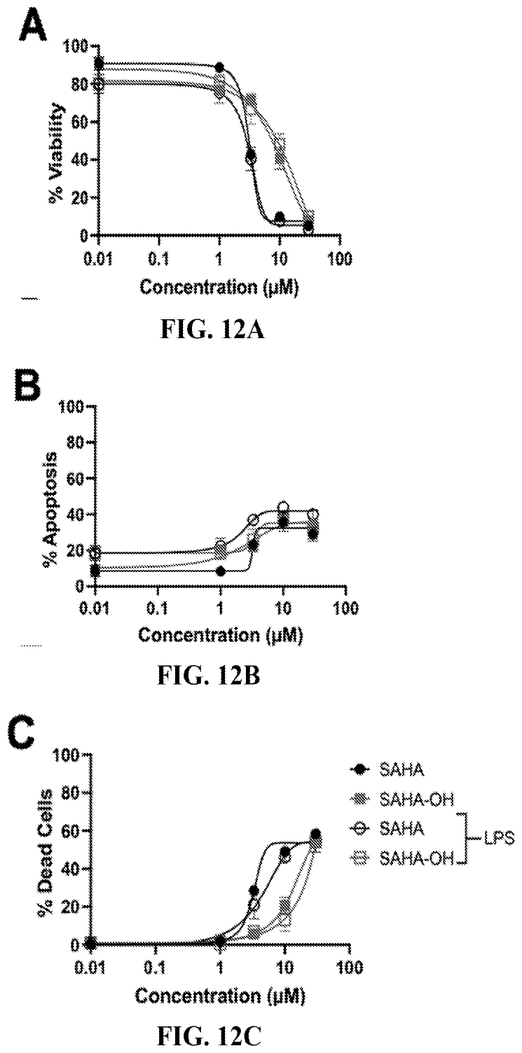

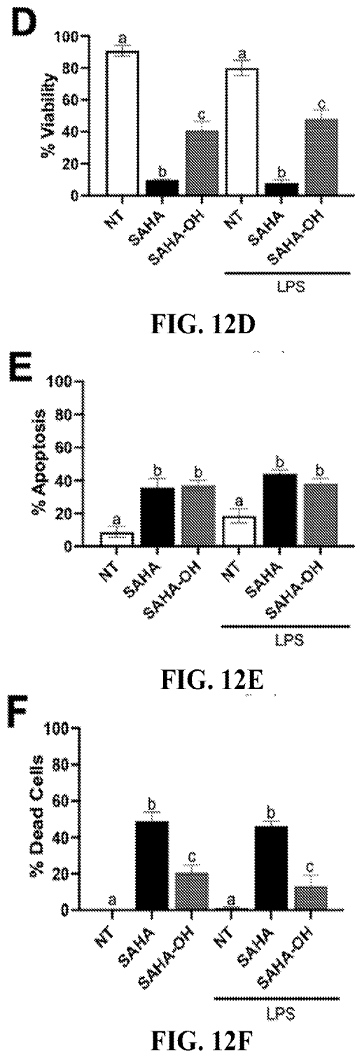

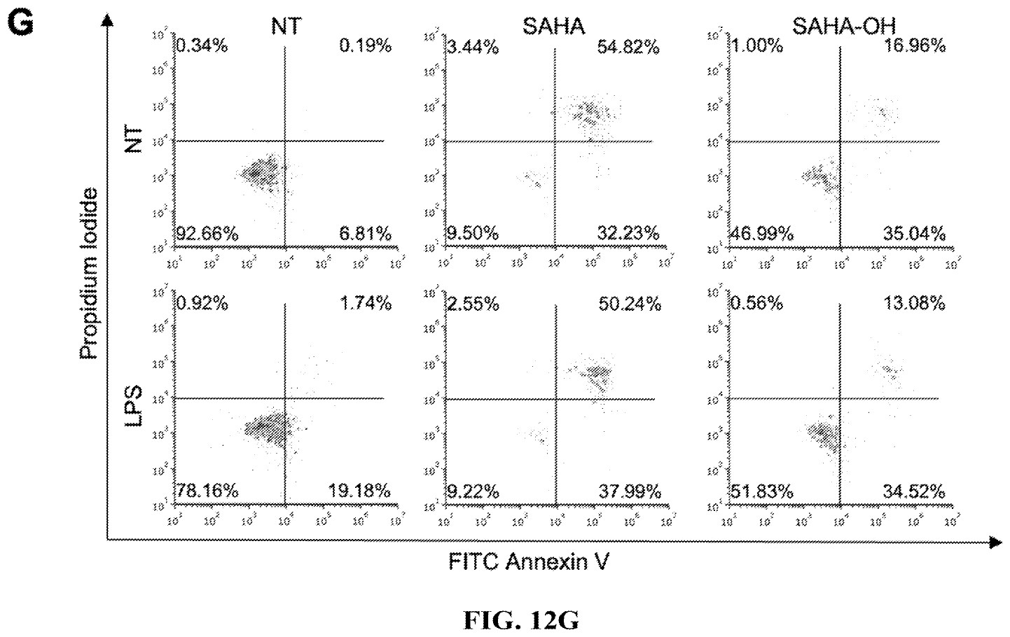

The toxicity profiles for SAHA-OH and SAHA in primary splenic B and T cells was next investigated. Whole splenocytes were isolated and cultured for 3 hours with varying concentrations of SAHA or SAHA-OH (0-30 μM) in the presence or absence of LPS for 24 hours. B cells were identified as B220⁺ and T cells as CD3⁺ using flow cytometry. SAHA and SAHA-OH displayed concentration-dependent toxicity profiles for the B cell population (Figure FIGS. 12A-12C). Similar to BMMØs, 10 μM SAHA treatment resulted in a significant reduction of cell viability to 9.5% for B cells (FIG. 12D) and increased cell death (54.8%) (FIG. 12F). However, treatment with SAHA-OH resulted in a 5-fold enhancement in viability and a 3-fold decrease in cell death for the B cell population. No significant differences were observed between non-LPS stimulated and LPS stimulated B cells. Interestingly, SAHA-OH treatment resulted in a similar B cell apoptotic response as SAHA (FIG. 12E) but showed significantly reduced cell death (FIG. 12F) potentially indicating that B cells are more sensitive to SAHA-induced toxicity than macrophages at the same time point (FIGS. 11A-11E). Representative flow cytometry plots depict the population of SAHA-induced dead cells that are AV⁺/PI⁺, while SAHA-OH-treated cells remained viable (AV⁻/PI⁻) (FIG. 12G). For the T cell population (results not shown), minimal toxicity for both SAHA and SAHA-OH was observed for concentrations up to 30 μM demonstrating the T cells were less affected by SAHA-induced toxicity. This concentration-dependent toxicity for BMMØs and B cells could be attributed to the concentration-limiting range of SAHA, where lower concentrations elicit immunosuppressive effects, while elevated concentrations lead to pro-apoptotic responses. Overall, SAHA-OH reduced the SAHA-associated toxicity profile through the reduction in macrophage apoptosis and B cell death with negligible impacts on T cells.

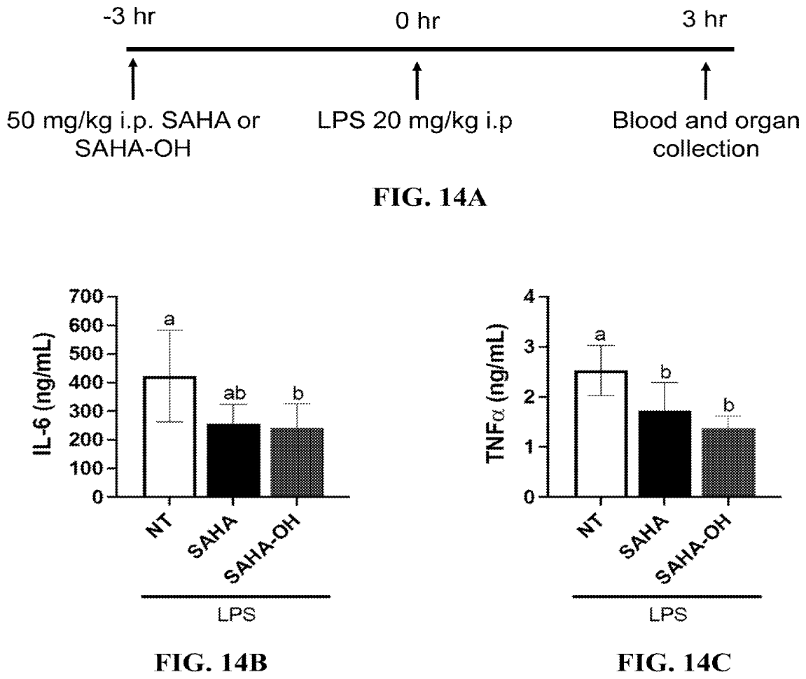

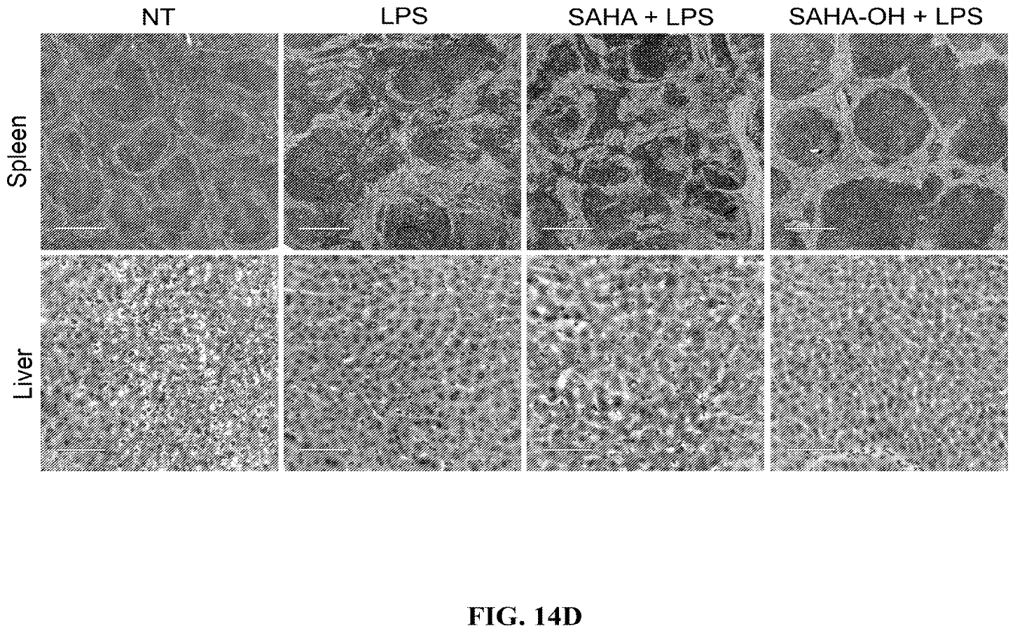

SAHA-OH prevents organ damage while effectively mitigating plasma proinflammatory cytokine levels in an LPS-induced endotoxemia mouse model. To study the biological anti-inflammatory activity of SAHA-OH, an LPS-induced endotoxemia mouse model was employed. SAHA and SAHA-OH were intraperitoneally (i.p.) injected into male C57BL/6 mice 3 hours before the administration of 20 mg/kg LPS i.p. (FIG. 14A). Blood, spleen, and liver samples were collected 3 hours post-LPS challenge and analyzed for cytokines or organ histology. Plasma cytokine measurements of IL-6 and TNFα showed that SAHA and SAHA-OH equally reduced proinflammatory cytokine secretions by about 50% compared to NT control mice (FIGS. 14B-14C). These results demonstrated that the modification of SAHA to SAHA-OH did not negatively affect its anti-inflammatory activity.

To identify potential differences in toxicity, the liver and spleen from mice was isolated and performed hematoxylin and eosin (H&E) staining (FIG. 14D). Samples showed differences in tissue architecture when mice were treated with SAHA or SAHA-OH compared to LPS or NT controls. For NT spleens, highly organized areas of red and white pulp were observed with a well-organized lymphoid follicle. Spleens from LPS-treated controls and SAHA-treated mice exhibited moderate histological disorganization in addition to hyperplastic changes that blurred the boundaries between regions of the white and red pulp. Remarkably, spleens isolated from SAHA-OH treated mice showed similar architecture as NT control and displayed well-organized lymphoid follicles. This further demonstrates the effectiveness of the modified SAHA-OH at attenuating proinflammatory cytokine secretions and reducing splenic organ damage.

### DISCUSSION

In the present study, a novel modification to SAHA at the para position of the phenyl ring is described to produce SAHA-OH that displays improvements in its toxicity profile and in vivo compatibility, without compromising its anti-inflammatory activity. First, SAHA-OH was synthesized and determined to have increased 10- to 47-fold HDAC6 selectivity compared to HDAC1, 2, 3, 8 (FIGS. 5A-5B). The acute toxicity of non-selective pan-HDACi, like SAHA, is thought to be associated with their inhibition of nuclear HDAC1-3. This modest decrease in the selectivity of HDAC1, 2, 3, 8 and reduced nuclear acetyl-histone H3 (FIG. 10) corroborates the improved toxicity profile of SAHA-OH. Compared to other HDAC6 selective inhibitors, like Tubastatin A, SAHA-OH displayed a 1.5-fold less selective HDAC6 inhibition (23 nM compared to 15 nM). On the other hand, SAHA-OH is still approximately 100-fold more selective for HDAC1, 2, 3 compared to Tubastatin A (237-359 nM compared to 30000 nM). Despite the slightly increased HDAC6 selectivity in the modified SAHA-OH compared to SAHA, it reaches the level of selectivity as other classic HDAC6-selective inhibitors. Nonetheless, this modification demonstrated improvements to the current design of SAHA by reducing its pan-selectivity, which may be attributed to the observed protective effects established in this study.

Pan-HDACis have been applied in chronic inflammatory disease models, including rheumatoid arthritis and septic shock, where they are considered anti-inflammatory. The current study confirmed the anti-inflammatory properties of SAHA and demonstrated that SAHA-OH maintained similar anti-inflammatory effects (FIGS. 6A-6C and 8A-8F). Leoni et al. was one of the first groups to demonstrate the anti-inflammatory properties of SAHA through the reduction of LPS-induced proinflammatory cytokine secretion, concanavalin A-induced in vivo hepatotoxicity, along with reduced proinflammatory cytokine secretions in human peripheral blood mononuclear cells. Other groups have also shown that SAHA treatment downregulated genes involved in co-stimulation and cytokine production of BMMØs in response to Toll-like receptor (TLR) agonists. Indeed, the immunomodulatory effects of pan-HDACis are complicated and not merely anti-inflammatory. For example, trichostatin A, a pan-HDACi, has been investigated in multiple models of inflammation with differing results. In one study, inflammatory genes were downregulated in mouse macrophages, in another, proinflammatory genes were upregulated, leading to cytokine secretion. Beyond using pan-HDACis, studies from Vishwakarma et al. demonstrated that the HDAC6 selective inhibitor, Tubastatin A, significantly reduced IL-6 and TNFα secretions in LPS-stimulated THP-1 macrophages and reduced Freund's complete adjuvant-induced animal model of inflammation. Additionally, Tubastatin A has demonstrated improved mice survival in polymicrobial mice models of sepsis. Recent work by Ripamonti et al. established the beneficial use of HDAC6 selective inhibitors in attenuating the dysregulated immune responses seen in severe COVID-19 infections by mitigating the associated cytokine storm responses. These studies attributed these beneficial anti-inflammatory properties to its HDAC6 selectivity. Moreover, the reduced pan-selectivity of SAHA-OH could be linked to its anti-inflammatory properties.

During severe septic shock, global cellular hypoacetylation occurs where SAHA can be utilized to restore acetylation homeostasis. SAHA administration results in the acetylation of histone and non-histone proteins, like histone H3 and α-tubulin, through the inhibition of HDAC enzymes to regulate gene expression. It was ensured that SAHA-OH retained its ability to result in the acetylation of histone H3 and α-tubulin compared to SAHA (FIG. 10). Interestingly, SAHA-OH demonstrated a marginal reduction in histone H3 acetylation, which correlates to the IC₅₀ values showing the reduction in HDAC1, 2, 3, 8 selectivity. Further, SAHA-OH maintained its HDAC6 selectivity, thus effectively resulting in the acetylation of α-tubulin like SAHA. Moreover, the attenuation of histone acetylation has been hypothesized to play a role in the reduced downstream gene transcription related to cytokine expression. The reduced acetylation of H3 histones may also be involved in altering various cellular apoptosis pathways. Therefore, the modification to SAHA allowed for histone and tubulin acetylation; however, the observed reduction in acetylation of nuclear histone H3 may play a role in SAHA-OH anti-inflammatory properties and anti-apoptotic effects.

Dramatic losses of immune cells due to apoptosis results in the propagation of septic complications, ultimately leading to reduced patient survival. SAHA, as an anti-cancer therapeutic, induces apoptosis by triggering both the extrinsic and intrinsic apoptotic pathways through the regulation of various apoptotic genes. The potent antitumor effects of SAHA could also have detrimental effects on host immune cells. Improvements in immune cell viability have shown to result in improved survival in mouse models of sepsis. Therefore, the ability of SAHA to induce apoptosis in primary macrophages was recapitulated; yet beneficially, SAHA-OH did not elicit similar effects at the same concentrations (FIGS. 11A-11E). In addition, lymphocytes, such as B and T cells, often suffer from cell death upon HDACi treatment. In the present study, investigation using ex vivo whole splenocytes demonstrated that SAHA-OH positively improved B cell viability compared to SAHA (FIGS. 12A-12G), yet no observed differences were seen in CD3⁺ T cells. Tubastatin A significantly increased macrophage and B cell populations in a polymicrobial model of sepsis, attributing to the beneficial use of HDACis for improved sepsis outcomes. This observation where the modification to SAHA reduced macrophage apoptosis and B cell death may allow for the widening of the acceptable therapeutic dosing range. This promotes the potential use of SAHA-OH as a sepsis therapeutic, while minimizing potential side effects.

To establish biological relevance, an LPS-induced endotoxemia mouse model serving as a representative model of systemic inflammation was employed. Organ damage is associated with sepsis and severe inflammation, which occurs as a result of excessive cytokine release leading to tissue damage. It was observed that SAHA-OH treatment mitigated LPS-induced cytokine secretions in mouse plasma similarly to SAHA, but strikingly reduced SAHA-associated splenic organ damage (FIGS. 14A-14D). Splenic architecture is often disrupted during severe inflammation as splenocytes undergo hyperplasia followed by lymphoid atrophy. SAHA treatment resulted in disorganized germinal centers within the splenic sections. SAHA-OH effectively reversed the splenic compartment changes induced by SAHA or LPS challenge. Similar to the present work, Zhang et al. demonstrated that pre-treatment with HDACis alleviated acute lung injury induced in a polymicrobial sepsis mice model. This demonstrates the beneficial use of SAHA-OH in reducing acute organ injury in a mouse model of systemic inflammation.

In summary, a modified form of a current FDA-approved HDACi was developed that displays beneficial improvements to its associated toxicity in primary innate and adaptive immune cells as well as in a mouse model of endotoxemia. This data suggests the feasibility of utilizing SAHA-OH as a possible anti-inflammatory therapeutic for the treatment of immune dysregulation observed in severe inflammation and sepsis.

### Example 2. Nanoparticle-Based Immunotherapy Containing Modified Suberoylanilide Hydroxamic Acid Polymer Conjugates to Mitigate Immune Dysregulation in Sepsis

### INTRODUCTION

Suberoylanilide hydroxamic acid (SAHA) was incorporated into immunomodulatory nanoparticles (iNP-SAHA) its effectiveness was examined using a lipopolysaccharide (LPS)-induced endotoxemia mouse model of sepsis. This novel immunotherapy aims to reduce the acute proinflammatory responses and reduce immune cell apoptosis. A pro-drug approach was employed through the covalent modification of poly(lactic-co-glycolic acid) (PLGA) with SAHA-OH to allow for precise incorporation and controlled delivery of SAHA-OH from iNP-SAHA to immune cells and organs. iNP-SAHA delivery led to significant reductions of serum proinflammatory cytokines as well as macrophage apoptosis. Herein, it was demonstrated that iNP-SAHA represents a multi-modal approach which has the potential to treat the multiple dysregulated pathways observed in sepsis.

Suberoylanilide hydroxamic acid (SAHA; vorinostat), a pan-HDACi, has received approval from the Food and Drug Administration for the treatment of cutaneous T cell lymphoma. SAHA induces cell death via the regulation of proapoptotic and anti-apoptotic genes that activates proapoptotic pathways. While beneficial in a tumor environment, this apoptosis-mediated cytotoxicity is also their Achilles' heel, causing growth arrest and apoptosis in healthy immune cells due to lack of targetable delivery. In addition to SAHA's anticancer properties, many studies have established SAHA's anti-inflammatory activity through the suppression of pro-inflammatory cytokine production upon lipopolysaccharide (LPS) challenge in vitro and in vivo. SAHA, as a single-agent therapeutic, is disadvantaged due to its immune cell cytotoxicity, yet possesses beneficial anti-inflammatory capabilities that can be leveraged to mitigate the immune activation seen in sepsis. Strategies in reducing the off-target toxicity of SAHA while promoting its anti-inflammatory properties are needed.

Cargo-less immunomodulatory nanoparticles (iNPs) have been developed that possess the inherent activity to abrogate proinflammatory cytokine secretions induced by LPS by a multimodal mechanism of action including both physical and biological means. It was identified that iNPs did not directly interact with TLR agonists (LPS or CpG ODN) but physically interfered with the interactions between PAMPs and bone marrow-derived macrophages (BMMØs). NF-kB-mediated transactivation in the nucleus is pivotal downstream of various inflammatory stimuli. Then, the composition-dependent abrogation of NF-κB p65 and IkB phosphorylation was assessed. Commercially available polystyrene (PS) or poly(methyl methacrylate) (PMMA) nanoparticles did not reduce LPS-induced NF-κB p65 activation. These studies confirm the multimodal mechanisms by which iNPs function to induce anti-inflammatory immune responses and reinforces their potential to serve as a broad-acting therapeutic for inflammation. Since iNPs do not contain therapeutic ligands, it provides an opportunity to incorporate conjugated biomaterials to permit controlled drug loading to subside potential toxicity of the therapeutic molecule.

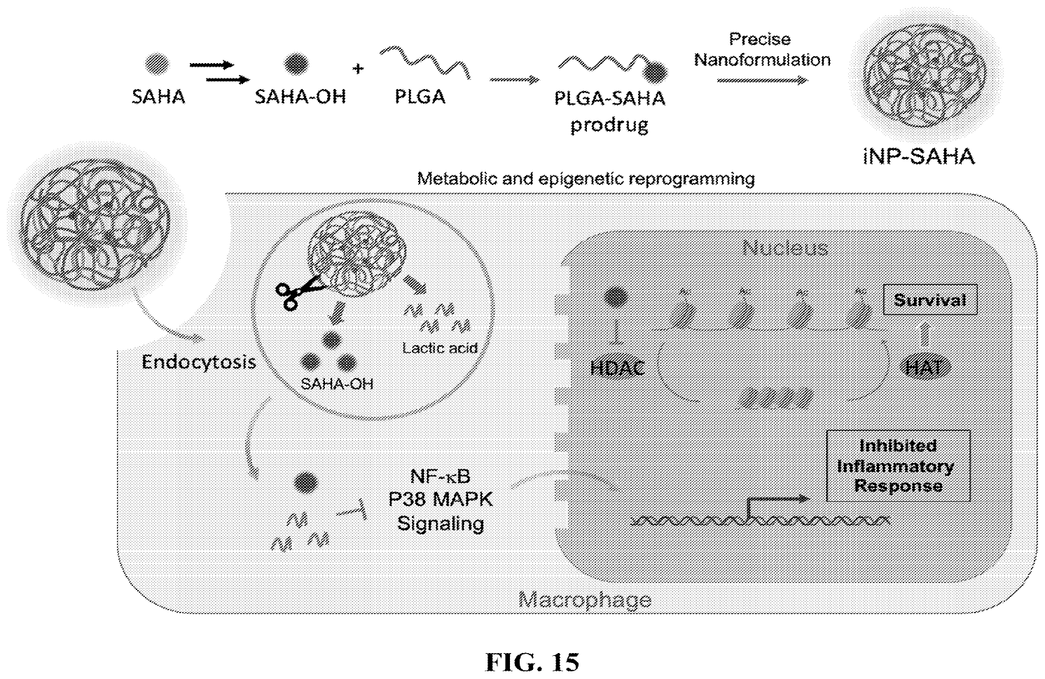

In this study, SAHA incorporated iNPs (iNP-SAHA) were prepared using a prodrug conjugation approach to promote synergistic suppression of proinflammatory responses while mitigating potential drug toxicity (FIG. 15). The combinatorial anti-inflammatory effects of SAHA and iNPs provide a strong approach to attenuate the acute immune activation seen in sepsis. Utilization of nanoparticles incorporating SAHA will promote targeted drug delivery and require lower therapeutic doses to elicit similar effects. It is anticipated that this multi-modal drug delivery approach has the potential to overcome the biphasic immune responses observed in severe inflammation and sepsis to improve patient recovery and address long-term complications.

### Materials and Methods

### Materials

Acid-terminated poly(DL-lactide) (PLA) of low inherent viscosity in hexafluoro-2-propanol ˜0.21 dL/g (approx. 11,300 g/mol) and acid-terminated poly(D,L-lactide-co-glycolide) (PLGA), of low inherent viscosity in hexafuoro-2-propanol ˜0.17 dL/g (approx. 4,200 g/mol) were purchased from Lactel Absorbable Polymers (Birmingham, AL). Poly(ethylene-alt-maleic anhydride) (PEMA) (MW 400,000 g/mol) was purchased from Polysciences, Inc. (Warrington, PA). LPS from Escherichia Coli serotype O111:B4 (Cat #L2630) and RPMI 1640 supplemented with L-glutamine (Cat #R8758) was obtained from Sigma-Aldrich (St. Louis, MO). Penicillin-streptomycin, Versene, and NuPAGE™ 12% Bis-Tris 1.0 mm Mini Protein Gel (Cat #NPO343BOX) were purchased from Thermo Fisher Scientific (Waltham, MA). Fetal bovine serum (FBS) was purchased from VWR (Radnor, PA). L929 cells were purchased from ATCC (Manassas, VA). Acetyl-α-Tubulin (Lys40) (D20G3) Rabbit mAb (#5335), α-Tubulin (11H10) Rabbit mAb (#2125), Acetyl-histone H3 (Lys 9/Lys 14) (#9677) Rabbit mAb, Histone H3 (D1H2) (#4499) Rabbit mAb, and β-Actin (D6A8) (#8457) were purchased from Cell Signaling Technology (Danvers, MA). 2×SDS/PAGE sample buffer was produced using 4% SDS, 5.7 M P-mercaptoethanol, 0.2 M Tris/HCl, pH 6.8, 20% glycerol, and 5 mM EDTA. All chemical supplies were of analytical grade and obtained from Sigma-Aldrich (St. Louis, MO) and CombiBlocks (San Diego, CA) if not mentioned otherwise.

### PLGA-SAHA Conjugation Reaction

SAHA-OH was first conjugated to acid-terminated PLGA by an EDC/NHS carbodiimide chemistry reaction. First, PLGA was dissolved at 20 mg/mL in DMSO and magnetically stirred at 25° C. SAHA-OH, as previously synthesized, was dissolved at 10 mg/mL in DMSO and triethylamine (TEA) (Sigma-Aldrich, St. Louis, MO) was added at 5× excess molar ratio to SAHA-OH. N-(3-dimethylaminopropyl)-N′-ethylcarbodiimide hydrochloride (EDC) (Sigma-Aldrich, St. Louis, MO) crosslinker was dissolved at 20 mg/mL in DMSO and added dropwise to the stirring PLGA solution for 5 minutes. N-hydroxysuccinimide (NHS) (Thermo Fisher Scientific, Waltham, MA) was dissolved at 5 mg/mL in DMSO and added dropwise to the EDC-activated PLGA solution for 10 minutes. SAHA-OH with TEA was added dropwise to the EDC/NHS-activated PLGA solution and stirred overnight to allow the reaction to progress. The resulting PLGA-SAHA conjugate was purified through dialysis utilizing a 3,500 molecular weight cut-off membrane (Thermo Fisher Scientific, Waltham, MA). The conjugate was dialyzed against two DMSO exchanges (500 mL) over a course of 6 hours. To exchange the solvent to water, a series of six distilled water exchanges (4 liters) over 2 days occurred. The conjugates were then frozen at −80° C. for at least 2 hours prior to lyophilization using a Freezone 4.5 μL −50° C. Complete Freeze Dryer System (Labconco, Missouri, USA) for 2 days. The coupling efficiency was calculated through ¹H NMR. ¹H NMR (400 MHz, DMSO-d₆) of PLGA: δ (ppm) 5.20 (m, 1H), 4.91 (s, 2H), 1.47 (m, 3H). ¹H NMR (400 MHz, DMSO-d₆) of SAHA-OH: δ (ppm) 7.52 (d, J=8 Hz, 2H), 7.19 (d, J=8.8 Hz, 2H), 4.40 (s, 2H), 2.26 (t, J=7.2 Hz, 2H), 1.92 (t, J=6.8 Hz, 2H), 1.55 (t, J=6.4 Hz, 2H), 1.47 (t, J=6.8 Hz, 2H). ¹H NMR (400 MHz, DMSO-d₆) of PLGA-SAHA: δ (ppm) 7.52 (d, J=8 Hz, 2H), 7.19 (d, J=8.8 Hz, 2H), 5.20 (m, 1H), 4.91 (s, 2H), 4.40 (s, 2H), 2.26 (t, J=7.2 Hz, 2H), 1.92 (t, J=6.8 Hz, 2H), 1.55 (t, J=6.4 Hz, 2H), 1.47 (m, 2H).

### Preparation and Characterization of Nanoparticles

iNP, iNP-SAHA, and iNP-Cy5.5 were prepared by the oil-in-water (o/w) emulsion solvent evaporation (SE) technique using a similar method as described²¹. Briefly, 200 mg of acid-terminated PLA was dissolved in ethyl acetate to a concentration of 80 mg/mL to generate iNP. For iNP-SAHA, SAHA loading was determined from the coupling efficiency via ¹H NMR. Pre-determined amounts of PLGA-SAHA were added to PLA at 50 mg/mL in ethyl acetate to formulate two loadings of SAHA into iNPs: 9.81 μg/mg iNP-SAHA_{Low}, and 62.29 μg/mg iNP-SAHA_{High}. Cyanine 5.5 (Lumiprobe, Cockeysville, MD) was first conjugated to PLGA (PLGA-Cy5.5) via EDC/NHS carbodiimide chemistry, similarly to the PLGA-SAHA reaction. PLGA-Cy5.5 was added at 0.5% (w/w) to PLA at 50 mg/mL. iNP, iNP-SAHA, and iNP-Cy5.5 synthesis was similarly performed, where 20 mL of 1% PEMA was added and sonicated at 100% amplitude for 30 seconds using a Cole-Parmer 500-Watt Ultrasonic Homogenizer. The resulting o/w emulsion was then added to 80 mL of magnetically stirred 0.5% PEMA overnight until all the ethyl acetate evaporated. The nanoparticles were then collected by centrifugation at 12,000×g for 20 min at 4° C. and washed with 40 mL of Milli Q water. The centrifugation and washing steps were repeated three more times. A mixture of sucrose and mannitol was added to the particle suspension as cryoprotectants to achieve a final concentration of 4% and 3% w/v, respectively. The nanoparticles were then frozen at −80° C. and lyophilized for at least 2 days prior to use. The size and zeta potential of the nanoparticles were determined by dynamic light scattering (DLS) by mixing 10 μL of a 10 mg/mL particle solution into 990 μL of MilliQ water using a Malvern Zetasizer Nano ZSP (Malvern Instruments Inc., Westborough MA) as previously described. For these measurements, iNPs were dispersed in water at pH 6 at room temperature. The Z-average sizes were recorded as the average of at least three measurements.

### Mice

Male and female C57BL/6J (5-7 weeks old) were purchased from The Jackson Laboratories (Bar Harbor, ME). The mice were housed under specific pathogen-free conditions in a facility. All mouse procedures and experiments were compliant to the protocols of the Institutional Animal Care and Use Committee (IACUC).

### Isolation and Generation of Bone Marrow-Derived Macrophages (BMMØs)

BMMØs were generated from isolated bone marrow as previously described.⁴³ Briefly, 5-12-week C57BL/6J female mice were euthanized and the femurs and tibias isolated and flushed with BMMØ media (RPMI 1640 supplemented with L-glutamine (Life Technologies, Carlsbad, CA), penicillin (100 units/mL), streptomycin (100 μg/mL), 10% heat-inactivated fetal bovine serum (FBS) (VWR, Radnor, PA), and 20% L929 (ATCC, Manassas, VA) cell-conditioned media) using a 1 mL syringe and a 25-gauge needle. Once isolated, the cells were pipetted and filtered through a 40 μm cell strainer then plated in uncoated 10 cm non-tissue culture treated petri dishes. The cells were incubated at 37° C. at 5% CO₂ and the media was replaced on days 0, 3, 6, and 8. BMMØs were used for experiments between days 8-10 and were lifted using Versene (Thermo Fisher Scientific, Waltham, MA). Cell number and viability was determined using trypan blue solution and EVE™ Automated Cell Counter (NanoEntek, Waltham, MA).

### Western Blot Analysis

Day 8 BMMØs were seeded at 1×10⁶ cells/well in sterile 6-well plates incubated at 37° C. and 5% CO₂ overnight to allow for cell adherence. BMMØs were treated with 10 μM of SAHA, M of SAHA-OH, 10 μM of PLGA-SAHA, 300 μg/mL iNP, 300 μg/mL iNP-SAHA_{Low}, or 300 μg/mL iNP-SAHA_{High} and incubated for 4, 9, 27, or 48 hours. Cells were lysed with Halt™ Protease Inhibitor Cocktail (Cat #78429) (Thermo Fisher, Waltham MA) in RIPA Buffer (Cat #R0278) (Sigma-Aldrich, St. Louis, MO) and scraped with a cell scraper (VWR, Radnor, PA). Lysates were sonicated with a ⅛th needle tip using a Cole-Parmer 500-Watt Ultrasonic Homogenizer at 40% amplitude for 10 seconds on ice. Lysates were then centrifuged at 4° C. for 20 minutes at 12,000×g. The supernatant was collected and frozen at −80° C. until ready for western blot analysis. Protein lysates were produced using 50/50 sample to 2×SDS/PAGE sample buffer. Proteins were then separated by SDS/PAGE and immunoblotted using Histone H3 (D1H2) XP, Acetyl-Histone H3 (Lys9/Lys14), α-Tubulin (11H10), Acetyl-α-Tubulin (Lys40) (D20G3) XP and β-Actin as primary antibodies. ECL was used for detection.

Immunocytochemistry staining of BMMØs

To examine the uptake and cellular trafficking of iNP to immune cells, day 8 BMMØs were seeded at 0.5×10⁵ cells/well in complete BMMØ media in sterile 8 well chamber slides incubated at 37° C. and 5% CO₂ overnight to allow for cell adherence. The following day, media was replaced with fresh complete media supplemented with 300 μg/mL of iNP-SAHA_{Low}-Cy5.5 or iNP-SAHA_{High}-Cy5.5 formulations. Three hours later, excess iNP-SAHA was removed by washing twice with PBS followed by replacing with complete RPMI 1640 medium containing 100 ng/mL LPS. After 48 hours, cells were fixed with fixation buffer (Biolegend, San Diego, CA) and blocked with blocking buffer (CST, Danvers MA) (#12411) per manufacturer's instructions. Cells were then stained with rabbit acetyl-histone H3 (K9K14) (#9677S) overnight at 4° C. The next day, cells were rinsed with PBS and stained with goat anti-rabbit IgG (H+L), F(ab′)2 Fragment (Alexa Fluor® 488 Conjugate) (#4412) for 1 hour. NucBlue Live ReadyProbes Reagent (Hoechst 33342) (#R37605) was then added to each well and incubated for 15 minutes. Fluoromount-G Mounting Medium with DAPI (#00-4959-52) was added to the top of the chamber slides to seal the coverslip and cured overnight at room temperature. The cells were then imaged using the Nikon Eclipse Ti-2 confocal microscopy (Tokyo, Japan) within 2 days.

### Nanostring Gene Expression Assay

Day 8 BMMØs were seeded at 3×10⁶ cells/well in sterile 6-well plates incubated at 37° C. and 5% CO₂ overnight to allow for cell adherence. BMMØs were treated with 300 μg/mL iNP or 300 μg/mL iNP-SAHA_{High} and incubated for 3 hours. Excess NPs were removed by washing twice with PBS followed by replacing with complete RPMI 1640 medium containing 100 ng/mL LPS. Three hours later, cells were collected and isolated for their RNA using the RNeasy Mini Kit following manufacturer's instructions (Qiagen, Hilden, Germany). The purified RNA was quantified via Nanodrop One (Fisher Scientific, Hampton, NH) and samples were sent to the Institute for Genome Sciences (IGS) Genomics Resource Center (University of Maryland, Baltimore, MD). Nanostring analysis was performed by the IGS core using the nCounter XT CodeSet [XT_PGX_MmV2_Inflammation](#115000082) (NanoString Technologies, Seattle, WA).

### Cytokine Secretion by BMMØs Following LPS Stimulation

To evaluate BMMØ cytokine secretion, day 8 BMMØs were seeded at 1×10⁵ cells/well in complete BMMØ media in sterile 24-well plates incubated at 37° C. and 5% CO₂ overnight to allow for cell adherence. The following day, media was replaced with fresh complete media supplemented with 300 μg/mL of the different iNP or iNP-SAHA formulations. Three hours later, excess iNP or iNP-SAHA were removed by washing twice with PBS followed by replacing with complete RPMI 1640 medium containing 100 ng/mL LPS. After 48 hours, cell culture supernatants were collected and analyzed by enzyme-linked immunosorbent assays (ELISA) (BioLegend, San Diego, CA) to measure murine IL-6 and TNFα. MAGPIX Luminex bead-based multiplex ELISA (Thermo Fisher Scientific, Waltham, MA) measured for 7 various cytokines and chemokines, and data were analyzed using the Luminex xPONENT software (Milli-pore) as per manufacturer's instructions. The 7-plex panel included murine IFNβ, IL-10, TL-6, IL-1β, MCP-1 (CCL2), TNFα, and GROα (CXCL1).

### BAD10 FITC Annexin V (AV) Apoptosis Detection Via Flow Cytometry

Day 8 BMMØs were seeded at 1×10⁵ cells/well in complete BMMØ media in sterile 24-well plates incubated at 37° C. and 5% CO₂ overnight to allow for cell adherence. The following day, media was replaced with fresh BMMØ complete media supplemented with SAHA or SAHA-OH (0-30 M) or 300 μg/mL iNP, 300 μg/mL iNP-SAHA_{Low}, or 300 μg/mL iNP-SAHA_{High}. Three hours later, excess iNP or iNP-SAHA were removed by washing twice with PBS followed by replacing with complete RPMI 1640 medium containing 100 ng/mL LPS. After 48 hours, cells were lifted using Versene and washed with cold PBS supplemented with 1% FBS and 0.4% 0.5 M EDTA (Quality Biological, Gaithersburg, MD). FcR blocking was performed with purified anti-mouse CD16/32 antibody (#101301) (Biolegend, San Diego, CA) prior to staining with Annexin V-FITC and propidium iodide kit (#640914) (BioLegend, San Diego, CA) for apoptosis and cell death. Cell staining was conducted according to BioLegend protocols for flow cytometry. Flow cytometry data were collected using a Becton Dickinson LSR II or Becton Dickinson Canto II flow cytometer. Flow plots and data processing was done using FCS Express 7 Flow Cytometry De Novo Software (De Novo, Glendale, CA).

### Biodistribution

Male C57BL/6 mice (5-7 weeks) were subjected to i.p. injections of either saline, 0.01 mg/mouse PLGA-Cy5.5 conjugates, or 2 mg/mouse iNP-Cy5.5 for 3 hours. Subsequently, mice were subjected to either saline or 20 mg/kg LPS challenge for 3 hours. Various organs were isolated (spleen, kidneys, liver, heart, lung, and GI tract (stomach, pancreas, cecum, and large and small intestines)) and analyzed on the Xenogen in vivo imaging system (IVIS) Spectrum Optical (PerkinElmer, Waltham, MA). Excitation and emission used was 675 nm and 720 nm, respectively.

### Survival

LPS endotoxemia was induced using male C57BL/6J mice (5-7 weeks) (n=6) by intraperitoneal (i.p.) injection with 50 mg/kg SAHA or SAHA-OH or 2 mg of iNP or iNP-SAHA three hours prior to i.p. LPS injection (30 mg/kg). Mice were monitored for a period of 7 days compliant to the protocols set forth by the Animal Care and Use Committee. Mice were euthanized immediately at a humane endpoint noted by acute loss of function and non-sensitivity to touch.

### Cytokine Analysis on the LPS-Induced Endotoxemia Mouse Model

LPS endotoxemia was induced using male C57BL/6J mice (5-7 weeks) (n=6) by intraperitoneal (i.p.) injection with 50 mg/kg SAHA or SAHA-OH or 2 mg of iNP or iNP-SAHA three hours prior to i.p. LPS injection (20 mg/kg). Terminal cardiac blood draws from the left ventricle occurred 3 hours post LPS injections using a 25 g needle and 1 mL syringe into an EDTA coated lavender blood collection tubes (BD Medical, Franklin Lakes, NJ). Blood samples were centrifuged at 1000×g for 10 minutes within 30 minutes of collection. Plasma was extracted and diluted 10,000-fold for ELISA IL-6 measurement and diluted 10-fold for TNFα measurements. MAGPIX Luminex bead-based multiplex ELISA (Thermo Fisher Scientific, Waltham, MA) of mouse plasma (diluted 100-fold) measured for 26 various cytokines and chemokines and data was analyzed using the Luminex xPONENT software (Millipore) as per manufacturer's instructions. The 26-plex panel include murine IL-1β, IL-2, IL-4, IL-5, IL-6, IL-9, IL-10, IL-12p70, IL-13, IL-17A (CTLA-8), IL-18, IL-22, IL-23, IL-27, GM-CSF, IFNγ, TNFα, MCP-1 (CCL2), MIP-1α (CCL3), MIP-10 (CCL4), RANTES (CCL5), MCP-3 (CCL7), Eotaxin (CCL11), GROα (CXCL1), MIP-2a (CXCL2), and IP-10 (CXCL10).

### Hematoxylin and Eosin (H&E) Histological Sectioning

The differences in tissue architecture of liver and spleens were assessed at 3 hours post-LPS administration. Prior to tissue harvest, whole body perfusion was performed by slowly flushing 10 mL of 37° C. PBS through the left ventricle of the heart while a cut in the right atrium allowed for the flow of blood. The liver and spleen were harvested, placed into tissue cassettes, and fixed overnight in 10% buffered formalin. The following day, the organs were paraffin embedded and sectioned in 5 μm slices. Slides were stained for H&E to define histological architecture using standard procedures.

### Statistical Analysis

Statistical analyses were performed using Prism 9 (GraphPad, San Diego, CA). Results are reported as mean±standard deviation (SD). Student's t-test was used to determine the significance of parametric data between two groups. Significant differences between multiple treatment groups were determined by one-way ANOVA along with Tukey's multiple comparison test. Mouse survival was reported using a Kaplan-Meier survival curve and statistical significance of mouse survival was determined with a log-rank (Mantel-Cox) X² test. In all cases unless otherwise noted, p<0.05 was considered to be statistically significant.

### Results

### PLGA-SAHA Conjugate Synthesis and Characterization

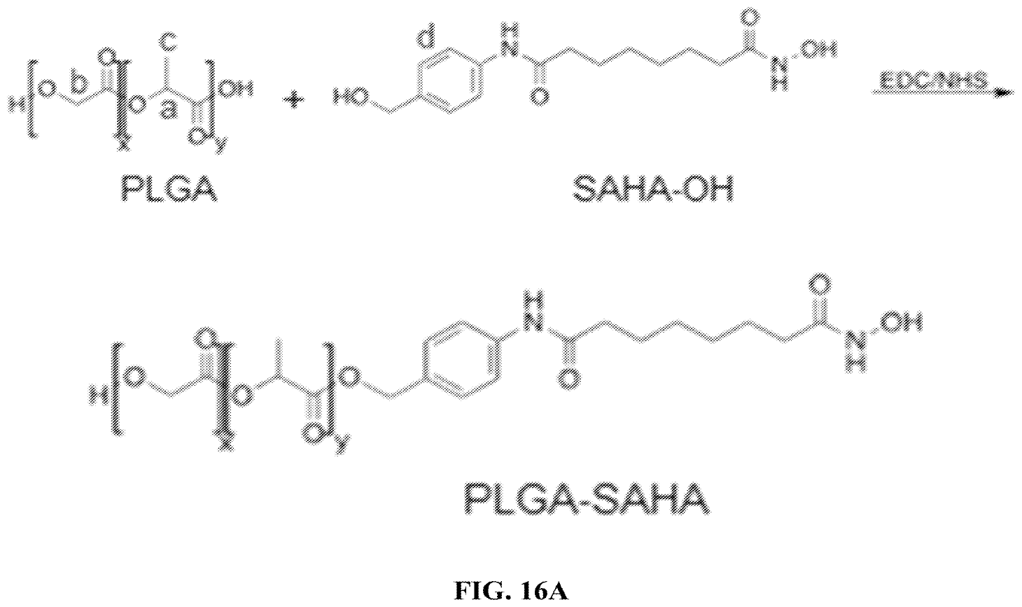

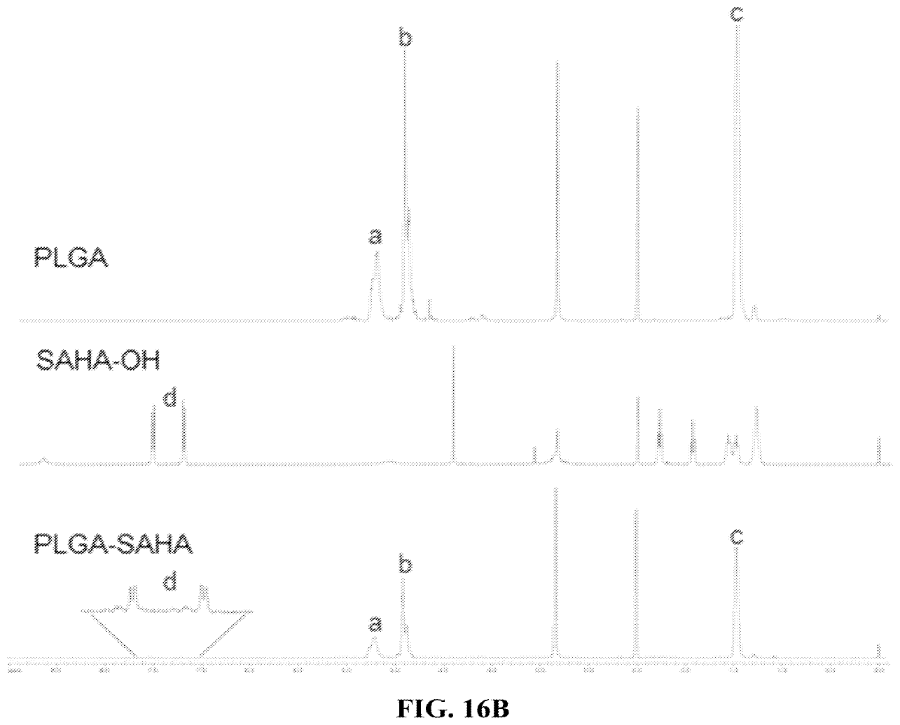

To generate the pro-drug PLGA-SAHA conjugates, SAHA-OH was utilized, which was previously found to show potent anti-inflammatory properties and reduced toxicity compared to SAHA [Truong et al. 2022 submitted], was conjugated to carboxyl-terminated PLGA by an EDC/NHS reaction and confirmed to have a 77.5% coupling efficiency by ¹H-NMR (FIGS. 16A-16B). To examine if the conjugation of SAHA to PLGA maintained its ability to acetylate histones, immunoblotting of SAHA, SAHA-OH, and PLGA-SAHA conjugate-treated BMMØs at two time points (4 and 9 hours) was performed to measure the acetylation (Ac) of α-tubulin and histone H3 (FIG. 10). SAHA-OH showed reduced Ac—H3 activity compared to SAHA; however, their Ac-tubulin activity was similar. PLGA-SAHA treatment resulted in marginal reduction in H3 and tubulin acetylation; however, effectively demonstrated that acetylation occurred as compared to no treatment. This confirmed that the conjugation of PLGA to SAHA-OH did not result in the loss of activity as measured by its acetylation capacity, thus allowing for its incorporation into iNPs.

Formulation of iNP-SAHA and its Trafficking to BMMØs

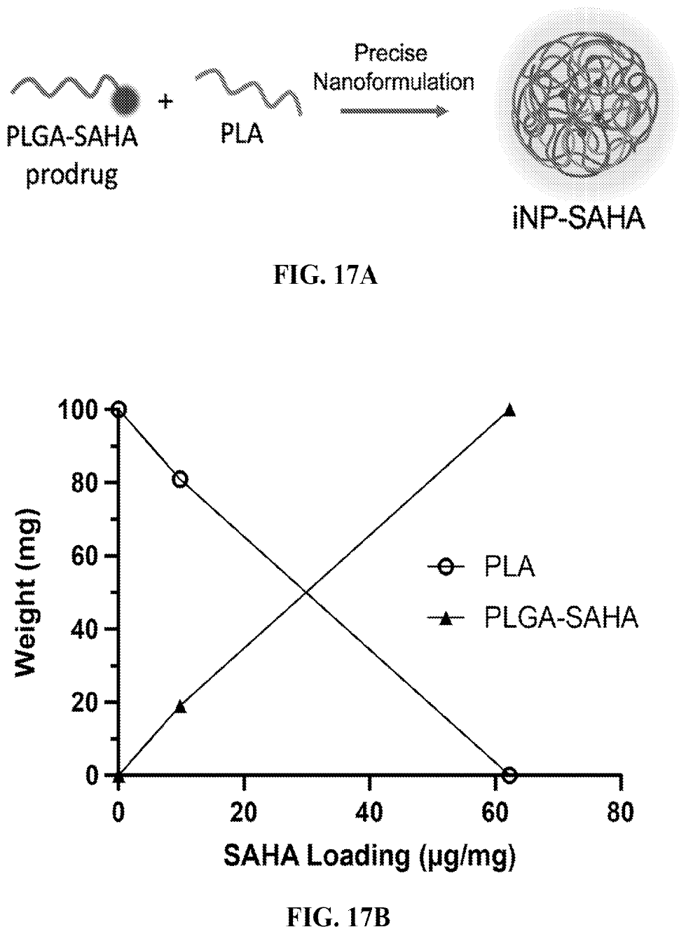

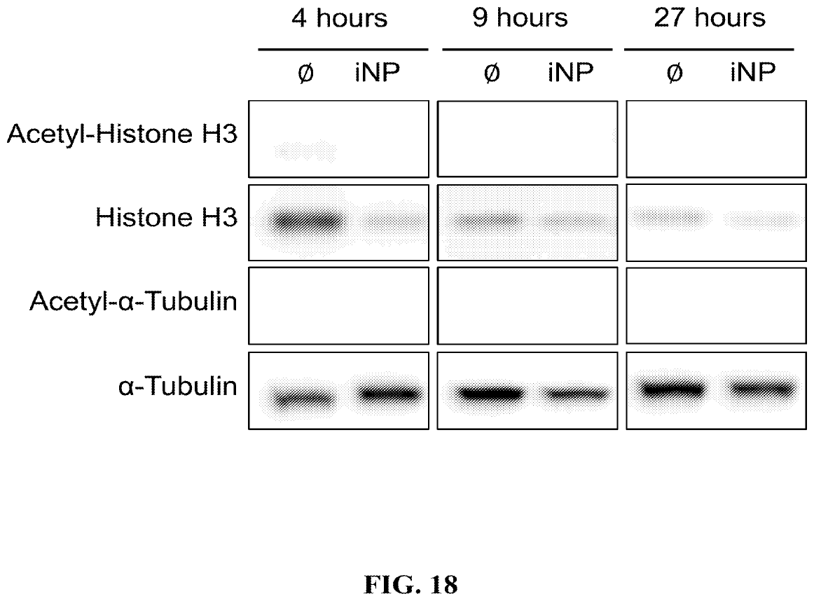

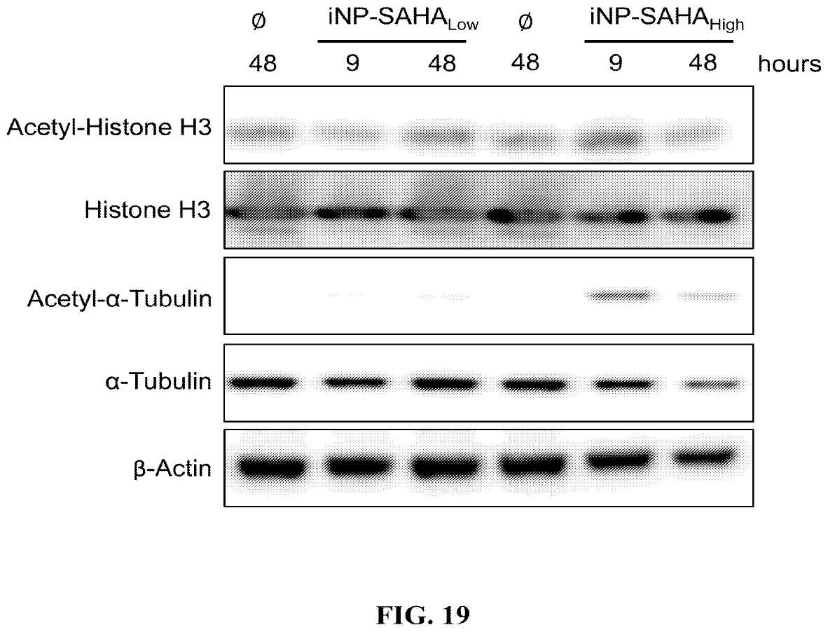

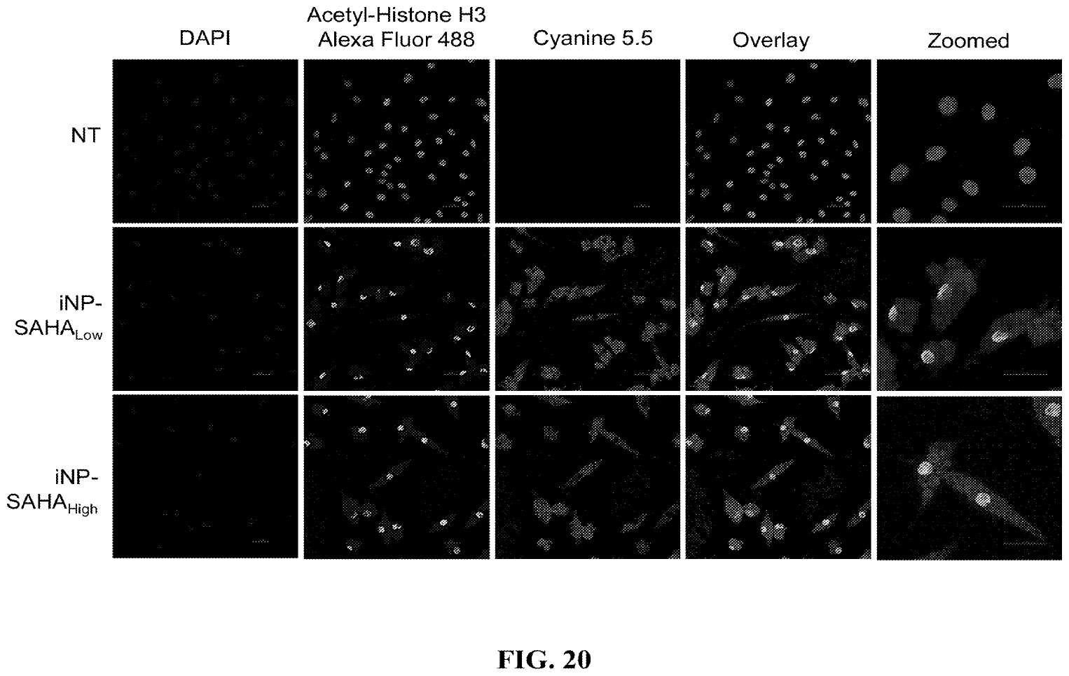

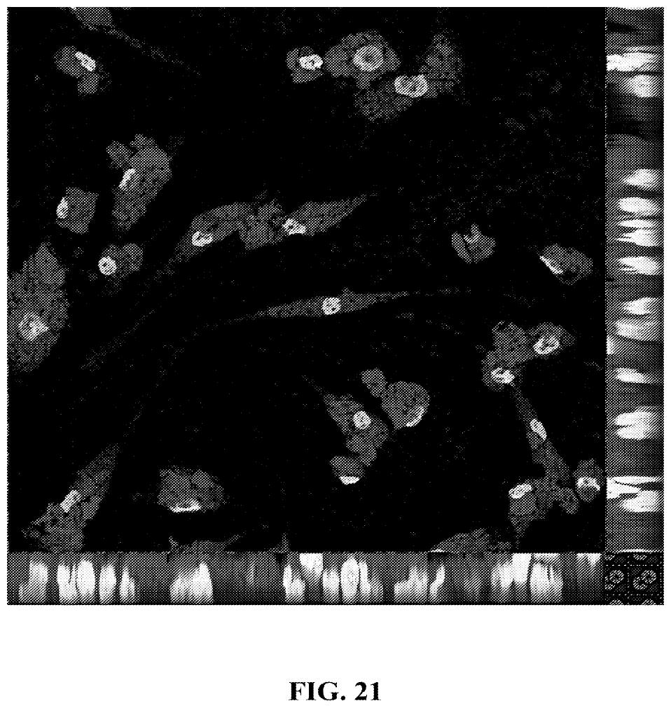

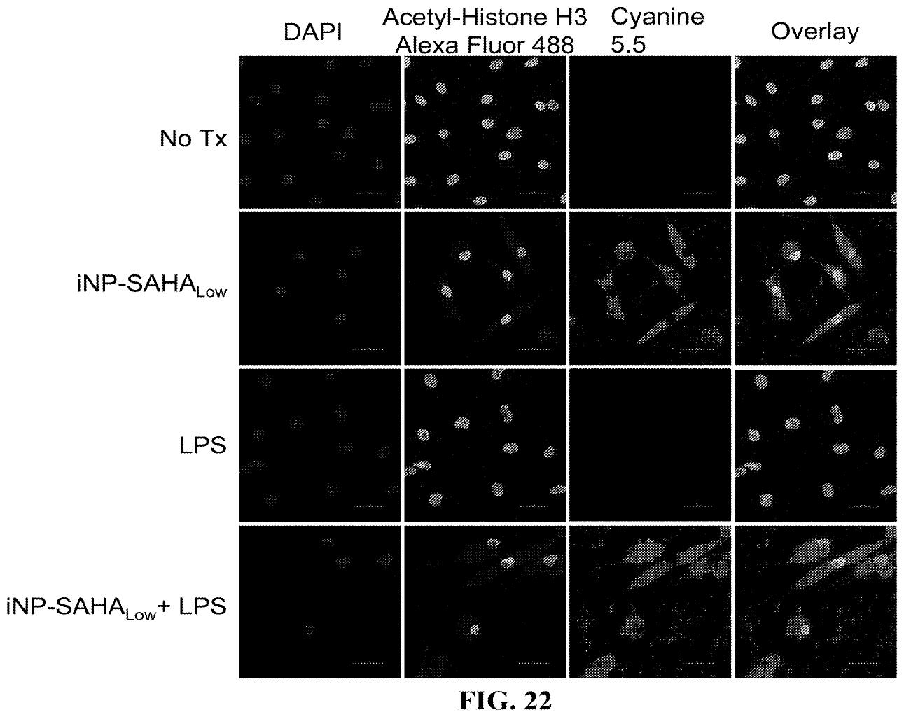

Two different loadings of iNP-SAHAs were prepared by mixing the PLGA-SAHA conjugate with unmodified PLA polymer at precise stoichiometric ratios by single oil-in-water (o/w) emulsion technique (FIGS. 17A-17B). As shown in Table 1, iNP-SAHA were optimized and formulated with precise physicochemical properties ideal for delivery to phagocytic immune cells. Western blotting was used to ensure that iNPs did not modulate histone H3 or α-tubulin acetylation (FIG. 18). Interestingly, iNP-SAHA_{Low} and iNP-SAHA_{High} treatment effectively resulted in the acetylation of histone H3; however, more acetylation was observed in α-tubulin using iNP-SAHA_{High} (FIG. 19). Next, the ability of iNP-SAHA to deliver SAHA through macrophage internalization was investigated via phagocytosis of Cyanine 5.5 (Cy5.5)-labeled iNP-SAHAs (FIG. 20). BMMØs were pre-treated for 3 hours with iNP-SAHA_{Low}-Cy5.5 or iNP-SAHA_{High}-Cy5.5, then excess NPs were washed off and cells were stained and imaged 48 hours later via confocal microscopy. The presence of Cy5.5 within the cells confirms effective iNP internalization, and z-stacking analysis verifies iNPs were internalized rather than coating the exterior of the cell (FIG. 21). BMMØs were also stained for acetyl-histone H3, and iNP-SAHA treatment resulted in increased Alexa Fluor 488 intensity demonstrating increased histone H3 acetylation within the nucleus. Therefore, this shows that SAHA can be delivered by iNP-SAHA and allow for a biological effect. Interestingly, under inflammatory conditions, the uptake of iNP-SAHA was not significantly affected (FIG. 22). Immunocytochemistry staining of BMMØs confirmed the effective iNP-SAHA uptake into phagocytic cells and the retention of SAHA-OH activity following iNP-SAHA formulation and intracellular pro-drug processing, thus limiting potential off-target toxicities and systemic exposure.

TABLE 1
Characterization of iNP and iNP-SAHA through DLS

| | SAHA | | | Zeta |
|---|---|---|---|---|
| | Loading | Size | | potential |
| Type | (μg/mg) | (nm) | PDI | (mV) |
| iNP | 0 | 605.3 ± 9.5 | 0.441 | −45.8 ± 0.46 |
| iNP-SAHA_{Low} | 9.81 | 721.6 ± 10.8 | 0.300 | −48.1 ± 0.25 |
| iNP-SAHA_{High} | 62.29 | 755.3 ± 9.6 | 0.281 | −47.2 ± 0.57 |
| iNP-Cy5.5 | 0 | 591.2 ± 4.9 | 0.197 | −43.6 ± 0.06 |
| iNP-SAHA_{Low}-Cy5.5 | 9.81 | 700.0 ± 40.3 | 0.218 | −44.8 ± 1.27 |
| iNP-SAHA_{High}-Cy5.5 | 55.93 | 528.3 ± 3.8 | 0.206 | −47.5 ± 0.49 |

iNP = cargo-less immunomodulatory nanoparticles containing no SAHA. Two loadings of iNP-SAHA were formulated and labeled as Low and High. Formulations with Cy5.5 were synthesized for imaging studies. DLS characterization determined the particle size (nm), polydispersity index (PDI), and zeta potential (mV). All data is expressed as means ± SD (n = 3).

iNP-SAHA Mitigates Proinflammatory Cytokine Responses and Improves Cellular Viability in BMMØs

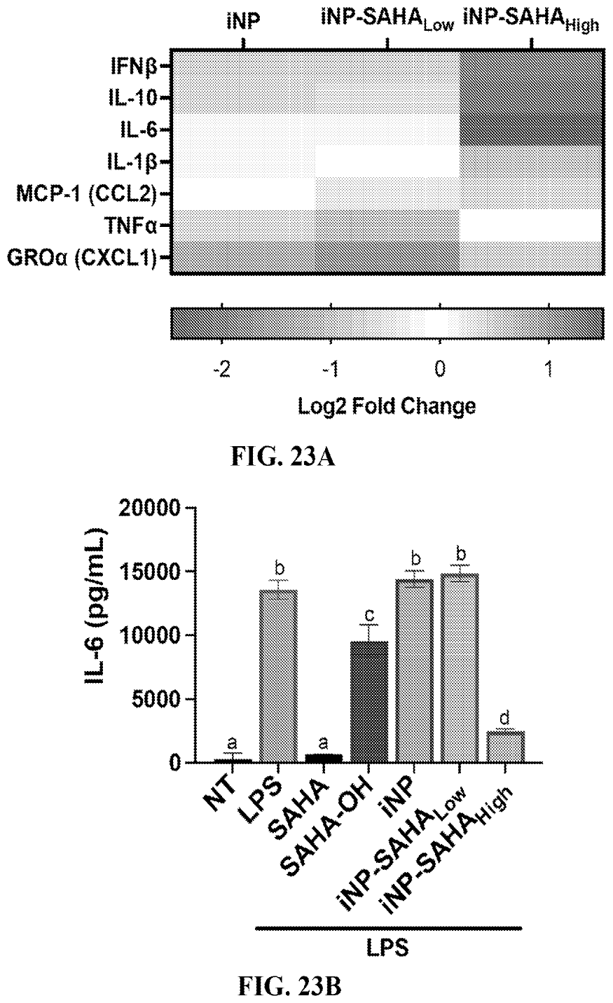

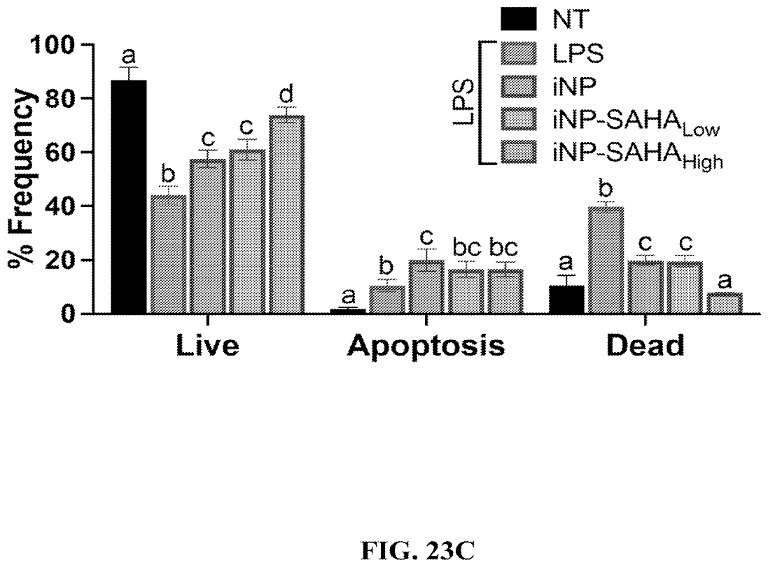

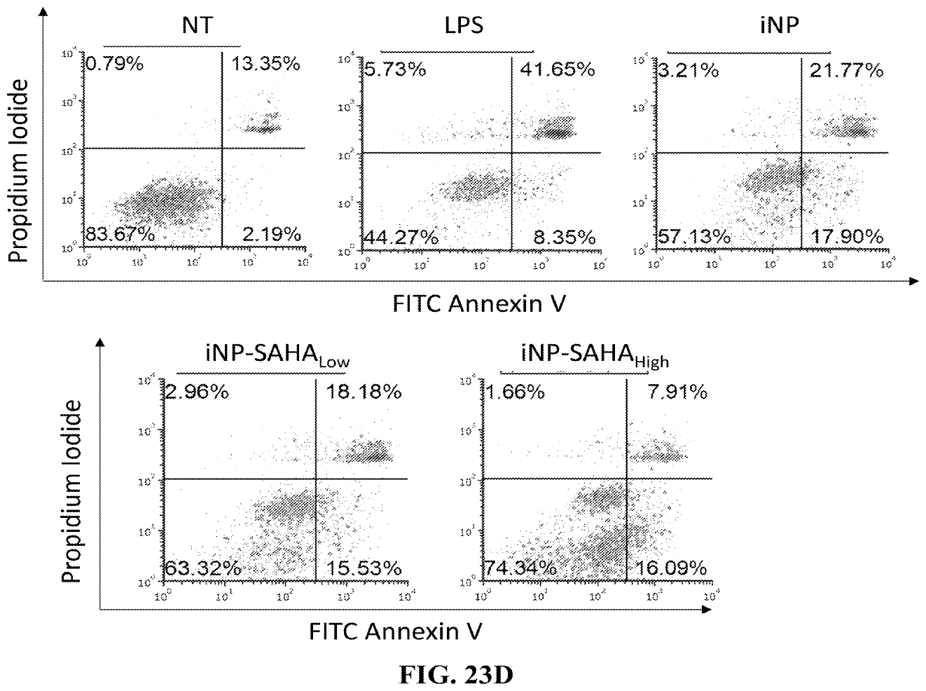

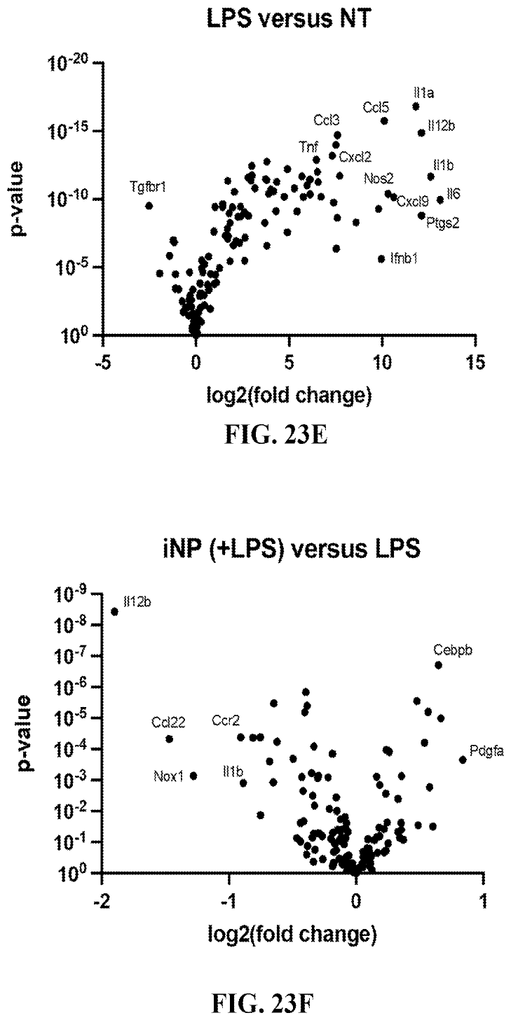

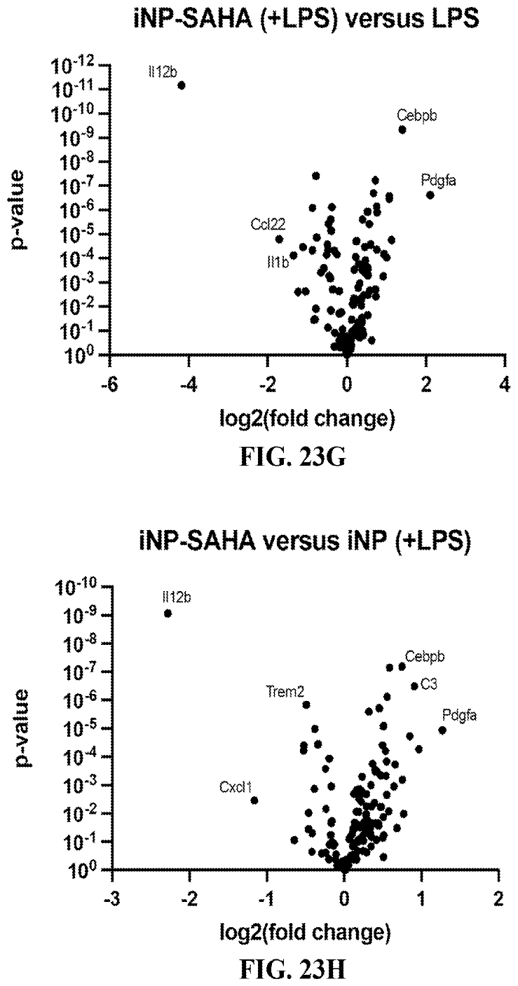

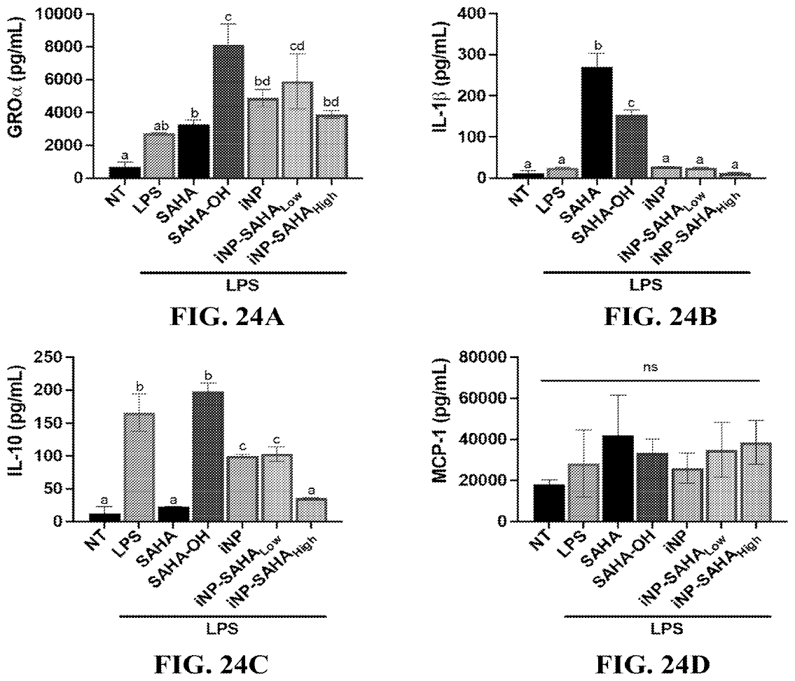

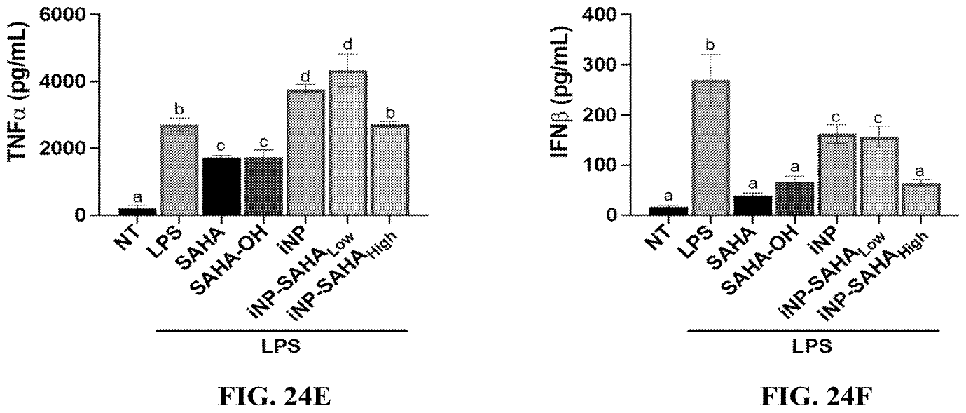

To analyze the in vitro biological function of iNP-SAHA, anti-inflammatory properties were examined through the suppression of proinflammatory cytokine mediators along with demonstrating improved cellular viability of BMMØs upon LPS challenge. All iNP-SAHA and iNP formulations were treated at 300 μg/mL, where this concentration was deemed more effective (data not shown). Indeed, iNP-SAHA_{High} performed significantly better than cargo-less iNPs at suppressing cytokine secretions from BMMØs stimulated with LPS (FIG. 23A). iNP-SAHA_{High} significantly reduced IL-6 secretions as compared to iNP-SAHA_{Low} or iNP, and similarly suppressed IL-6 as compared to soluble SAHA controls (FIG. 23B). To further investigate iNP-SAHA effect on other proinflammatory cytokines and chemokines, a Luminex bead-based immunoassay was used (FIGS. 24A-24F). iNP-SAHA_{High} either significantly reduced or similarly suppressed GROα, IL-1β, IL-10, MCP-1, TNFα, and IFNβ. Furthermore, the results also demonstrated that iNP-SAHA_{High} possessed a favorable improvement in viability and reduction in necrosis, thus furthering its ability to combat the loss of leukocytes through cell death (FIG. 23C). Further, there were no statistical differences in iNP-SAHA_{Low's} ability to prevent apoptosis or necrosis compared to iNPs. Representative flow cytometry plots depict the populations of NT, LPS, iNP, iNP-SAHA_{Low}, and iNP-SAHA_{High}-induced apoptotic cells that are AV+/PI− while SAHA-OH-treated cells remained viable (AV−/PI−) (FIG. 23D).

Future work expanding these findings to address lymphocyte apoptosis using iNP-SAHA will be undertaken, as was similarly performed using SAHA-OH cultured with B and T cells [Truong et al. 2022 submitted]. These findings demonstrated that iNP-SAHA could simultaneously reduce proinflammatory and apoptotic responses in BMMØs induced by LPS, offering a synergistic delivery system with the potential to address the multifaceted dysregulated immune responses seen in sepsis.

iNP-SAHA Modulates Gene Expression Profiles in BMMØs

To understand how iNP-SAHA treatment modulated the gene expression profiles of BMMØs, the NanoString nCounter Mouse Inflammation v2 Panel was employed, which enabled the profiling of 248 inflammation-related mouse genes (FIGS. 23E-23H). The NanoString platform has high reproducibility, sensitivity, and is associated with a low background signal. Volcano plots were used to identify differentially expressed genes (DEG) between various treatment groups comparing the log 2 fold differences. DEGs were identified as genes with at least 2 log 2 fold difference. P values for NanoString data was obtained from nSolver software using a minimum count of 50 as a cutoff. A total of 134 genes met the inclusion criteria. Of these genes, 59 DEGs for LPS vs. NT, 0 DEGs for iNP (+LPS) vs. LPS, 2 DEGs for iNP-SAHA (+LPS) vs. LPS, and 1 DEG for iNP-SAHA (+LPS) vs. iNP (+LPS) were identified. Compared to NT, LPS treatment significantly increased the expression of Il6, Il-1b, Il-12b, Il-1a, Ifnb1 and several others. iNP treatment under LPS stimulation conditions most significantly reduced Il12b expression (3.73-fold) followed by Ccl22 (2.8-fold) compared to LPS treated BMMØs. The reduction of Il12b was more significantly potentiated using iNP-SAHA treatment under LPS stimulation (18.2-fold) compared to LPS treated BMMØs. Ccl22 was also further reduced compared to LPS but only modestly more than iNP groups. Finally, comparing iNP-SAHA and iNP treated BMMØs under LPS stimulation identified significant reductions in Il12b and Cxcl1 expression and Pdgfa was increased. Overall, the most dramatic reductions in gene expression occurred for Il12b, Ccl22, and Cxcl1, whereas Pdgfa and Cebpb were overall increased for the iNP-SAHA treated groups.

iNP-SAHA Distributed to the Liver and Spleen and Offered Protection Against LPS-Induced Endotoxemia

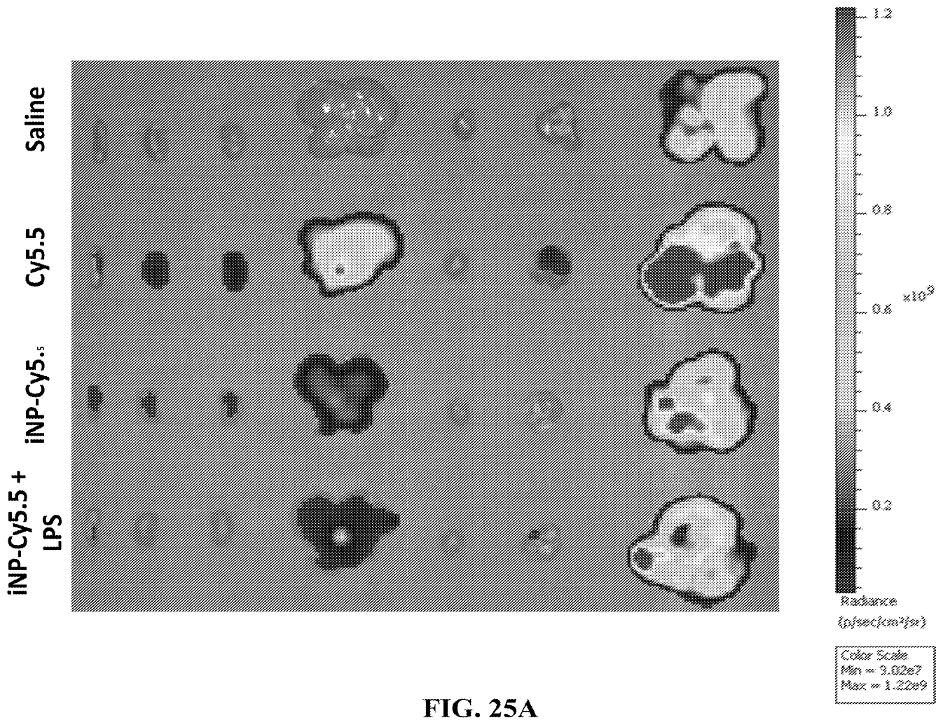

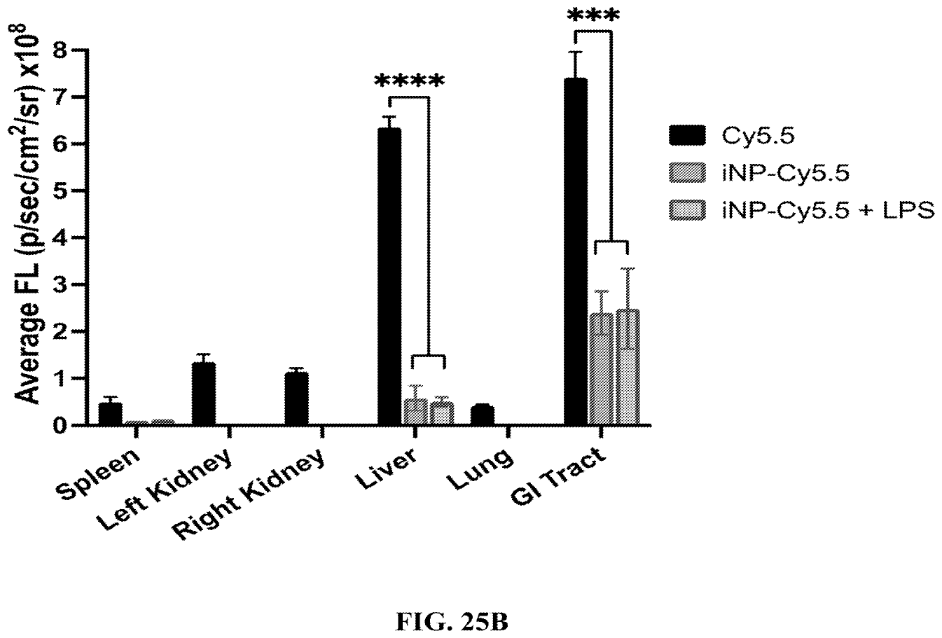

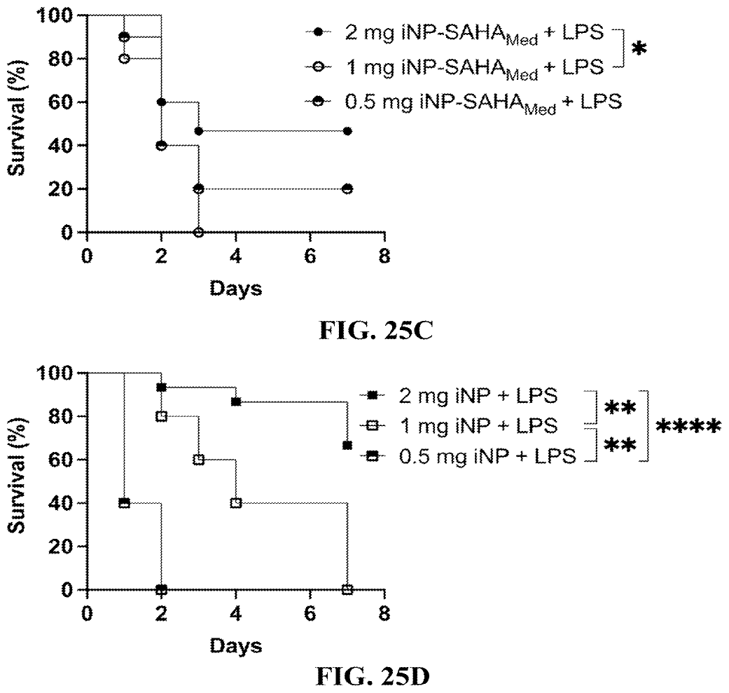

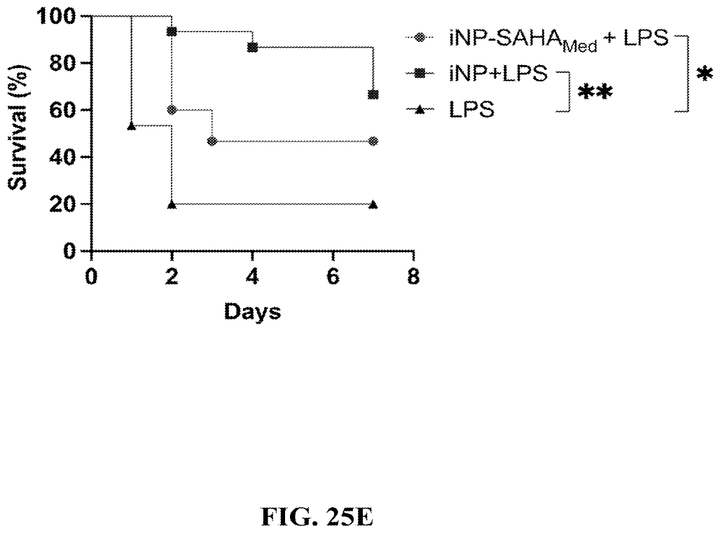

To examine the biodistribution of iNPs in vivo, iNP was formulated to incorporate Cy5.5 to observe for organ distribution. Mice were subjected to intraperitoneal (i.p.) injections of either saline, soluble PLGA-Cy5.5 conjugates (Cy5.5), or iNP containing Cy5.5 (iNP-Cy5.5), then 3 hours later subjected to either saline or LPS challenge (20 mg/kg). Subsequently, various organs (spleen, left and right kidney, liver, heart, lungs, and GI tract) were isolated 3 hours later and imaged using an in vivo imaging system (IVIS) (FIG. 25A). It was observed that soluble Cy5.5 had a larger systemic distribution compared to iNP delivered Cy5.5, as observed with higher fluorescent intensities in the various organs (FIG. 25B). The lowered Cy5.5 fluorescence in the liver, spleen, and kidney, through iNP delivery is not unreasonable, as iNP delivery allows for controlled targeted distribution to specific immune cells, which may result in reduced systemic exposure. Overall, the presented data showed that iNPs can serve as an effective platform for delivery of therapeutic cargos to designated immune cell populations at organ sites like the liver and spleen.

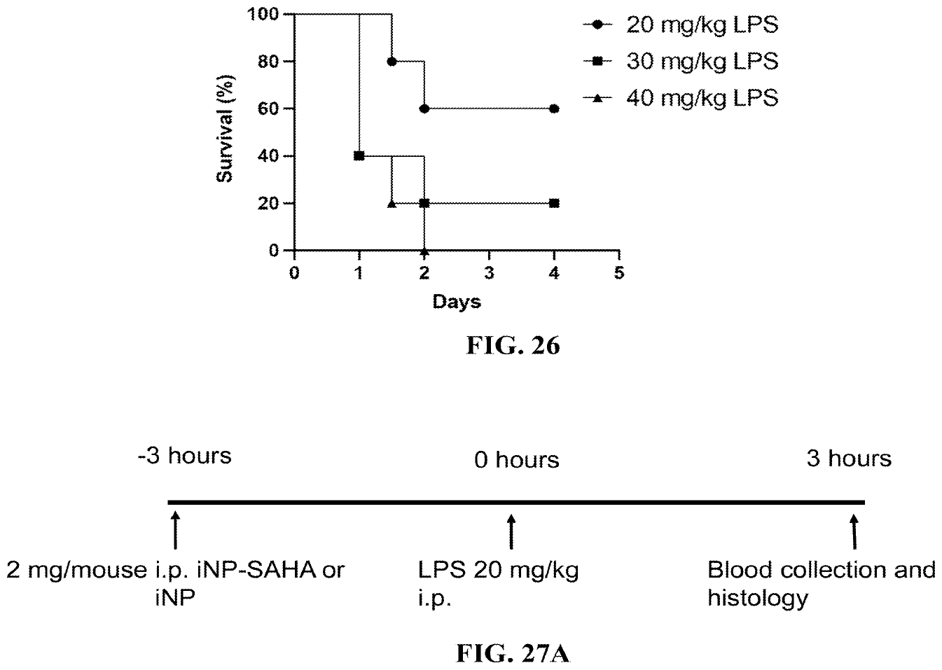

To study the protective effects of iNP-SAHA under LPS-induced endotoxemia, various survival studies were performed. First, a dose escalation study determined that the lethal dose of LPS was 30 mg/kg (FIG. 26). Dose escalation studies focused on varying the nanoparticle concentration determined that higher 2 mg iNP-SAHA (FIG. 25C) or 2 mg iNP (FIG. 25D) were more effective at protecting against LPS-induced sepsis onset. As a result, cross comparison between iNP-SAHA and iNP on improving mouse survival were performed. Briefly, mice were subjected to iNP-SAHA i.p. injections (n=15) or iNP i.p. injections (n=15) 3 hours prior to 30 mg/kg LPS insult. As a control, mice subjected to only LPS were pretreated with saline (n=15). The iNP-SAHA-treated mice had an improved survival of 47% after LPS challenge, while iNP-treated mice had a higher survival rate of 67%, compared to the control LPS-treated mice (20% survival) (FIG. 25E). Interestingly, iNP and iNP-SAHA did not perform significantly different from each other. It has been observed in various studies from Li et. al that the treatment of SAHA can improve overall mouse survival, before and after LPS challenge. This data demonstrated the modular capabilities of iNP-SAHA through the effective biodistribution to lymphoid organs and protection against LPS-induced endotoxemia. It is likely that the improvement in survival results from multiple mechanisms that act in concert to elicit a protective effect. iNPs significantly downregulated the activation of NF-kB and p38 MAPK though its degradation product, lactic acid in a GPR68-dependent manner. Addition of SAHA into iNPs may have further contributed to the downregulation in NF-kB and p38 MAPK.

iNP-SAHA Reduces Plasma Levels of Proinflammatory Cytokines Demonstrates Organ System Biocompatibility

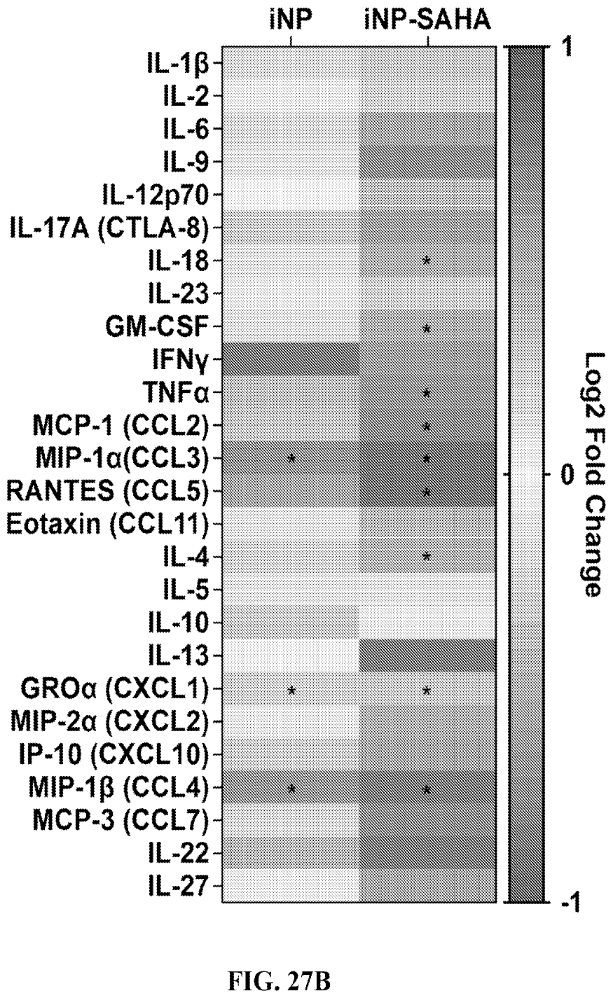

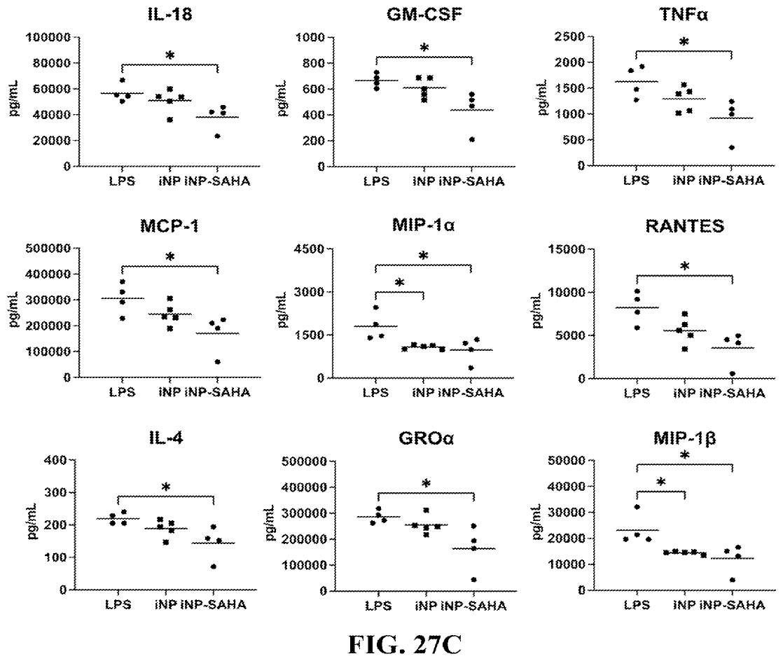

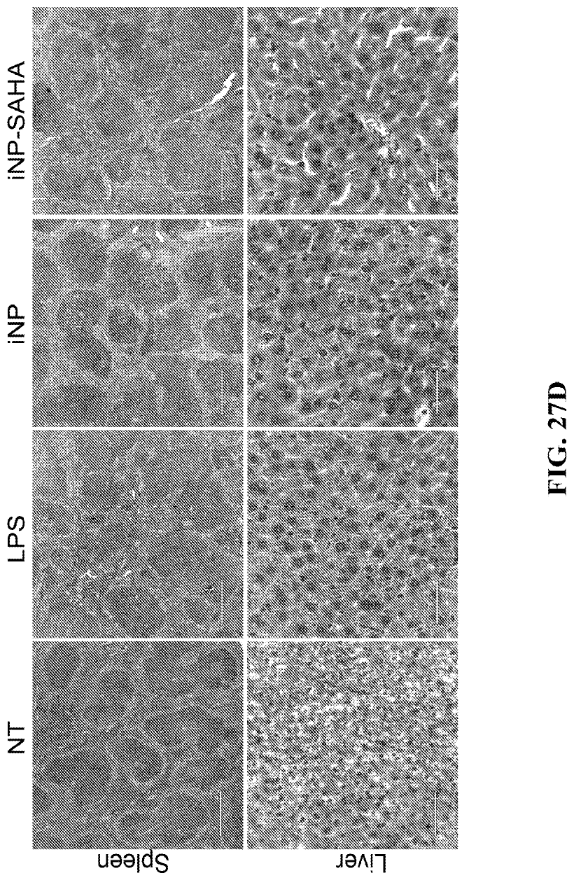

To examine the in vivo efficacy of iNP-SAHA, mice were subjected to a three-hour pre-treatment of iNP or iNP-SAHA i.p. injections then subsequently injected with LPS. LPS control mice were injected with saline 3 hours prior to LPS challenge. After three hours post LPS insult, terminal cardiac blood draws were performed to allowed for plasma collection for further processing (FIG. 27A). Extensive multiplex analysis by Luminex for 26 various cytokines and chemokines was performed to provide systemic insight into the anti- or proinflammatory action of iNP-SAHA, depending on the context of the tissue site along with the cytokine concentration and duration (FIG. 27B). Treatment of iNP-SAHA in vivo significantly reduced serum levels of various cytokines and chemokines, such as IL-18, GM-CSF, TNFα, MCP-1, MIP-1α, RANTES, TL-4, GROα, and MIP-1β, compared to LPS-treated mice (FIG. 27C). These mediators are characteristic markers of sepsis, and their elevated levels can contribute to sepsis progression through the activation of innate immune responses and recruitment of inflammatory immune cells. The suppression of these proinflammatory markers is indicative of iNP-SAHA ability to elicit an anti-inflammatory effect to combat the acute proinflammatory phase of sepsis. To establish biocompatibility and biodistribution of iNP-SAHA in LPS-induced mice, organ histology and in vivo imaging were performed. Spleen and liver histological samples from iNP and iNP-SAHA treated mice showed no remarkable changes in organ architecture compared to controls, demonstrating biocompatibility and lack of toxicity of the treatments (FIG. 27D). Furthermore, it has been shown that loading SAHA into polymeric-based nanoparticles are biocompatible within mice models, along with improving drug delivery and distribution into various organ systems. These findings demonstrate the safety, tolerability, and distribution of iNP-SAHA for subsequent use in LPS-induced endotoxemia mice models.

### Discussion

A multimodal iNP platform was developed through the incorporation of SAHA which has inherent synergistic anti-inflammatory properties and reduced off-target toxicities to address the immune dysregulation seen in severe inflammation and sepsis. There is no FDA-approved treatment option available to improve patient survival and many of these trials have focused on eliminating the increase in proinflammatory cytokines and the corresponding organ damage. It has been hypothesized that the profound clinical heterogeneity of sepsis and treatments only targeted towards a single molecular pathway are major contributors to the diminished clinical trial outcomes. This immunotherapy has the potential capabilities to target the acute proinflammatory phase and the immunosuppression phase to reduce sepsis progression. This was accomplished by precisely incorporating SAHA into polymeric nanoparticles for delivery into immune cells and into sepsis-induced mice models. HDACi administration—including SAHA—can reduce inflammation and prevent cell death and apoptosis by increasing both nuclear and cytoplasmic acetylation. The results showed that iNP-SAHA was highly effective at reducing proinflammatory cytokine serum levels and demonstrated significantly higher long-term survival in an LPS-induced endotoxemia mouse model. Future development of nanoparticle delivered HDAC inhibitors has the potential to address the biphasic nature of sepsis. The versatility in this platform can allow for translation to other inflammatory disease models by utilizing other therapeutic cargos and thus improving their pharmacological therapy.

The use of nano-drug delivery systems to incorporate SAHA aids to overcome inherent drug limitations, such as poor water solubility, short half-life, and toxicity. When internalized by cells, the use of 50:50 PLGA allowed for faster release of SAHA from the pro-drug conjugates due to the increase in glycolic acid proportions as compared to using PLA. Insights into cellular uptake of iNPs have been well explored, such as studies from Sankar et. al which revealed the internalization of SAHA loaded PLGA nanoparticles by A549 lung cancer cells. With this, no further investigation into iNP uptake was needed. To further improve delivery to phagocytic cells, nanoconjugate and nanoparticle emulsification methods was utilized to generate iNP-SAHA as these conjugates imparted a delayed mechanism of release for SAHA compared to conventional encapsulation methods. The use of PLA allowed for delayed pro-drug conjugate release within the cell, and the use of PLGA allowed for rapid release of SAHA.

Furthermore, it has been shown that loading SAHA into polymeric-based nanoparticles are biocompatible within mice models, along with improving drug delivery and distribution into various organ systems. The results showed that iNP-SAHA was highly effective at reducing proinflammatory cytokine serum levels and demonstrated significantly higher long-term survival in an LPS-induced endotoxemia mouse model. An inhibition of plasma TNFα was observed following intraperitoneal iNP-SAHA administration. However, IL-6 was not significantly reduced in vivo contrary to the in vitro assays. Previous studies using human monocytes have found that IL-6 secretions were inhibited under high lactic acid (LA) concentrations. LA can exert differential and opposing immunomodulatory functions in a cell type-dependent manner. Further, it was shown that TNFα was more affected than IL-6, suggesting that LA may reduce TNFα but not IL-6 secretion in vivo under pathophysiological conditions. To corroborate the current study, the previous study showed that intravenous administration of iNP followed by isolation of splenocytes and subsequent stimulation with LPS or CpG ODN resulted in significant decreases in IL-6, MCP-1, and TNFα. Moreover, histological analysis showed no remarkable changes in spleen or liver organ structure in iNP or iNP-SAHA treated mice. These findings demonstrate not only the safety and tolerability of iNP-SAHA in animal models, but their effectiveness at attenuating acute hyperinflammation and protecting against LPS-induced septic shock.

The pleiotropic effects of SAHA, a pan-HDACi, is due to its non-specific inhibition of HDAC enzymes, which could result in enhanced toxicity and undesirable side effects, such as thrombocytopenia and neutropenia, that limits its effective long term clinical use. This, and some cases of cardiotoxicity have tempered enthusiasm towards pan-HDACis, which has motivated researchers to discover isoform-selective inhibitors that are anticipated to have fewer adverse effects. With this, perhaps incorporation of a more selective HDACi isoform into nanoparticles could promote precise epigenetic tuning to reduce off-target effects. Future investigation into using HDAC6 selective inhibitors may prove to be more efficacious at addressing the dysregulated responses seen in sepsis.

### Example 3: Chemically Modified Suberoylanilide Hydroxamic Acid and Nanoparticle-Based Immunotherapy to Mitigate Immune Dysregulation in Sepsis

### Methods

Differences from the methods sections of Example 1 and Example 2 are outlined below. The remaining methods used in Example 3 are identical to the above examples.

Preparation of iNP and iNP-SAHA and Characterization Via DLS

iNP and iNP-SAHA were prepared by the oil-in-water (o/w) emulsion solvent evaporation (SE) technique using a similar method as described. Briefly, 200 mg of acid-terminated PLA was dissolved in ethyl acetate to a concentration of 80 mg/mL to generate iNP. For iNP-SAHA, SAHA loading was determined from the coupling efficiency via NMR. Pre-determined amounts of PLGA-SAHA were added to PLA at 50 mg/mL in ethyl acetate to formulate three loadings of SAHA into iNPs: 3.27 μg/mg iNP-SAHA_{Low}, 9.81 μg/mg iNP-SAHA_{Med}, and 29.43 μg/mg iNP-SAHA_{High}. iNP and iNP-SAHA synthesis was similarly performed, where 20 mL of 1% PEMA was added and sonicated at 100% amplitude for 30 seconds using a Cole-Parmer 500-Watt Ultrasonic Homogenizer. The resulting o/w emulsion was then added to 80 mL of magnetically stirred 0.5% PEMA overnight until all the ethyl acetate evaporated. The nanoparticles were then collected by centrifugation at 12,000×g for 20 min at 4° C. and washed with 40 mL of 0.1M sodium bicarbonate/carbonate buffer. The centrifugation and washing steps were repeated two more times. The final wash cycle used MilliQ. Water to remove excess sodium bicarbonate buffer. A mixture of sucrose and mannitol was added to the particle suspension as cryoprotectants to achieve a final concentration of 4% and 3% w/v, respectively. The nanoparticles were then frozen at −80° C. and lyophilized for at least 2 days prior to use. The size and zeta potential of the nanoparticles were determined by dynamic light scattering (DLS) by mixing 10 μL of a 10 mg/mL particle solution into 990 μL of MilliQ water using a Malvern Zetasizer Nano ZSP (Malvern Instruments Inc., Westborough MA). For these measurements, iNPs were dispersed in water at pH 6 at room temperature. The Z-average sizes were recorded as the average of at least three measurements.

iNP-SAHA Loading and Controlled Release Measured Through UPLC

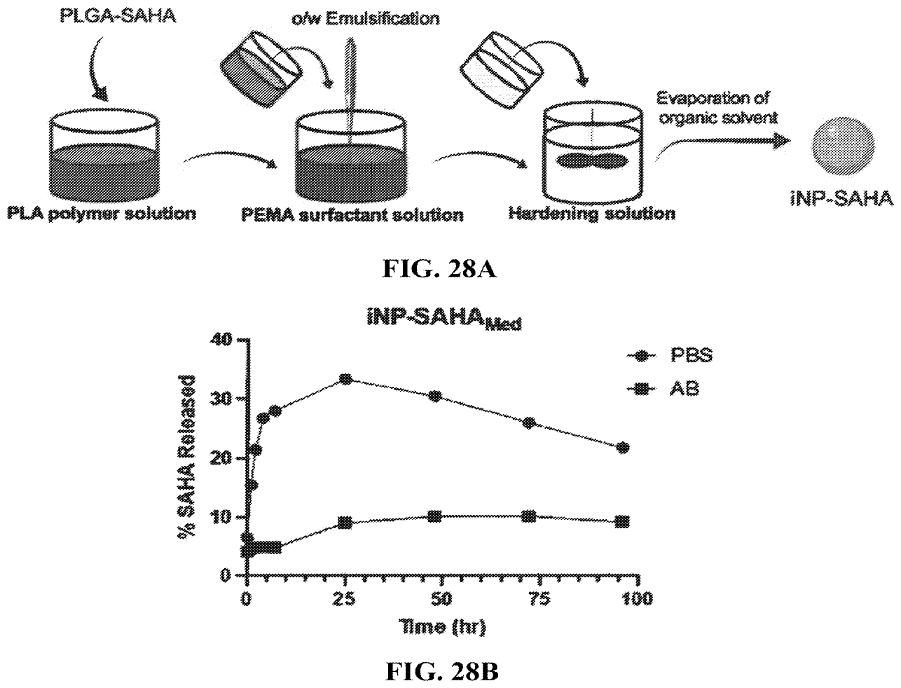

The release of SAHA from iNP-SAHA was measured over 96 hours. iNP-SAHA was dispersed at 5 mg/mL in either acetate buffer (pH 5) or PBS (pH 7.4) supplemented with 10% methanol to assess the impact of pH on SAHA-OH liberation from the PLGA-SAHA conjugates and incubated at 37° C. At pre-determined time points, iNP-SAHA was centrifuged at 12,000×g for 5 min and the 300 μL of the supernatant was collected and 300 μL of fresh buffer was added following resuspension of the pellet and incubated at 37° C. until the following timepoint. All supernatant samples were measured via UPLC after collection. After the final time point, the pellet of iNP-SAHA was dissolved in 1N NaOH and the total amount of remaining SAHA was determined. SAHA-OH concentration was determined using the Acquity UPLC and detected at 254 nm. Gradient mobile phase of (A) 0.1% TFA in H2O and (B) 0.1% TFA in methanol were followed as such: 0 min, 75% A 25% B; 9 min, 35% A 65% B; 10 min 35% A 65% B; 10.1 min 75% A 25% B. Flow rate of 1 mL/min processed through a C18 column (5 μm, 4.6×150 mm, Thermo Scientific). The release of SAHA from iNP-SAHA_{Med} is shown in FIG. 28B.

### Results

Synthesis and Preparation of Immunomodulatory SAHA Nanoparticles (iNP-SAHA)

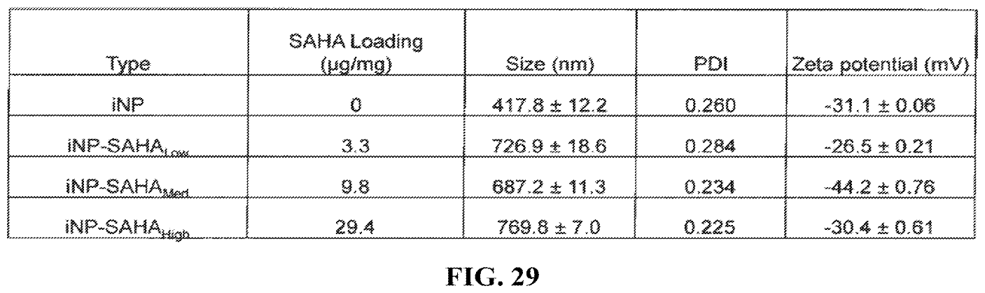

SAHA-OH was conjugated to carboxyl-terminated PLGA (PLGA-SAHA) via an EDC/NHS reaction and confirmed to have a 77.5% coupling efficiency by ¹H-NMR. Three different loadings of iNP-SAHAs were prepared by mixing the PLGA-SAHA conjugate with unmodified PLA polymer at precise stoichiometric ratios via single oil-in-water (o/w) emulsion technique (FIG. 28A). iNP-SAHA loading and physiochemical characterization, as measured through the UPLC and DLS, determined the actual SAHA loadings (μg/mg), particle size characteristics between 400-800 nm, PDI less than 0.3, and zeta potentials between −26-44 mV (FIG. 29). These subsequent iNP-SAHAs were further optimized and evaluated in BMMØs.

iNP-SAHA Mitigates Proinflammatory Cytokine Responses Upon LPS Challenge and Reduces Macrophage Apoptosis

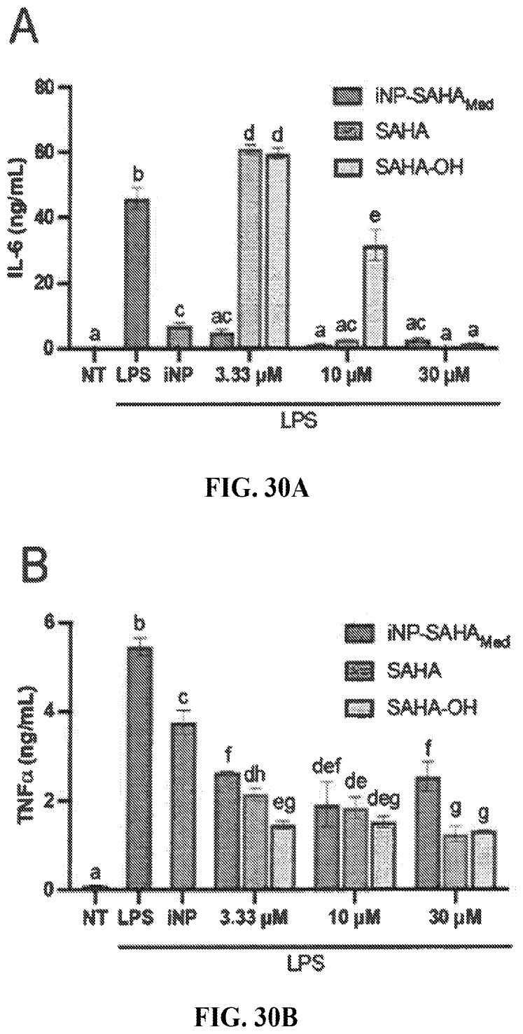

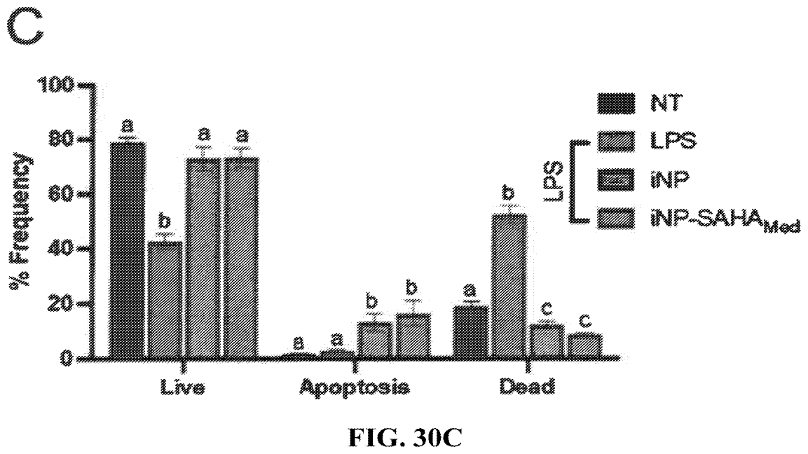

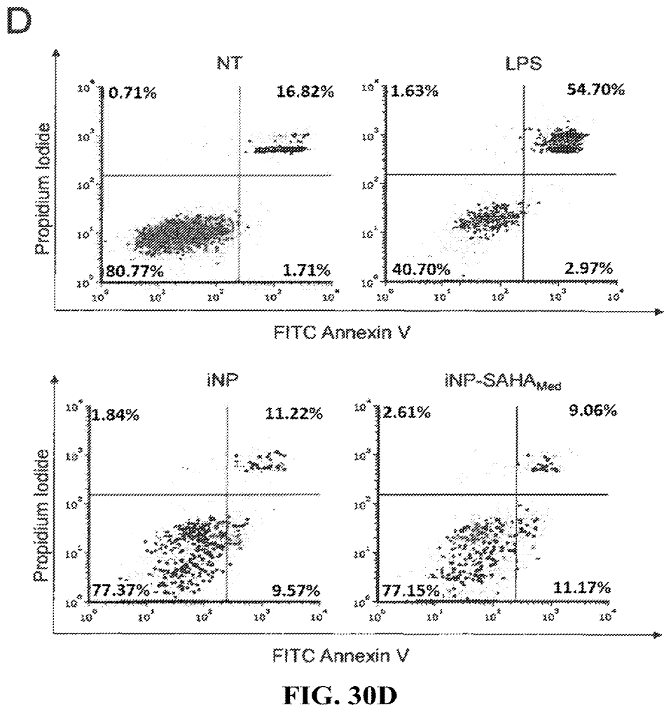

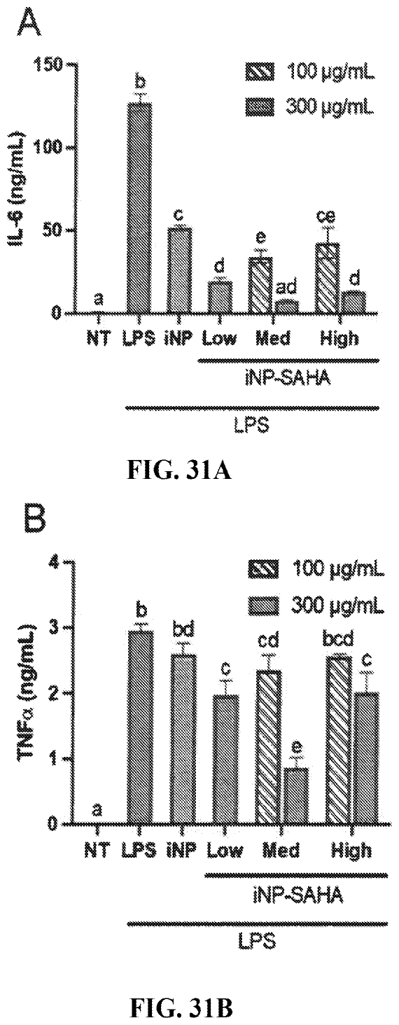

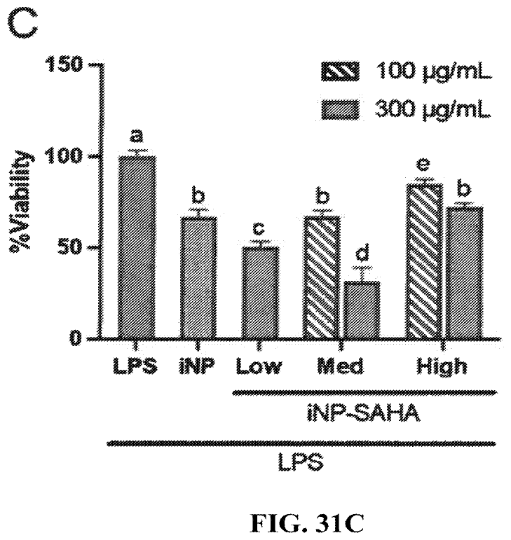

All iNP-SAHA formulations performed significantly better than cargo-less iNPs at suppressing proinflammatory cytokine secretions from BMMØs stimulated with LPS (FIGS. 30A-30B). All iNP-SAHA and iNP formulations were treated at 300 μg/mL, where this concentration was deemed the most effective (FIGS. 31A-31C). Interestingly, it was observed that the most effective iNP-SAHA (NP-SAHA_{Me}d) did not contain the highest concentration of SAHA, whereas iNP-SAHA_{Low} was less effective (FIGS. 31A-31C). iNP-SAHA_{Med} significantly reduced IL-6 secretions as compared to soluble SAHA or SAHA-OH controls, yet this effect was not as pronounced for TNFα. Further, iNP-SAHA_{Med} significantly improved viability and almost completely reversed apoptosis and necrosis in BMMØs treated with LPS (FIGS. 30C-30D). Despite iNP-SAHA's ability to better suppress proinflammatory cytokines, there were no statistical differences in its ability to prevent apoptosis or necrosis compared to iNPs (FIGS. 30C-30D). These results demonstrated that iNP-SAHA could simultaneously reduce proinflammatory and apoptotic responses induced by LPS, offering a synergistic delivery system to restore the multiple dysregulated immune responses due to sepsis.

### Discussion

Epigenetic mechanisms such as DNA methylation, histone modifications, and non-coding RNAs are perturbed in sepsis and are associated with increased mortality due to their contributions to long-term immunosuppression. An autopsy study identified persistent foci of infection and microabcesses in 80% of septic individuals demonstrating immunocompromised status at death. Further, dramatic losses of leukocytes by apoptosis results in the propagation of septic complications ultimately leading to reduced patient survival. Moreover, SAHA induces apoptosis by triggering both the extrinsic and intrinsic apoptotic pathways through upregulation and/or downregulation of various apoptotic genes. The ability of SAHA to induce apoptosis in primary macrophages was recapitulated; yet interestingly, the modified SAHA “SAHA-OH” did not elicit similar effects (FIG. 11D). This modification to SAHA not only facilitated its covalent conjugation to PLGA for incorporation into iNPs, but greatly improved its toxicity profile. This serendipitous finding where SAHA-OH lacks the ability to induce apoptosis will aid in reducing apoptosis-induced immunosuppression that is seen during sepsis progression. Furthermore, the results also demonstrated that the incorporation of SAHA-OH into iNPs “iNP-SAHA” possessed a favorable reduction in apoptosis, thus furthering its ability to combat the loss of leukocytes through apoptosis (FIG. 30C).

As shown in Table 1, iNP-SAHA were optimized and formulated with precise physicochemical properties ideal for delivery to phagocytic immune cells. Insights into cellular uptake of iNPs have been well explored, such as studies from Sankar et. al which exhibited the internalization of SAHA loaded PLGA nanoparticles by A549 lung cancer cells. With this, no further investigation into iNP uptake was needed. Analysis into the in vitro biological function of iNP-SAHA demonstrated anti-inflammatory properties through the suppression of pro-inflammatory cytokine mediators along with improving cellular viability upon LPS challenge (FIGS. 30A-30D). Furthermore, it has been shown that loading SAHA into polymeric-based nanoparticles are biocompatible within mice models, along with improving drug delivery and distribution into various organ systems.

While the precise mechanism by which SAHA reverses genotypic and phenotypic characteristics of sepsis is unknown, it is believed that SAHA may allow for the regulation in acetylation profiles within cells. During severe septic shock, a global cellular hypoacetylation effect occurs where SAHA can be utilized to restore acetylation homeostasis. SAHA has also shown to prevent epigenetic changes in gene expression, thus maintaining the “status quo.” Confoundingly, SAHA down-regulated genes involved in co-stimulation and cytokine production of BMMØs in response to TLR agonists. Specifically, studies from Li et. al, Zhao et. al, and Chong et. al have demonstrated that SAHA has direct effects on the various components of the LPS/TLR4-MyD88 dependent pathway through the inhibition of MyD88 expression via the acetylation of STAT-1, IRAK1, and NF-xB proteins.

The pleiotropic effects of SAHA, a pan-HDACi, is due to its non-specific inhibition of HDAC enzymes, which could result in enhanced toxicity and undesirable side effects that limits its effective clinical use. This, and some cases of cardiotoxicity have tempered enthusiasm towards pan-HDACis, which has motivated researchers to discover isoform-selective inhibitors that are anticipated to have fewer adverse effects. Further, there is no systematic study on the effects of pan-HDAC inhibition through increasing HDAC selective inhibition in models of sepsis. With this, perhaps incorporation of a more selective HDACi isoform into nanoparticles could promote precise epigenetic tuning to reduce off-target effects. Future investigation into using HDAC6 selective inhibitors may prove to be more efficacious at addressing the dysregulated responses seen in sepsis.

Here, the HDACi nanoparticle platform has the capabilities to target the acute proinflammatory phase and the immunosuppression phase to reduce sepsis progression. This was accomplished by precisely incorporating SAHA into polymeric nanoparticles for delivery into immune cells and into sepsis-induced mice models. HDACi administration—including SAHA—can reduce inflammation, prevent cell death and apoptosis, and improve survival by increasing both nuclear and cytoplasmic acetylation. Future development of nanoparticle delivered HDAC inhibitors has the potential to address the biphasic nature of sepsis. The versatility in this platform can allow for translation to other inflammatory disease models by utilizing other therapeutic cargos and thus improving their pharmacological therapy.

### REFERENCES

(1) von Knethen, A.; Brune, B. Histone Deacetylation Inhibitors as Therapy Concept in Sepsis. International Journal of Molecular Sciences. MDPI AG Jan. 2, 2019.
(2) Li, Y; Liu, B.; Fukudome, E. Y; Kochanek, A. R.; Finkelstein, R. A.; Chong, W.; Jin, G.; Lu, J.; Demoya, M. A.; Velmahos, G. C.; Alam, H. B. Surviving Lethal Septic Shock without Fluid Resuscitation in a Rodent Model. Surgery 2010, 148 (2), 246-254.
(3) AC, W.; RW, J. New and Emerging HDAC Inhibitors for Cancer Treatment. J Clin Invest 2014, 124 (1), 30-39.
(4) Kim, H. J.; Bae, S. C. Histone Deacetylase Inhibitors: Molecular Mechanisms of Action and Clinical Trials as Anti-Cancer Drugs. American Journal of Translational Research. e-Century Publishing Corporation 2011, pp 166-179.
(5) Nebbioso, A.; Clarke, N.; Voltz, E.; Germain, E.; Ambrosino, C.; Bontempo, P.; Alvarez, R.; Schiavone, E. M.; Ferrara, F.; Bresciani, F.; Weisz, A.; de Lera, A. R.; Gronemeyer, H.; Altucci, L. Tumor-Selective Action of HDAC Inhibitors Involves TRAIL Induction in Acute Myeloid Leukemia Cells. NATURE MEDICINE 2005, 11 (1), 77.
(6) Shao, Y; Gao, Z.; Marks, P. A.; Jiang, X. Apoptotic and Autophagic Cell Death Induced by Histone Deacetylase Inhibitors. Proc Natl Acad Sci USA 2004, 101 (52), 18030-18035.
(7) Shah, R. R. Safety and Tolerability of Histone Deacetylase (HDAC)
(8) Kankaanranta, H.; Janka-Junttila, M.; Ilmarinen-Salo, P.; Ito, K.; Jalonen, U.; Ito, M.; Adcock, I. M.; Moilanen, E.; Zhang, X. Histone Deacetylase Inhibitors Induce Apoptosis in Human Eosinophils and Neutrophils. Journal of Inflammation 2010, 7 (1), 1-15.
(9) West, A. C.; Smyth, M. J.; Johnstone, R. W. The Anticancer Effects of HDAC Inhibitors Require the Immune System. OncoImmunology 2014, 3 (1).
(10) Gupta, P.; Reid, R. C.; Iyer, A.; Sweet, M. J.; Fairlie, D. P. Towards Isozyme-Selective HDAC Inhibitors For Interrogating Disease; 2012; Vol. 12.
(11) Subramanian, S.; Bates, S. E.; Wright, J. J.; Espinoza-Delgado, I.; Piekarz, R. L. Clinical Toxicities of Histone Deacetylase Inhibitors. Pharmaceuticals 2010, 3 (9), 2751-2767.
(12) Leoni, F.; Zaliani, A.; Bertolini, G.; Porro, G.; Pagani, P.; Pozzi, P.; Donà, G.; Fossati, G.; Sozzani, S.; Azam, T.; Bufler, P.; Fantuzzi, G.; Goncharov, I.; Kim, S. H.; Pomerantz, B. J.; Reznikov, L. L.; Siegmund, B.; Dinarello, C. A.; Mascagni, P. The Antitumor Histone Deacetylase Inhibitor Suberoylanilide Hydroxamic Acid Exhibits Antiinflammatory Properties via Suppression of Cytokines. Proc Natl Acad Sci USA 2002, 99 (5), 2995-3000.
(13) Li, Y; Liu, B.; Zhao, H.; Sailhamer, E. A.; Fukudome, E. Y; Zhang, X.; Kheirbek, T.; Finkelstein, R. A.; Velmahos, G. C.; Demoya, M.; Hales, C. A.; Alam, H. B. Protective Effect of Suberoylanilide Hydroxamic Acid against Lps-Induced Septic Shock in Rodents. Shock 2009, 32 (5), 517-523.
(14) Zhao, Y; Zhou, P.; Liu, B.; Bambakidis, T.; Mazitschek, R.; Alam, H. B.; Li, Y Protective Effect of Suberoylanilide Hydroxamic Acid against Lipopolysaccharide-Induced Liver Damage in Rodents. Journal of Surgical Research 2015, 194 (2), 544-550.
(15) Chong, W.; Li, Y; Liu, B.; Zhao, T.; Fukudome, E. Y; Liu, Z.; Smith, W. M.; Velmahos, G. C.; Demoya, M. A.; Alam, H. B. Histone Deacetylase Inhibitor Suberoylanilide Hydroxamic Acid Attenuates Toll-like Receptor 4 Signaling in Lipopolysaccharide-Stimulated Mouse Macrophages. Journal of Surgical Research 2012, 178 (2), 851-859.
(16) Li, Y; Zhao, T.; Liu, B.; Halaweish, I.; Mazitschek, R.; Duan, X.; Alam, H. B. Inhibition of Histone Deacetylase 6 Improves Long-Term Survival in a Lethal Septic Model. J Trauma Acute Care Surg 2015, 78 (2), 378.
(17) Negmeldin, A. T.; Pflum, M. K. H. The Structural Requirements of Histone Deacetylase Inhibitors: SAHA Analogs Modified at the C5 Position Display Dual HDAC6/8 Selectivity. Bioorganic & Medicinal Chemistry Letters 2017, 27 (15), 3254-3258.
(18) Negmeldin, A. T.; Padige, G.; Bieliauskas, A. v.; Pflum, M. K. H. Structural Requirements of HDAC Inhibitors: SAHA Analogues Modified at the C2 Position Display HDAC6/8 Selectivity. ACS Medicinal Chemistry Letters 2017, 8 (3), 281-286.
(19) Chan, A. M.; Fletcher, S. Shifting the Paradigm in Treating Multi-Factorial Diseases: Polypharmacological Co-Inhibitors of HDAC6. RSC Medicinal Chemistry. Royal Society of Chemistry 2021, pp 178-196.
(20) Melesina, J.; Praetorius, L.; Simoben, C. v.; Robaa, D.; Sippl, W. Design of Selective Histone Deacetylase Inhibitors: Rethinking Classical Pharmacophore. Future Medicinal Chemistry. Future Medicine Ltd. 2018, pp 1537-1540.
(21) Senger, J.; Melesina, J.; Marek, M.; Romier, C.; Oehme, I.; Witt, O.; Sippl, W.; Jung, M. Synthesis and Biological Investigation of Oxazole Hydroxamates as Highly Selective Histone Deacetylase 6 (HDAC6) Inhibitors. Journal of Medicinal Chemistry 2016, 59 (4), 1545-1555.
(22) Heimburg, T.; Chakrabarti, A.; Lancelot, J.; Marek, M.; Melesina, J.; Hauser, A. T.; Shaik, T. B.; Duclaud, S.; Robaa, D.; Erdmann, F.; Schmidt, M.; Romier, C.; Pierce, R. J.; Jung, M.; Sippl, W. Structure-Based Design and Synthesis of Novel Inhibitors Targeting HDAC8 from Schistosoma mansoni for the Treatment of Schistosomiasis. Journal of Medicinal Chemistry 2016, 59 (6), 2423-2435.
(23) Butler, K.; Kalin, J.; Brochier, C.; Vistoli, G.; Langley, B.; Kozikowski, A. Rational Design and Simple Chemistry Yield a Superior, Neuroprotective HDAC6 Inhibitor, Tubastatin A. J Am Chem Soc 2010, 132 (31), 10842-10846.
(24) Goehringer, N.; Peng, Y; Nitzsche, B.; Biermann, H.; Pradhan, R.; Schobert, R.; Herling, M.; Höpfner, M.; Biersack, B. Improved Anticancer Activities of a New Pentafluorothio-Substituted Vorinostat-Type Histone Deacetylase Inhibitor. Pharmaceuticals 2021, 14 (12). Porter, N. J.; Mahendran, A.; Breslow, R.; Christianson, D. W. Unusual Zinc-Binding Mode of HDAC6-Selective Hydroxamate Inhibitors. Proc Natl Acad Sci USA 2017, 114 (51), 13459-13464.
(25) Wu, H.; Yang, K.; Zhang, Z.; Leisten, E. D.; Li, Z.; Xie, H.; Liu, J.; Smith, K. A.; Novakova, Z.; Barinka, C.; Tang, W. Development of Multifunctional Histone Deacetylase 6 Degraders with Potent Antimyeloma Activity. Journal of Medicinal Chemistry 2019, 62 (15), 7042-7057.
(26) Bass, A. K. A.; El-Zoghbi, M. S.; Nageeb, E. M.; Mohamed, M. F. A.; Badr, M.; Abuo-Rahma, G. E. A. Comprehensive Review for Anticancer Hybridized Multitargeting HDAC Inhibitors. Eur J Med Chem 2021, 209.
(27) Casey, L. M.; Kakade, S.; Decker, J. T.; Rose, J. A.; Deans, K.; Shea, L. D.; Pearson, R. M. Cargo-Less Nanoparticles Program Innate Immune Cell Responses to Toll-like Receptor Activation. Biomaterials 2019, 218.
(28) Lasola, J. J. M.; Cottingham, A. L.; Scotland, B. L.; Truong, N.; Hong, C. C.; Shapiro, P.; Pearson, R. M. Immunomodulatory Nanoparticles Mitigate Macrophage Inflammation via Inhibition of PAMP Interactions and Lactate-Mediated Functional Reprogramming of NF-KB and P38 MAPK. Pharmaceutics 2021, 13 (11), 1841.
(29) Deng, Q.; Zhao, T.; Pan, B.; Dennahy, I. S.; Duan, X.; Williams, A. M.; Liu, B.; Lin, N.; Bhatti, U. F.; Chen, E.; Alam, H. B.; Li, Y Protective Effect of Tubastatin A in CLP-Induced Lethal Sepsis. Inflammation 2018, 41 (6), 2101-2109.
(30) Stammler, D.; Eigenbrod, T.; Menz, S.; Frick, J. S.; Sweet, M. J.; Shakespear, M. R.; Jantsch, J.; Siegert, I.; Wolfle, S.; Langer, J. D.; Oehme, I.; Schaefer, L.; Fischer, A.; Knievel, J.; Heeg, K.; Dalpke, A. H.; Bode, K. A. Inhibition of Histone Deacetylases Permits Lipopolysaccharide-Mediated Secretion of Bioactive IL-1 via a Caspase-1-Independent Mechanism. The Journal of Immunology 2015, 195 (11), 5421-5431.
(31) Liliang, J.; Batra, S.; Douda, D. N.; Palaniyar, N.; Jeyaseelan, S. CXCL1 Contributes to Host Defense in Polymicrobial Sepsis via Modulating T Cell and Neutrophil Functions. J Immunol 2014, 193 (7), 3549.
(32) Eckschlager, T.; Plch, J.; Stiborova, M.; Hrabeta, J. Histone Deacetylase Inhibitors as Anticancer Drugs. International Journal of Molecular Sciences. MDPI AG Jul. 1, 2017.
(33) Woan, K. v.; Sahakian, E.; Sotomayor, E. M.; Seto, E.; Villagra, A. Modulation of Antigen-Presenting Cells by HDAC Inhibitors: Implications in Autoimmunity and Cancer. Immunology and Cell Biology. January 2012, pp 55-65.
(34) Hermida, M.; de Melo, C.; Lima, I.; de Sa Oliveira, G.; dos-Santos, W. Histological Disorganization of Spleen Compartments and Severe Visceral Leishmaniasis. Frontiers in Cellular and Infection Microbiology 2018, 8 (394).
(35) Li, Y; Seto, E. HDACs and HDAC Inhibitors in Cancer Development and Therapy. Cold Spring Harbor Perspectives in Medicine 2016, 6 (10).
(36) Bode, K.; Schroder, K.; Hume, D. A.; Ravasi, T.; Heeg, K.; Sweet, M. J.; Dalpke, A. H. Histone Deacetylase Inhibitors Decrease Toll-like Receptor-Mediated Activation of Proinflammatory Gene Expression by Impairing Transcription Factor Recruitment. Immunology 2007, 122 (4), 596-606.
(37) Kroesen, M.; Gielen, P.; Brok, I. C.; Armandari, I.; Hoogerbrugge, P. C.; Adema, G. J. HDAC Inhibitors and Immunotherapy; a Double Edged Sword?Oncotarget 2014, 5 (16), 6558-6572.
(38) Fairlie, D. P.; Sweet, M. J. HDACs and Their Inhibitors in Immunology: Teaching Anticancer Drugs New Tricks. Immunology and Cell Biology 2012, 90 (1), 3-5.
(39) Grabiec, A. M.; Tak, P. P.; Reedquist, K. A. Targeting Histone Deacetylase Activity in Rheumatoid Arthritis and Asthma as Prototypes of Inflammatory Disease: Should We Keep Our HATs On? Arthritis Research & Therapy 2008, 10 (5), 226.
(40) Roger, T.; Me Lugrin, J.; Roy, D. le; Ve Goy, G.; Mombelli, M.; Koessler, T.; Ding, X. C.; Chanson, A.-L.; Reymond, M. K.; Miconnet, I.; Schrenzel, J.; François, P.; Calandra, T. Histone Deacetylase Inhibitors Impair Innate Immune Responses to Toll-like Receptor Agonists and to Infection. Blood 2011, 117 (4), 1205-1217.
(41) Vishwakarma, S.; Iyer, L. R.; Muley, M.; Singh, P. K.; Shastry, A.; Saxena, A.; Kulathingal, J.; Vijaykanth, G.; Raghul, J.; Rajesh, N.; Rathinasamy, S.; Kachhadia, V.; Kilambi, N.; Rajgopal, S.; Balasubramanian, G.; Narayanan, S. Tubastatin, a Selective Histone Deacetylase 6 Inhibitor Shows Anti-Inflammatory and Anti-Rheumatic Effects. International Immunopharmacology 2013, 16 (1), 72-78.
(42) Ripamonti, C.; Spadotto, V; Pozzi, P.; Stevenazzi, A.; Vergani, B.; Marchini, M.; Sandrone, G.; Bonetti, E.; Mazzarella, L.; Minucci, S.; Steinkuhler, C.; Fossati, G. HDAC Inhibition as Potential Therapeutic Strategy to Restore the Deregulated Immune Response in Severe COVID-19. Frontiers in Immunology 2022, 13.
(43) Li, Y; Liu, B.; Gu, X.; Kochanek, A. R.; Fukudome, E. Y; Liu, Z.; Zhao, T.; Chong, W.; Zhao, Y; Zhang, D.; Libermann, T. A.; Alam, H. B. Creating a “pro-Survival” Phenotype through Epigenetic Modulation. Surgery (United States) 2012, 152 (3), 455-464.
(44) Hull, E. E.; Montgomery, M. R.; Leyva, K. J. HDAC Inhibitors as Epigenetic Regulators of the Immune System: Impacts on Cancer Therapy and Inflammatory Diseases. 2016.
(45) Gatla, H. R.; Muniraj, N.; Thevkar, P.; Yavvari, S.; Sukhavasi, S.; Makena, M. R. Regulation of Chemokines and Cytokines by Histone Deacetylases and an Update on Histone Decetylase Inhibitors in Human Diseases. International Journal of Molecular Sciences 2019, 20 (5).
(46) Füllgrabe, J.; Hajji, N.; Joseph, B. Cracking the Death Code: Apoptosis-Related Histone Modifications. Cell Death and Differentiation. August 2010, pp 1238-1243.
(47) Delano, M.; Ward, P. The Immune System's Role in Sepsis Progression, Resolution, and Long-Term Outcome. Immunological Reviews 2016, 330-352.
(48) Hotchkiss, R. S.; Swanson, P. E.; Michael Knudson, C.; Chang, K. C.; Perren Cobb, J.; Osborne, D. F.; Zollner, K. M.; Buchman, T. G.; Korsmeyer, S. J.; Karl, I. E. Overexpression of Bcl-2 in Transgenic Mice Decreases Apoptosis and Improves Survival in Sepsis. J Immunol 1999.
(49) Wesche-Soldato, D. E.; Swan, R. Z.; Chung, C.-S.; Ayala, A.; Surg, D.; Res, /. The Apoptotic Pathway as a Therapeutic Target in Sepsis.
(50) Waibel, M.; Christiansen, A. J.; Hibbs, M. L.; Shortt, J.; Jones, S. A.; Simpson, I.; Light, A.; O'Donnell, K.; Morand, E. F.; Tarlinton, D. M.; Johnstone, R. W.; Hawkins, E. D. Manipulation of B-Cell Responses with Histone Deacetylase Inhibitors. Nature Communications 2015, 6.
(51) Chaudhry, H.; Zhou, J.; Zhong, Y; Ali, M. M.; Mcguire, F.; Nagarkatti, P. S.; Nagarkatti, M. Role of Cytokines as a Double-Edged Sword in Sepsis.
(52) Zhang, L.; Jin, S.; Wang, C.; Jiang, R.; Wan, J. Histone Deacetylase Inhibitors Attenuate Acute Lung Injury During Cecal Ligation and Puncture-Induced Polymicrobial Sepsis. World J Surg 2010, 34, 1676-1683.
(53) Weischenfeldt, J.; Porse, B. Bone Marrow-Derived Macrophages (BMM): Isolation and Applications. Cold Spring Harbor Protocols 2008, 3 (12), pdb.prot5080.
(54) Feldman, A. T.; Wolfe, D. Tissue Processing and Hematoxylin and Eosin Staining. Methods in Molecular Biology 2014, 1180, 31-43.
(55) Rieppo, L.; Janssen, L.; Rahunen, K.; Lehenkari, P.; Finnilä, M. A. J.; Saarakkala, S. Histochemical Quantification of Collagen Content in Articular Cartilage. PLOS One 2019, 14 (11).
(56) Nebbioso, A.; Clarke, N.; Voltz, E.; Germain, E.; Ambrosino, C.; Bontempo, P.; Alvarez, R.; Schiavone, E. M.; Ferrara, F.; Bresciani, F.; Weisz, A.; de Lera, A. R.; Gronemeyer, H.; Altucci, L., “Tumor-Selective Action of HDAC Inhibitors Involves TRAIL Induction in Acute Myeloid Leukemia Cells,” NATURE MEDICINE 2005, 11 (1), 77
(57) Shao, Y.; Gao, Z.; Marks, P. A.; Jiang, X. Apoptotic and Autophagic Cell Death Induced by Histone Deacetylase Inhibitors. Proceedings of the National Academy of Sciences of the United Stated of America 2004, 101 (52), 18030-18035.
(58) Zhao, Y.; Zhou, P.; Liu, B.; Bambakidis, T.; Mazitschek, R.; Alam, H. B.; Li, Y. Protective Effect of Suberoylanilide Hydroxamic Acid against Lipopolysaccharide-Induced Liver Damage in Rodents. Journal of Surgical Research 2015, 194 (2), 544-550.
(59) Li, Y.; Liu, B.; Fukudome, E. Y.; Kochanek, A. R.; Finkelstein, R. A.; Chong, W.; Jin, G.; Lu, J.; Demoya, M. A.; Velmahos, G. C.; Alam, H. B. Surviving Lethal Septic Shock without Fluid Resuscitation in a Rodent Model. Surgery 2010, 148 (2), 246-254.
(60) Chong, W.; Li, Y.; Liu, B.; Zhao, T.; Fukudome, E. Y.; Liu, Z.; Smith, W. M.; Velmahos, G. C.; Demoya, M. A.; Alam, H. B. Histone Deacetylase Inhibitor Suberoylanilide Hydroxamic Acid Attenuates Toll-like Receptor 4 Signaling in Lipopolysaccharide-Stimulated Mouse Macrophages. Journal of Surgical Research 2012, 178 (2), 851-859.
(61) Gupta, P.; Reid, R. C.; Iyer, A.; Sweet, M. J.; Fairlie, D. P. Towards Isozyme-Selective HDAC Inhibitors For Interrogating Disease; 2012; Vol. 12.
(62) Casey, L. M.; Kakade, S.; Decker, J. T.; Rose, J. A.; Deans, K.; Shea, L. D.; Pearson, R. M. Cargo-Less Nanoparticles Program Innate Immune Cell Responses to Toll-like Receptor Activation. Biomaterials 2019, 218.
(63) Subramanian, S.; Bates, S. E.; Wright, J. J.; Espinoza-Delgado, I.; Piekarz, R. L. Clinical Toxicities of Histone Deacetylase Inhibitors. Pharmaceuticals. MDPI AG 2010, pp 2751-2767.
(64) Roger, T.; Me Lugrin, J.; Roy, D. le; Ve Goy, G.; Mombelli, M.; Koessler, T.; Ding, X. C.; Chanson, A.-L.; Reymond, M. K.; Miconnet, I.; Schrenzel, J.; Frangois, P.; Calandra, T. Histone Deacetylase Inhibitors Impair Innate Immune Responses to Toll-like Receptor Agonists and to Infection. Blood 2011, 117 (4), 1205-1217.
(65) Carson, W. F.; Cavassani, K. A.; Dou, Y.; Kunkel, S. L. Epigenetic Regulation of Immune Cell Functions during Post-Septic Immunosuppression. Epigenetics 2011, 6 (3), 273-283. Torgersen, C.; Moser, P.; Luckner, G.; Mayr, V.; Jochberger, S.; Hasibeder, W. R.; Dünser, M. W. Macroscopic Postmortem Findings in 235 Surgical Intensive Care Patients with Sepsis. Anesthesia and Analgesia 2009, 108 (6), 1841-1847.
(66) Zhao, J.; Stenzel, M. H. Entry of Nanoparticles into Cells: The Importance of Nanoparticle Properties. Polymer Chemistry. Royal Society of Chemistry Jan. 21, 2018, pp 259-272. Sankar, R.; Ravikumar, V. Biocompatibility and Biodistribution of Suberoylanilide Hydroxamic Acid Loaded Poly (DL-Lactide-Co-Glycolide) Nanoparticles for Targeted Drug Delivery in Cancer. Biomedicine & pharmacotherapy 2014, 68 (7), 865-871.
(67) Li, Y.; Liu, B.; Gu, X.; Kochanek, A. R.; Fukudome, E. Y.; Liu, Z.; Zhao, T.; Chong, W.; Zhao, Y.; Zhang, D.; Libermann, T. A.; Alam, H. B. Creating a “pro-Survival” Phenotype through Epigenetic Modulation. Surgery (United States) 2012, 152 (3), 455-464.
(68) Binnie, A.; Tsang, J. L. Y.; Hu, P.; Carrasqueiro, G.; Castelo-Branco, P.; dos Santos, C. C. Epigenetics of Sepsis. Critical Care Medicine. Lippincott Williams and Wilkins 2020, pp 745-756.
(69) Bode, K. A.; Schroder, K.; Hume, D. A.; Ravasi, T.; Heeg, K.; Sweet, M. J.; Dalpke, A. H. Histone Deacetylase Inhibitors Decrease Toll-like Receptor-Mediated Activation of Proinflammatory Gene Expression by Impairing Transcription Factor Recruitment. Immunology 2007, 122 (4), 596-606.
(70) AC, W.; RW, J. New and Emerging HDAC Inhibitors for Cancer Treatment. J Clin Invest 2014, 124 (1), 30-39.
(71) von Knethen, A.; Brune, B. Histone Deacetylation Inhibitors as Therapy Concept in Sepsis. International Journal of Molecular Sciences. MDPI AG Jan. 2, 2019.
(72) Kim, H. J.; Bae, S. C. Histone Deacetylase Inhibitors: Molecular Mechanisms of Action and Clinical Trials as Anti-Cancer Drugs. American Journal of Translational Research. e-Century Publishing Corporation 2011, pp 166-179.
(73) Nebbioso, A.; Clarke, N.; Voltz, E.; Germain, E.; Ambrosino, C.; Bontempo, P.; Alvarez, R.; Schiavone, E. M.; Ferrara, F.; Bresciani, F.; Weisz, A.; de Lera, A. R.; Gronemeyer, H.; Altucci, L. Tumor-Selective Action of HDAC Inhibitors Involves TRAIL Induction in Acute Myeloid Leukemia Cells. NATURE MEDICINE 2005, 11 (1), 77.
(74) Shao, Y.; Gao, Z.; Marks, P. A.; Jiang, X. Apoptotic and Autophagic Cell Death Induced by Histone Deacetylase Inhibitors. Proc Natl Acad Sci USA 2004, 101 (52), 18030-18035.
(75) Shah, R. R. Safety and Tolerability of Histone Deacetylase (HDAC) Inhibitors in Oncology. Drug Safety 2019, 42, 235-245.
(76) Kankaanranta, H.; Janka-Junttila, M.; Ilmarinen-Salo, P.; Ito, K.; Jalonen, U.; Ito, M.; Adcock, I. M.; Moilanen, E.; Zhang, X. Histone Deacetylase Inhibitors Induce Apoptosis in Human Eosinophils and Neutrophils. Journal of Inflammation 2010, 7 (1), 1-15.
(77) West, A. C.; Smyth, M. J.; Johnstone, R. W. The Anticancer Effects of HDAC Inhibitors Require the Immune System. OncoImmunology 2014, 3 (1).
(78) Leoni, F.; Zaliani, A.; Bertolini, G.; Porro, G.; Pagani, P.; Pozzi, P.; Dona, G.; Fossati, G.; Sozzani, S.; Azam, T.; Bufler, P.; Fantuzzi, G.; Goncharov, I.; Kim, S. H.; Pomerantz, B. J.; Reznikov, L. L.; Siegmund, B.; Dinarello, C. A.; Mascagni, P. The Antitumor Histone Deacetylase Inhibitor Suberoylanilide Hydroxamic Acid Exhibits Antiinflammatory Properties via Suppression of Cytokines. Proc Natl Acad Sci USA 2002, 99 (5), 2995-3000.
(79) Li, Y.; Liu, B.; Zhao, H.; Sailhamer, E. A.; Fukudome, E. Y.; Zhang, X.; Kheirbek, T.; Finkelstein, R. A.; Velmahos, G. C.; Demoya, M.; Hales, C. A.; Alam, H. B. Protective Effect of Suberoylanilide Hydroxamic Acid against Lps-Induced Septic Shock in Rodents. Shock 2009, 32 (5), 517-523.
(80) Zhao, Y.; Zhou, P.; Liu, B.; Bambakidis, T.; Mazitschek, R.; Alam, H. B.; Li, Y. Protective Effect of Suberoylanilide Hydroxamic Acid against Lipopolysaccharide-Induced Liver Damage in Rodents. Journal of Surgical Research 2015, 194 (2), 544-550.
(81) Li, Y.; Liu, B.; Fukudome, E. Y.; Kochanek, A. R.; Finkelstein, R. A.; Chong, W.; Jin, G.; Lu, J.; Demoya, M. A.; Velmahos, G. C.; Alam, H. B. Surviving Lethal Septic Shock without Fluid Resuscitation in a Rodent Model. Surgery 2010, 148 (2), 246-254.
(82) Chong, W.; Li, Y.; Liu, B.; Zhao, T.; Fukudome, E. Y.; Liu, Z.; Smith, W. M.; Velmahos, G. C.; Demoya, M. A.; Alam, H. B. Histone Deacetylase Inhibitor Suberoylanilide Hydroxamic Acid Attenuates Toll-like Receptor 4 Signaling in Lipopolysaccharide-Stimulated Mouse Macrophages. Journal of Surgical Research 2012, 178 (2), 851-859.
(83) Li, Y.; Zhao, T.; Liu, B.; Halaweish, I.; Mazitschek, R.; Duan, X.; Alam, H. B. Inhibition of Histone Deacetylase 6 Improves Long-Term Survival in a Lethal Septic Model. J Trauma Acute Care Surg 2015, 78 (2), 378.
(84) Tang X. PLGA-PEG Nanoparticles Coated with Anti-CD45RO and Loaded with HDAC Plus Protease Inhibitors Activate Latent HIV and Inhibit Viral Spread_Enhanced Reader. Nanoscale Research Letters 2015.
(85) Patra, J. K.; Das, G.; Fraceto, L. F.; Campos, E. V. R.; Rodriguez-Torres, M. D. P.; Acosta-Torres, L. S.; Diaz-Torres, L. A.; Grillo, R.; Swamy, M. K.; Sharma, S.; Habtemariam, S.; Shin, H. S. Nano Based Drug Delivery Systems: Recent Developments and Future Prospects 10 Technology 1007 Nanotechnology 03 Chemical Sciences 0306 Physical Chemistry (Incl. Structural) 03 Chemical Sciences 0303 Macromolecular and Materials Chemistry 11 Medical and Health Sciences 1115 Pharmacology and Pharmaceutical Sciences 09 Engineering 0903 Biomedical Engineering Prof Ueli Aebi, Prof Peter Gehr. Journal of Nanobiotechnology. BioMed Central Ltd. Sep. 19, 2018.
(86) Casey, L. M.; Kakade, S.; Decker, J. T.; Rose, J. A.; Deans, K.; Shea, L. D.; Pearson, R. M. Cargo-Less Nanoparticles Program Innate Immune Cell Responses to Toll-like Receptor Activation. Biomaterials 2019, 218.
(87) Zhao, J.; Stenzel, M. H. Entry of Nanoparticles into Cells: The Importance of Nanoparticle Properties. Polymer Chemistry. Royal Society of Chemistry Jan. 21, 2018, pp 259-272. Sood, B. G.; Madan, A.; Saha, S.; Schendel, D.; Thorsen, P.; Skogstrand, K.; Hougaard, D.; Shankaran, S.; Carlo, W. Perinatal Systemic Inflammatory Response Syndrome and Retinopathy of Prematurity. Pediatric Research 2010, 67 (4), 394-400.
(88) Chousterman, B. G.; Swirski, F. K.; Weber, G. F. Cytokine Storm and Sepsis Disease Pathogenesis. Seminars in Immunopathology 2017, 39, 517-528.
(89) Sankar, R.; Ravikumar, V. Biocompatibility and Biodistribution of Suberoylanilide Hydroxamic Acid Loaded Poly (DL-Lactide-Co-Glycolide) Nanoparticles for Targeted Drug Delivery in Cancer. Biomedicine & Pharmacotherapy 2014, 68 (7), 865-871.
(90) Marshall, J. C. Why Have Clinical Trials in Sepsis Failed? Trends in Molecular Medicine 2014, 20 (4), 195-203.
(91) VM, R.; BT, T.; PS, B.; JF, D.; IS, D.; S, F.; B, G.; JC, M.; A, R.; A, A.; D, P.; J, T.; HR, A.-K.; V, T.; J, J.; WL, M.; B, V.; MD, W. Drotrecogin Alfa (Activated) in Adults with Septic Shock. N Engl J Med 2012, 366 (22), 2055-2064.
(92) Remick, D. G. Cytokine Therapeutics for the Treatment of Sepsis: Why Has Nothing Worked?; 2003; Vol. 9, pp 75-82.
(93) MP, F. Animal Models of Sepsis. Virulence 2014, 5 (1), 143-153.
(94) MJ, D.; PA, W. Sepsis-Induced Immune Dysfunction: Can Immune Therapies Reduce Mortality? J Clin Invest 2016, 126 (1), 23-31.
(95) Leligdowicz, A.; Matthay, M. A. Heterogeneity in Sepsis: New Biological Evidence with Clinical Applications. Critical Care 2019 23:1 2019, 23 (1), 1-8.
(96) Konsoula, Z.; Cao, H.; Velena, A.; Jung, M. Pharmacokinetics-Pharmacodynamics and Antitumor Activity of Mercaptoacetamide-Based Histone Deacetylase Inhibitors. Mol. Cancer. Ther. 2009, 8 (10).
(97) Makadia, H. K.; Siegel, S. J. Poly Lactic-Co-Glycolic Acid (PLGA) as Biodegradable Controlled Drug Delivery Carrier. Polymers (Basel) 2011, 3 (3), 1377.
(98) Tran, T.; Tran, P. Nanoconjugation and Encapsulation Strategies for Improving Drug Delivery and Therapeutic Efficacy of Poorly Water-Soluble Drugs. Pharmaceutics 2019, 11 (325).
(99) K, D.; K, R.; K, D.; B, T.; K, E.; C, D.; C, H.; M, K.; LA, K.-S.; PJ, O.; R, A.; E, G.; MP, K. Lactic Acid and Acidification Inhibit TNF Secretion and Glycolysis of Human Monocytes. J Immunol 2010, 184 (3), 1200-1209.
(100) Li, Y.; Liu, B.; Gu, X.; Kochanek, A. R.; Fukudome, E. Y.; Liu, Z.; Zhao, T.; Chong, W.; Zhao, Y.; Zhang, D.; Libermann, T. A.; Alam, H. B. Creating a “pro-Survival” Phenotype through Epigenetic Modulation. Surgery (United States) 2012, 152 (3), 455-464.
(101) Binnie, A.; Tsang, J. L. Y.; Hu, P.; Carrasqueiro, G.; Castelo-Branco, P.; dos Santos, C. C. Epigenetics of Sepsis. Critical Care Medicine. Lippincott Williams and Wilkins 2020, pp 745-756.
(102) Roger, T.; Me Lugrin, J.; Roy, D. le; Ve Goy, G.; Mombelli, M.; Koessler, T.; Ding, X. C.; Chanson, A.-L.; Reymond, M. K.; Miconnet, I.; Schrenzel, J.; Frangois, P.; Calandra, T. Histone Deacetylase Inhibitors Impair Innate Immune Responses to Toll-like Receptor Agonists and to Infection. Blood 2011, 117 (4), 1205-1217.
(103) Bode, K.; Schroder, K.; Hume, D. A.; Ravasi, T.; Heeg, K.; Sweet, M. J.; Dalpke, A. H. Histone Deacetylase Inhibitors Decrease Toll-like Receptor-Mediated Activation of Proinflammatory Gene Expression by Impairing Transcription Factor Recruitment. Immunology 2007, 122 (4), 596-606.
(104) Subramanian, S.; Bates, S. E.; Wright, J. J.; Espinoza-Delgado, I.; Piekarz, R. L. Clinical Toxicities of Histone Deacetylase Inhibitors. Pharmaceuticals 2010, 3 (9), 2751-2767.
(105) Gupta, P.; Reid, R. C.; Iyer, A.; Sweet, M. J.; Fairlie, D. P. Towards Isozyme-Selective HDAC Inhibitors For Interrogating Disease; 2012; Vol. 12.
(106) DP, M.; JW, Y.; CT, H.; WK, S.; J, C.; RM, P.; SD, M.; LD, S. An Antigen-Encapsulating Nanoparticle Platform for T H 1/17 Immune Tolerance Therapy. Nanomedicine 2017, 13 (1), 191-200.
(107) Weischenfeldt, J.; Porse, B. Bone Marrow-Derived Macrophages (BMM): Isolation and Applications. Cold Spring Harbor Protocols 2008, 3 (12), pdb.prot5080.
(108) Feldman, A. T.; Wolfe, D. Tissue Processing and Hematoxylin and Eosin Staining. Methods in Molecular Biology 2014, 1180, 31-43.
(109) Rieppo, L.; Janssen, L.; Rahunen, K.; Lehenkari, P.; Finnilä, M. A. J.; Saarakkala, S. Histochemical Quantification of Collagen Content in Articular Cartilage. PLOS One 2019, 14 (11).

1. A compound of Formula (I) 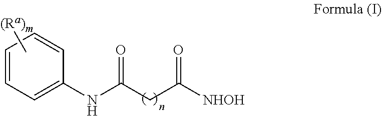
or a pharmaceutically acceptable salt thereof, wherein in Formula (I):
m is 0 or 1;
n is an integer selected from 1, 2, 3, 4, 5, 6, 7, and 8; and
R^{a}, when present, comprises one or more of —OH, C₁-C₃ alkyl, C₁-C₃ alkoxy, —C(O)H, —C(O)OH, and —C(O)X; and
X is halogen selected from F, Br, Cl, and I.

2. The compound of claim 1, wherein n is 6.

3. The compound of claim 1, wherein suberoylanilide hydroxamic acid (SAHA): 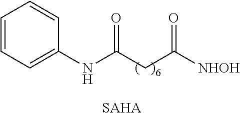
is excluded from Formula (I).

4. The compound of claim 1, wherein m is 1 and n is 6.

5. The compound of claim 4, wherein the compound of Formula (I) is N¹-hydroxy-N⁸-(4-(hydroxymethyl)phenyl)octanediamide (SAHA-OH): 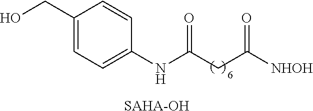
or a pharmaceutically acceptable salt thereof.

6. A conjugate of Formula (10), comprising a polymer moiety (POLY) covalently linked to a drug moiety comprising the compound of claim 1: 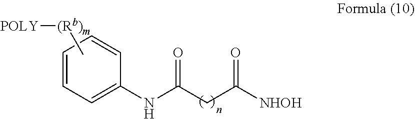
or a pharmaceutically acceptable salt thereof, wherein in Formula (10):
m is 0 or 1;
n is an integer selected from 1, 2, 3, 4, 5, 6, 7, and 8; and
R^{b}, when present, comprises one or more of —O—, —C₁-C₃ alkyl-, —C₁-C₃ alkoxy-, —C(O)—, and —C(O)O—.

7. The conjugate of claim 6, wherein m is 0 and n is 6.

8. The conjugate of claim 6, wherein the conjugate of Formula (10) is 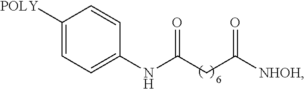
or a pharmaceutically acceptable salt thereof.

9. The conjugate of claim 6, wherein m is 1 and n is 6.

10. The conjugate of claim 6, wherein the conjugate of Formula (10) is 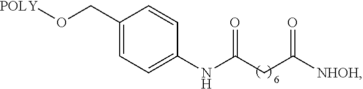
or a pharmaceutically acceptable salt thereof.

11. The conjugate of claim 6, wherein the conjugate of Formula (10) is 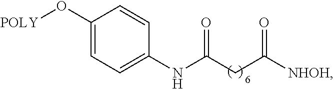
or a pharmaceutically acceptable salt thereof.

12. The conjugate of claim 6, wherein POLY is a biodegradable polymer.

13. The conjugate of claim 6, wherein POLY is selected from polyglycolic acid (PGA), poly(DL-lactide) (PLA), and poly(D,L-lactide-co-glycolide) (PLGA).

14. The conjugate of claim 13, wherein POLY is PLGA.

15. The conjugate of claim 14, wherein PLGA comprises lactic acid monomer subunits and glycolic acid monomer subunits in a ratio from about 25:75 to about 75:25 lactic acid monomer subunits:glycolic acid monomer subunits.

16. The conjugate of claim 14, wherein PLGA comprises lactic acid monomer subunits and glycolic acid monomer subunits in a ratio of from about 25:75 to about 50:50 lactic acid monomer subunits:glycolic acid monomer subunits.

17. The conjugate of claim 14, wherein PLGA comprises lactic acid monomer subunits and glycolic acid monomer subunits in a ratio of from about 50:50 to about 75:25 lactic acid monomer subunits:glycolic acid monomer subunits.

18. The conjugate of claim 14, wherein PLGA comprises lactic acid monomer subunits and glycolic acid monomer subunits in a ratio of about 25:75, about 50:50, or about 75:25 lactic acid monomer subunits:glycolic acid monomer subunits.

19. The conjugate of claim 13, wherein the PLGA comprises an acid terminus which is covalently linked to the drug moiety to form the conjugate of Formula (10).

20. The conjugate of claim 14, wherein POLY is a random copolymer, or has a formula 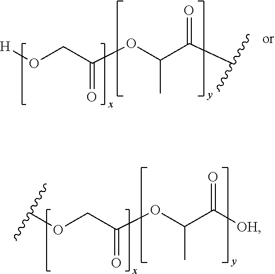
wherein each x and y is independently an integer from 1 to 10,000.

21. The conjugate of claim 6, wherein POLY has a number average molecular weight from about 3 kD to about 50 kD.

22. A nanoparticle comprising a conjugate of claim 6.

23. The nanoparticle of claim 22, further comprising poly(DL-lactide) (PLA).

24. The nanoparticle of claim 23, wherein the PLA is acid-terminated.

25. The nanoparticle of claim 23, wherein the number average molecular weight of PLA is from about 5 kD to about 50 kD.

26. The nanoparticle of claim 22, further comprising a surfactant.

27.-50. (canceled)


SEQUENCE LISTING (2 sequences)

SEQ ID NO: 1 - protein, 7 aa, synthetic construct
  MOD_RES 4: note = Lysine is modified to contain an acetyl group
RHKKAMC

SEQ ID NO: 2 - protein, 7 aa, synthetic construct
  MOD_RES 3: note = Lysine is modified to contain an acetyl group.
  MOD_RES 4: note = Lysine is modified to contain an acetyl group.
RHKKAMC